(19) United States
(12) Patent Application Publication
(10) Pub. No.: US 2007/0191406 A1
(43) Pub. Date: Aug. 16, 2007

(54) Heterocyclic antiviral compounds

(75) Inventors: Rcmy Lemoine, San Francisco, CA (US); Chris Richard Melville, Palo Alto, CA (US); Fernando Padilla, Fremont, CA (US); David Mark Rotstein, Sunnyvale, CA (US); Jutta Wanner, Mountain View, CA (US)

Correspondence Address: ROCHE PALO ALTO LLC;PATENT LAW DEPT. M/S A2-250, 3431 HILLVIEW AVENUE, PALO ALTO, CA, 94304, US

(73) Assignee: Roche Palo Alto LLC

(21) Appl. No.: 11/706,728

(22) Filed: Feb. 15, 2007

Related U.S. Application Data
(60) Provisional application No. 60/773,984, filed on Feb. 15, 2006.

(51) Int. Cl. A61K 31/506; A61K 31/4439; C07D 487/02

(52) U.S. Cl. 514/275; 514/337; 544/331; 546/276.4

(57) ABSTRACT

Chemokine receptor antagonists, in particular, 3,7-diazabicyclo[3.3.0]octane compounds according to formula (I) wherein R¹-R³ R^{6c} and X¹ are as defined herein are antagonists of chemokine CCR5 receptors which are useful for treating or preventing an human immunodeficiency virus (HIV) infection, or treating AIDS or ARC. The invention further provides methods for treating diseases that are alleviated with CCR5 antagonists. The invention includes pharmaceutical compositions and methods of using the compounds for the treatment of these diseases. The invention further includes processes for the preparation of compounds according to formula I.

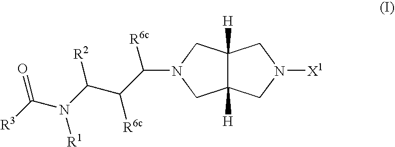

### CROSS REFERENCE TO PRIOR APPLICATIONS

This application claims the benefit of priority to U.S. Ser. No. 60/773,984 filed Feb. 15, 2005 the contents of which are hereby incorporated in their entirety by reference.

### FIELD OF THE INVENTION

This invention relates to octahydro-pyrrolo[3,4-c]pyrrole derivatives useful in the treatment of a variety of disorders, including those in which the modulation of CCR5 receptors is desirable. More particularly, the present invention relates to 3-(hexahydro-pyrrolo[3,4-c]pyrrol-2-yl)-1-phenyl-propylamine and [3-(hexahydro-pyrrolo[3,4-c]pyrrol-2-yl)-propyl]-phenyl-amine compounds and related derivatives, to compositions containing, to uses of such derivatives and to processes for preparing said compounds. Disorders that may be treated or prevented by the present derivatives include HIV and HIV-mediated retroviral infections (and the resulting acquired immune deficiency syndrome, AIDS), diseases of the immune system and inflammatory diseases.

### BACKGROUND OF THE INVENTION

A-M. Vandamme et al. (Antiviral Chemistry & Chemotherapy, 1998 9:187-203) disclose current HAART clinical treatments of HIV-1 infections in man including at least triple drug combinations. Highly active anti-retroviral therapy (HAART) has traditionally consisted of combination therapy with nucleoside reverse transcriptase inhibitors (NRTI), non-nucleoside reverse transcriptase inhibitors (NNRTI) and protease inhibitors (PI). These compounds inhibit biochemical processes required for viral replication. In compliant drug-naive patients, HAART is effective in reducing mortality and progression of HIV-1 to AIDS. While HAART has dramatically altered the prognosis for HIV infected persons, there remain many drawbacks to the current therapy including highly complex dosing regimes and side effects which can be very severe (A. Carr and D. A. Cooper, Lancet 2000 356(9239):1423-1430). Moreover, these multidrug therapies do not eliminate HIV-1 and long-term treatment usually results in multidrug resistance, thus limiting their utility in long term therapy. Development of new drug therapies to provide better HIV-1 treatment remains a priority.

The chemokines are a large family of pro-inflammatory peptides that exert their pharmacological effect through G-protein-coupled receptors. The CCR5 receptor is one member of this family. The chemokines are leukocyte chemotactic proteins capable of attracting leukocytes to various tissues, which is an essential response to inflammation and infection. The name “chemokine”, is a contraction of “chemotactic cytokines”. Human chemokines include approximately 50 structurally homologous small proteins comprising 50-120 amino acids. (M. Baggiolini et al., Ann. Rev. Immunol. 1997 15:675-705)

The CCR5 receptor is a chemokine receptor. The chemokines are a subset of the cytokine family of soluble immune mediators. Chemokine receptors are seven membrane-spanning receptors that signal through heterotrimeric G protein when bound to an agonist. Human CCR5 is composed of 352 amino acids with an intra-cellular C-terminus containing structural motifs for G-protein association and ligand-dependent signaling (M. Oppermann Cellular Signaling 2004 16:1201-1210). The extra-cellular N-terminal domain contributes to high-affinity chemokine binding and interactions with the gp120 HIV protein (T. Dragic J. Gen. Virol. 2001 82:1807-1814; C. Blanpain et al. J. Biol. Chem. 1999 274:34719-34727). The binding site for the natural agonist RANTES (Regulated upon Activation and is Normal T-cell Expressed and Secreted) has been shown to be on the N-terminal domain and HIV gp120 has been suggested to interact initially with the N-terminal domain and also with the ECL2. (B. Lee, et al. J. Biol. Chem. 1999 274:9617-26)]

Modulators of the CCR5 receptor may be useful in the treatment of various inflammatory diseases and conditions, and in the treatment of infection by HIV-1 and genetically related retroviruses. As leukocyte chemotactic factors, chemokines play an indispensable role in the attraction of leukocytes to various tissues of the body, a process which is essential for both inflammation and the body's response to infection. Because chemokines and their receptors are central to the pathophysiology of inflammatory, autoimmune and infectious diseases, agents which are active in modulating, preferably antagonizing, the activity of chemokines and their receptors, are useful in the therapeutic treatment of these diseases. The CCR5 receptor is of particular importance in the context of treating inflammatory and infectious diseases. The natural ligands for the CCR5 are the macrophage inflammatory proteins (MIP) designated MIP-1a and MIP-1b and RANTES.

HIV-1 infects cells of the monocyte-macrophage lineage and helper T-cell lymphocytes by exploiting a high affinity interaction of the viral enveloped glycoprotein (Env) with the CD4 antigen. The CD4 antigen, however appeared to be a necessary, but not sufficient requirement for cell entry and at least one other surface protein was required to infect the cells (E. A. Berger et al., Ann. Rev. Immunol. 1999 17:657-700). Two chemokine receptors, either the CCR5 or the CXCR4 receptor were subsequently found to be co-receptors which are required, along with CD4, for infection of cells by the human immunodeficiency virus (HIV). The central role of CCR5 in the pathogenesis of HIV was inferred by epidemiological identification of powerful disease modifying effects of the naturally occurring null allele CCR5 Δ32. The Δ32 mutation has a 32-base pair deletion in the CCR5 gene resulting in a truncated protein designated Δ32. Relative to the general population, Δ32/Δ32 homozygotes are significantly common in exposed/uninfected individuals suggesting the role of CCR5 in HIV cell entry (R. Liu et al., Cell 1996 86(3):367-377; M. Samson et al., Nature 1996 382(6593):722-725).

The HIV-1 envelope protein is comprised of two subunits: gp120, the surface subunit and gp41, the transmembrane subunit. The two subunits are non-covalently associated and form homotrimers which compose the HIV envelope. Each gp41 subunit contains two helical heptad repeat regions, HR1 and HR2 and a hydrophobic fusion region on the C-terminus.

The CD4 binding site on the gp120 of HIV appears to interact with the CD4 molecule on the cell surface inducing a conformation change in gp120 which creates or exposes a cryptic CCR5 (or CXCR4) binding site, and undergoes conformational changes which permits binding of gp120 to the CCR5 and/or CXCR4 cell-surface receptor. The bivalent interaction brings the virus membrane into close proximity with the target cell membrane and the hydrophobic fusion region can insert into the target cell membrane. A conformation change in gp41 creates a contact between the outer leaflet of the target cell membrane and the viral membrane which produces a fusion pore whereby viral core containing genomic RNA enters the cytoplasm

Viral fusion and cell entry is a complex multi-step process and each step affords the potential for therapeutic intervention. These steps include (i) CD40-gp120 interactions, (ii) CCR5 and/or CXCR4 interactions and (iii) gp41 mediated membrane fusion. Conformational changes induced by these steps expose additional targets for chemotherapeutic intervention. Each of these steps affords an opportunity for therapeutic intervention in preventing or slowing HIV infection. Small molecules (Q. Guo et al. J. Virol. 2003 77:10528-63) and antibodies (D. R. Kuritzkes et al. 10^{th} Conference on Retroviruses and Opportunistic Infections, Feb. 10-14, 2003, Boston, Mass. Abstract 13; K. A. Nagashima et al. J. Infect. Dis. 2001 183:1121-25) designed to prevent the gp120/CD4 interaction have been disclosed. Small molecule antagonists of, and antibodies to, CCR5 are discussed below. A small molecular weight antagonist of CXCR4 has been explored (J. Blanco et al. Antimicrob. Agents Chemother. 2000 46:1336-39). Enfuvirtide (T20, ENF or FUZEON®) is a 36 amino acid peptide corresponding to residues 643-678 in the HR2 domain of gp41. Enfuvirtide binds to the trimeric coiled-coil by the HRI domains and acts in a dominant negative manner to block the endogenous six helix bundle formation thus inhibiting viral fusion. (J. M. Kilby et al., New Eng. J. Med. 1998 4(11):1302-1307). Enfuvirtide has been approved for clinical use.

In addition to the potential for CCR5 modulators in the management of HIV infections, the CCR5 receptor is an important regulator of immune function and compounds of the present invention may prove valuable in the treatment of disorders of the immune system. Treatment of solid organ transplant rejection, graft v. host disease, arthritis, rheumatoid arthritis, inflammatory bowel disease, atopic dermatitis, psoriasis, asthma, allergies or multiple sclerosis by administering to a human in need of such treatment an effective amount of a CCR5 antagonist compound of the present invention is also possible. (M. A. Cascieri and M. S. Springer, Curr. Opin. Chem. Biol. 2000 4:420-427; A. Proudfoot et al., Immunol. Rev. 2000 177:246-256; P. Houshmand and A. Zlotnik, Curr. Opin. Chem. Biol. 2003 7:457-460)

Related octahydro-pyrrolo[3,4-c]pyrrole compounds which antagonists of the CCR5 receptor have been disclosed by E. K. Lee et al. in WO 2005121145 entitled Preparation of heterocyclic antiviral compounds, particularly (3-hexahydropyrrolo[3,4-c]pyrrol-2-yl)-1-phenylpropylamine and [3-(hexahydropyrrolo[3,4-c]pyrrol-2-yl)propyl]phenylamine derivatives as antagonists of chemokine CCR5 receptor, useful for treating HIV and genetically related retroviral infections, published Dec. 22, 2005 which is hereby incorporated by reference in its entirety

### SUMMARY OF THE INVENTION

The present invention relates to a compounds according to formula I which are CCR5 receptor antagonists, methods for treating diseases alleviated by administration of a compound according to formula I and pharmaceutical compositions for treating diseases containing a compound according to formula I admixed with at least one carrier, diluent or excipient, 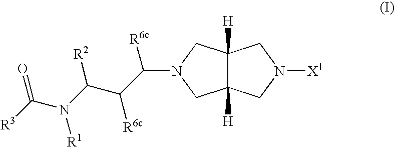
one of R¹ and R² is phenyl optionally substituted with one to four substituents selected independently in each incidence from the group consisting of halogen, C₁₋₆ alkyl, cyano and C₁₋₆ alkoxy; and, the other of R¹ and R² is hydrogen;
R⁵ is hydroxy, NR^{6a}R^{6b}, C₁₋₆ alkoxy or benzyloxy;
R⁶ is hydrogen, C₁₋₆ alkyl, C₁₋₃ haloalkyl, C₁₋₆ hydroxyalkyl or oxo-C₁₋₆ alkyl;
R^{6a}, R^{6b}, R^{6c} and R^{6d} are independently hydrogen or C₁₋₃ alkyl with the proviso that at least one of R^{6c} is hydrogen;
X¹ is selected from the group consisting of (i)-(xiii) and (xiv):
wherein
   X² is N or CH;
   A¹ is C₁₋₆ straight or branched alkylene optionally substituted by a phenyl ring or phenylene;
   m is zero to two; 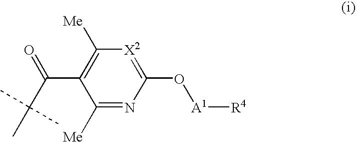
   wherein R⁴ is C(═O)R⁵ or hydrogen; 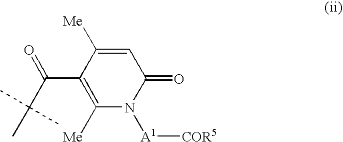
   with the proviso that A¹ is other than phenylene; 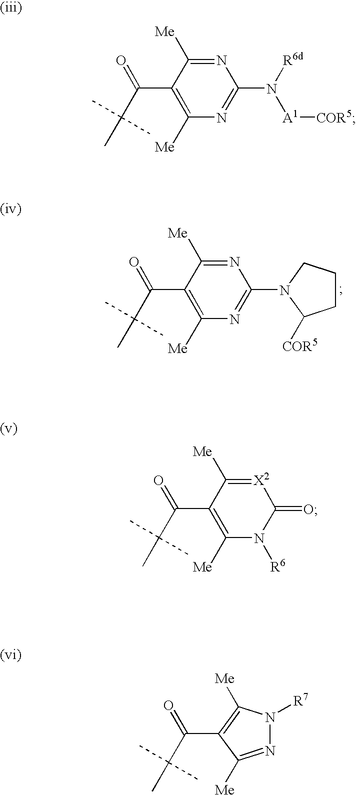
   wherein:
      R⁷ is C₃₋₇ cycloalkyl, (CH₂)ₙCOR⁵, heteroaryl selected from the group consisting of pyridine, pyrimidine, pyrazine and pyridazine said heteroaryl optionally substituted with C₁₋₃ alkyl or C₁₋₃ haloalkyl;
      n is 1 to 3; 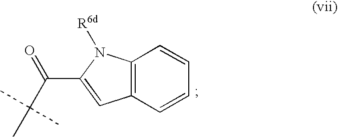 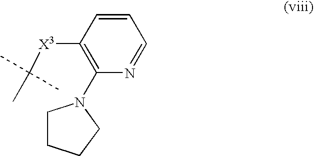
      wherein X³ is —S(O)₂— or —C(O)—; 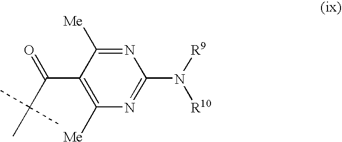
      wherein
      R⁹ and R¹⁰ are (A) together a group (CH₂)₂X⁴(CH₂)₂, (CH₂)₂CH(R¹²)CH₂, or (CH₂)₂SO₂; or, (B) independently R¹⁰ is hydrogen or C₁₋₃ alkyl and R⁹ is —SO₂C₁₋₆ alkyl, C₁₋₆ hydroxyalkyl, xA, xB or xC; 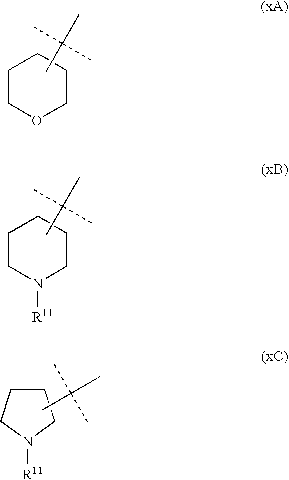
      X⁴ is O, S(O)ₘ, NR¹¹ or CH(NHSO₂C₁₋₆ alkyl);
      R¹¹ is R^{6d}, —C(O)C₁₋₆ alkyl, S(O)₂C₁₋₆ alkyl;
      R¹² is hydrogen, hydroxyl or C₁₋₁₀ acyloxy;
      m is zero to two; and, 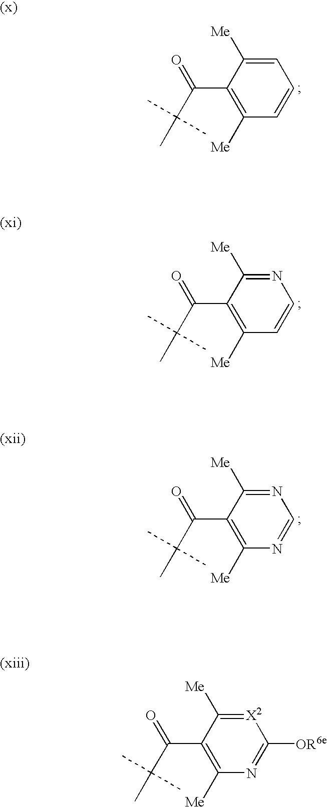
      wherein R^{6e} is C₁₋₆ hydroxy alkyl or oxo-C₁₋₆ alkyl; and, 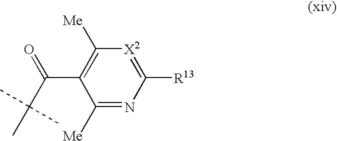
      wherein R¹³ is C₃₋₅ cycloalkyl or C₁₋₃ alkynyl;
R³ is selected from the group consisting of (i), (ii), (iii), (iv) and (v) wherein:
   (i) C₃₋₇ cycloalkyl substituted one or more substituents selected from the group consisting of C₁₋₆ alkoxy, CO₂R^{6d}, CONR^{6a}R^{6b}, fluorine, —NR^{6d}CO C₁₋₃alkyl, —NR^{6d}SO₂ C₁₋₃ alkyl, and C₁₋₁₀ acyloxy or two hydrogens on the same carbon together are replaced by oxygen (oxo) provided that R³ is not 4-oxo-cyclohexyl or 3-oxo-cyclobutyl and when the cycloalkyl is substituted with fluorine, R² is meta-cyano-phenyl; 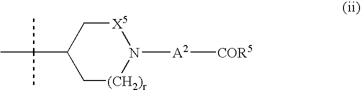
   wherein:
      A² is C₁₋₆ straight or branched alkylene wherein one carbon atom can optionally be replaced by —O—, —S(O)ₘ—, or NR⁵ providing the carbon replaced is not bonded to the heterocyclic nitrogen or the terminal carboxy moiety or A² is absent and R⁵ is tert-butyl;
      X⁵ is C(═O) or CH₂;
      r is zero or one; 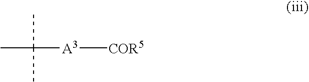
   wherein A³ is C₁₋₆ alkylene said alkylene optionally substituted with C₅₋₇ cycloalkyl or A³-COR⁵ together represent NH(CH₂)ₙCOR⁵; n is one to three; 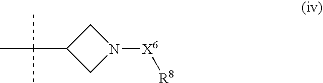
   wherein:
      X⁶ is C(O)R⁸ or S(O)₂C₁₋₆ alkyl;
      R⁸ is C₁₋₆ alkyl, C₁₋₆ haloalkyl, C₃₋₇ cycloalkyl, C₃₋₇ cycloalkyl-C₁₋₃ alkyl; C₁₋₆ alkoxy or C₁₋₆ alkylamino; with the proviso that when R³ is (iv), X¹ is not (x), (xi) or (xii);
   (v) phenylamine optionally substituted with —SO₂NH₂; and,
      pharmaceutically acceptable salts, hydrates and solvates.

### DETAILED DESCRIPTION OF THE INVENTION

In one embodiment of the present invention there is provided a compound according to formula I wherein R¹, R², R³, R⁴, R⁵, R⁶, R^{6a}, R^{6b}, R^{6c}, R^{6d}, R^{6e}, R⁷, R⁸, R⁹, R¹⁰, R¹¹, R¹², R¹³, X¹, X², X³, X⁴, X⁵, X⁶, A¹, A², A³, m, n and r are as described herein above. The phrase “as defined herein above” refers to the broadest definition for each group as provided in the Summary of the Invention or the broadest claim. In all other embodiments provided below, substituents which can be present in each embodiment and which are not explicitly defined retain the broadest definition provided in the Summary of the Invention.

In another embodiment of the present invention there is provided a compound according to formula I wherein R⁶ is hydrogen or C₁₋₆ alkyl; X¹ is (i)-(xi) or (xii); R⁹ and R¹⁰ are (A) together a group (CH₂)₂X⁴(CH₂)₂ or (B) R¹⁰ is hydrogen or C₁₋₃ alkyl and R⁹ is —SO₂C₁₋₆ alkyl, xA or xB; R³ is selected from the group consisting of (i), (ii), (iii) and (iv) wherein (i) C₃₋₇ cycloalkyl substituted with C₁₋₆ alkoxy, CO₂R^{6d}, CONR^{6a}R^{6b} or two hydrogens on the same carbon together are replaced by oxygen (oxo) with the proviso that R³ is not 4-oxo-cyclohexyl or 3-oxo-cyclobutyl.

In another embodiment of the present invention there is provided a compound according to formula I wherein X¹ is (x), (xi) or (xii).

In another embodiment of the present invention there is provided a compound according to formula I wherein X¹ is (i)-(ix) and R³ is (i)-(iv).

In a further embodiment of the present invention there is provided a compound according to formula I wherein X¹ is (ix), (xiii) or (xiv) and R⁹ and R¹⁰ are (A) together a group (CH₂)₂SO₂ or (B) R¹⁰ is hydrogen or C₁₋₃ alkyl and R⁹ is C₁₋₆ hydroxyalkyl, xC.

In yet another embodiment of the present invention there is provided a compound according to formula I wherein X¹ is (x), (xi) or (xii) and R³ is C₃₋₇ cycloalkyl substituted with C₁₋₆ alkoxy, CO₂R⁶, CONR^{6a}R^{6b} or two hydrogens on the same carbon together are replaced by oxygen (oxo) wherein R^{6a} and R^{6b} are independently R⁶ and provided that R³ is not 4-oxo-cyclohexyl or 3-oxo-cyclobutyl.

In another embodiment of the present invention there is provided a compound according to formula I wherein X¹ is (x), (xi) or (xii) and R³ is C₃₋₇ cycloalkyl substituted with CO₂R⁶, 3-oxo-cyclopentyl or 3-oxo-cyclohexyl.

In another embodiment of the present invention there is provided a compound according to formula I wherein X¹ is (x), (xi) or (xii) and R³ is (ii).

In another embodiment of the present invention there is provided a compound according to formula I wherein X¹ is (x), (xi) or (xii) and R³ is (ii) wherein A¹ is C₁₋₆ straight or branched alkylene; X³ is CH₂; and, r is one.

In another embodiment of the present invention there is provided a compound according to formula I wherein X¹ is (i), (ii), (iii), (iv), (v), (vi), (vii), (viii), (ix), (xiii) or (xiv).

In another embodiment of the present invention there is provided a compound according to formula I wherein X¹ is (vi) and R⁷ is heteroaryl selected from the group consisting of pyridine, pyrimidine, pyrazine and pyridazine said heteroaryl optionally substituted with C₁₋₃ alkyl or C₁₋₃ haloalkyl.

In another embodiment of the present invention there is provided a compound according to formula I wherein X¹ is (v) and R⁶ is C₁₋₃ alkyl.

In another embodiment of the present invention there is provided a compound according to formula I wherein X¹ is (i) or (iii); R⁵ is hydroxyl, C₁₋₆ alkoxy, or NR^{6a}R^{6b} and R^{6a} and R^{6b} are hydrogen.

In another embodiment of the present invention there is provided a compound according to formula I which compound is selected from compounds I-1 to I-43 of TABLE 1.

In another embodiment of the present invention there is provided a method for treating or preventing an human immunodeficiency virus (HIV) infection, or treating AIDS or ARC, in a patient in need thereof which comprises administering to the patient a therapeutically effective amount of a compound of formula I wherein R¹, R², R³, R⁴, R⁵, R⁶, R^{6a}, R^{6b}, R^{6c}, R^{6d}, R^{6e}, R⁷, R⁸, R⁹, R¹⁰, R¹¹, R¹², R¹³, X¹, X², X³, X⁴, X⁵, X⁶, A¹, A², A³, m, n and r are as described herein above.

In another embodiment of the present invention there is provided a method for treating or preventing an human immunodeficiency virus (HIV) infection, or treating AIDS or ARC, in a patient in need thereof which comprises co-administering a therapeutically effective amount of at least one compound selected from the group consisting of HIV nucleoside reverse transcriptase inhibitors, HIV non-nucleoside reverse transcriptase inhibitors, HIV protease inhibitors and viral fusion inhibitors in additional to a compound of formula I wherein R¹, R², R³, R⁴, R⁵, R⁶, R^{6a}, R^{6b}, R^{6c}, R^{6d}, R^{6e}, R⁷, R⁸, R⁹, R¹⁰, R¹¹, R¹², R¹³, X¹, X², X³, X⁴, X⁵, X⁶, A¹, A², A³, m, n and r are as described herein above.

In another embodiment of the present invention there is provided a method for treating or preventing an human immunodeficiency virus (HIV) infection, or treating AIDS or ARC, in a patient in need thereof which comprises co-administering a therapeutically effective amount of at least one of efavirenz, nevirapine, delavirdine, zidovudine, didanosin, zalcitabine, stavudine, lamivudine, abacavir, adefovir and dipivoxil, saquinavir, ritonavir, nelfinavir, indinavir, amprenavir, lopinavir or T-20 in additional to a compound of formula I wherein R¹, R², R³, R⁴, R⁵, R⁶, R^{6a}, R^{6b},R^{6c}, R^{6d}, R^{6e}, R⁷, R⁸, R⁹, R¹⁰, R¹¹, R¹², R¹³, X¹, X², X³, X⁴, X⁵, X⁶, A¹, A², A³, m, n and r are as described herein above.

In another embodiment of the present invention there is provided a method for treating a mammal with a disease state that is alleviated by a CCR⁵ receptor antagonist wherein said disease is solid organ transplant rejection, graft v. host disease, arthritis, rheumatoid arthritis, inflammatory bowel disease, atopic dermatitis, psoriasis, asthma, allergies or multiple sclerosis which comprises administering to the mammal in need thereof a therapeutically effective amount of a compound of formula I wherein R¹, R², R³, R⁴, R⁵, R⁶, R^{6a}, R^{6b}, R^{6c}, R^{6d}, R^{6e}, R⁷, R⁸, R⁹, R¹⁰, R¹¹, R¹²,R¹³, X¹, X², X³, X⁴, X⁵, X⁶, A¹, A², A³, m, n and r are as described herein above.

In another embodiment of the present invention there is provided a method for treating a mammal with a disease state that is alleviated by a CCR⁵ receptor antagonist wherein said disease is solid organ transplant rejection, graft v. host disease, arthritis, rheumatoid arthritis, inflammatory bowel disease, atopic dermatitis, psoriasis, asthma, allergies or multiple sclerosis which comprises co-administering to the mammal in need thereof a therapeutically effective amount at least one other immune modulator and a compound of formula I wherein R¹, R², R³, R⁴, R⁵, R⁶, R^{6a}, R^{6b}, R^{6c}, R^{6d}, R^{6e}, R⁷, R⁸, R⁹, R¹⁰, R¹¹, R¹², R¹³, X¹, X², X³, X⁴, X⁵, X⁶, A¹, A², A³, m, n and r are as described herein above.

In another embodiment of the present invention there is provided a method for treating a human with a disease state that is alleviated by a CCR⁵ receptor antagonist wherein said disease is solid organ transplant rejection, graft v. host disease, arthritis, rheumatoid arthritis, inflammatory bowel disease, atopic dermatitis, psoriasis, asthma, allergies or multiple sclerosis which comprises co-administering to the mammal in need thereof a therapeutically effective amount at least one other immune modulator and a compound of formula I wherein R¹, R², R³, R⁴, R⁵, R⁶, R^{6a}, R^{6b}, R^{6c}, R^{6d}, R^{6e}, R⁷, R⁸, R⁹, R¹⁰, R¹¹, R¹², R¹³, X¹, X², X³, X⁴, X⁵, X⁶, A¹, A², A³, m, n and r are as described herein above.

In another embodiment of the present invention there is provided a pharmaceutical composition for treating or preventing an human immunodeficiency virus (HIV) infection, or treating AIDS or ARC comprising a compound according to formula I wherein R¹, R², R³, R⁴, R⁵, R⁶, R^{6a}, R^{6b}, R^{6c}, R^{6d}, R^{6e}, R⁷, R⁸, R⁹, R¹⁰, R¹¹, R¹², R¹³, X¹, X², X³, X⁴, X⁵, X⁶, A¹, A², A³, m, n and r are as described herein above said compound of formula I admixed with at least one pharmaceutical acceptable carrier, diluent or excipient.

In another embodiment of the present invention there is provided a pharmaceutical composition for treating a mammal with a disease state that is alleviated by a CCR⁵ receptor antagonist wherein said disease is solid organ transplant rejection, graft v. host disease, arthritis, rheumatoid arthritis, inflammatory bowel disease, atopic dermatitis, psoriasis, asthma, allergies or multiple sclerosis comprising a compound according to formula I wherein R¹, R², R³, R⁴, R⁵, R⁶, R^{6a}, R^{6b}, R^{6c}, R^{6d}, R^{6e}, R⁷, R⁸, R⁹, R¹⁰, R¹¹, R¹², R¹³, X¹, X², X³, X⁴, X⁵, X⁶, A¹, A², A³, m, n and r are as described herein above said compound of formula I admixed with at least one pharmaceutical acceptable carrier, diluent or excipient.

### DEFINITIONS

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The term “optional” or “optionally” as used herein means that a subsequently described event or circumstance may, but need not, occur, and that the description includes instances where the event or circumstance occurs and instances in which it does not. For example, “optionally substituted” means that the moiety may be hydrogen or a substituent.

It is contemplated that the definitions described herein may be appended to form chemically-relevant combinations, such as “heteroalkylaryl,” “haloalkylheteroaryl,” “arylalkylheterocyclyl,” “alkylcarbonyl,” “alkoxyalkyl,” and the like.

The term “alkyl” as used herein denotes an unbranched or branched chain, saturated, monovalent hydrocarbon residue containing 1 to 6 carbon atoms. The term “lower alkyl” denotes a straight or branched chain hydrocarbon residue containing 1 to 4 carbon atoms. “C₁₋₁₀ alkyl” as used herein refers to an alkyl composed of 1 to 10 carbons. One or more of the carbon atoms may optionally be replaced by oxygen, sulfur, substituted or unsubstituted nitrogen atom(s). Examples of alkyl groups include, but are not limited to, lower alkyl groups include methyl, ethyl, propyl, i-propyl, n-butyl, i-butyl, t-butyl or pentyl, isopentyl, neopentyl, hexyl, heptyl, and octyl.

When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” denotes the radical R′R″—, wherein R′ is a phenyl radical, and R″ is an alkylene radical as defined herein with the understanding that the attachment point of the phenylalkyl moiety will be on the alkylene radical. Examples of arylalkyl radicals include, but are not limited to, benzyl, phenylethyl, 3-phenylpropyl. The terms “arylalkyl” or “aralkyl” are interpreted similarly except R′ is an aryl radical. The terms “(het)arylalkyl” or “(het)aralkyl” are interpreted similarly except R′ is optionally an aryl or a heteroaryl radical. An “alkylaminoalkyl” is an alkyl group having one to two alkylamino substituents. “Hydroxyalkyl” includes 2-hydroxyethyl, 2-hydroxypropyl, 1-(hydroxymethyl)-2-methylpropyl, 2-hydroxybutyl, 2,3-dihydroxybutyl, 2-(hydroxymethyl), 3-hydroxypropyl, and so forth. Accordingly, as used herein, the term “hydroxyalkyl” is used to define a subset of heteroalkyl groups defined below.

The term “alkylene” as used herein denotes a divalent saturated linear hydrocarbon radical of 1 to 6 carbon atoms (e.g., (CH₂)ₙ)or a branched saturated divalent hydrocarbon radical of 2 to 6 carbon atoms (e.g., —CHMe- or —CH₂CH(i-Pr)CH₂—), unless otherwise indicated. The open valences of an alkylene group are not attached to the same atom. Examples of alkylene radicals include, but are not limited to, methylene, ethylene, propylene, 2-methyl-propylene, butylene, 2-ethylbutylene.

The term “haloalkyl” as used herein denotes an unbranched or branched chain alkyl group as defined above wherein 1, 2, 3 or more hydrogen atoms are substituted by a halogen. Examples are 1-fluoromethyl, 1-chloromethyl, 1-bromomethyl, 1-iodomethyl, difluoromethyl, trifluoromethyl, trichloromethyl, tribromomethyl, triiodomethyl, 1-fluoroethyl, 1-chloroethyl, 1-bromoethyl, 1-iodoethyl, 2-fluoroethyl, 2-chloroethyl, 2-bromoethyl, 2-iodoethyl, 2,2-dichloroethyl, 3-bromopropyl or 2,2,2-trifluoroethyl.

The term “cyano” as used herein refers to a carbon linked to a nitrogen by a triple bond, i.e., —C≡N.

The term “acyl” as used herein denotes a group of formula —C(═O)R wherein R is hydrogen or lower alkyl as defined herein. The term or “alkylcarbonyl” as used herein denotes a group of formula C(═O)R wherein R is alkyl as defined herein. The term “arylcarbonyl” as used herein means a group of formula C(═O)R wherein R is an aryl group; the term “benzoyl” as used herein an “arylcarbonyl” group wherein R is phenyl.

The term “acyloxy” as used herein denotes the radical —OC(O)R, wherein R is a lower alkyl radical as defined herein. Examples of acyloxy radicals include, but are not limited to, acetoxy, propionyloxy.

The term “alkoxy” as used herein means an —O-alkyl group, wherein alkyl is as defined above such as methoxy, ethoxy, n-propyloxy, i-propyloxy, n-butyloxy, i-butyloxy, t-butyloxy, pentyloxy, hexyloxy, including their isomers. “Lower alkoxy” as used herein denotes an alkoxy group with a “lower alkyl” group as previously defined. “C₁₋₁₀ alkoxy” as used herein refers to an —O-alkyl wherein alkyl is C₁₋₁₀.

The term “halogen” or “halo” as used herein means fluorine, chlorine, bromine, or iodine.

The term “aryl” as used herein denotes a monovalent aromatic carbocyclic radical containing 5 to 15 carbon atoms consisting of one individual ring, or one or more fused rings in which at least one ring is aromatic in nature, which can optionally be substituted with one or more, preferably one or three substituents independently selected from hydroxy, thio, cyano, alkyl, alkoxy, lower haloalkoxy, alkylthio, halogen, haloalkyl, hydroxyalkyl, nitro, alkoxycarbonyl, amino, alkylamino, dialkylamino, aminoalkyl, alkylaminoalkyl, and dialkylaminoalkyl, alkylsulfonyl, arylsulfinyl, alkylaminosulfonyl, arylaminosulfonyl, alkylsulfonylamino, arylsulfonylamino, carbamoyl, alkylcarbamoyl and dialkylcarbamoyl, arylcarbamoyl, alkylcarbonylamino, arylcarbonylamino, unless otherwise indicated. Alternatively two adjacent atoms of the aryl ring may be substituted with a methylenedioxy or ethylenedioxy group. Thus a bicyclic aryl substituents may be fused to a heterocyclyl or heteroaryl ring; however, the point of attachment of bicyclic aryl substituent is on the carbocyclic aromatic ring. Examples of aryl radicals include, phenyl, naphthyl, indanyl, anthraquinolyl, tetrahydronaphthyl, 3,4-methylenedioxyphenyl, 1,2,3,4-tetrahydroquinolin-7-yl, 1,2,3,4-tetrahydroisoquinoline-7-yl, and the like. The term “phenylene” refers to a divalent phenyl ring which can by o-, m- or p-phenylene.

The term “heteroaryl” or “heteroaromatic” as used herein means a monocyclic or bicyclic radical of 5 to 12 ring atoms having at least one aromatic ring containing four to eight atoms per ring, incorporating one or more N, O, or S heteroatoms, the remaining ring atoms being carbon, with the understanding that the attachment point of the heteroaryl radical will be on a heteroaryl ring. As well known to those skilled in the art, heteroaryl rings have less aromatic character than their all-carbon counter parts. Thus, for the purposes of the invention, a heteroaryl group need only have some degree of aromatic character. Examples of heteroaryl moieties include monocyclic aromatic heterocycles having 5 to 6 ring atoms and 1 to 3 heteroatoms include, but is not limited to, pyridinyl, pyrimidinyl, pyrazinyl, pyrrolyl, pyrazolyl, imidazolyl, oxazolyl, isoxazolyl, thiazolyl, isothiazolyl, triazolinyl, thiadiazolyl and oxadiaxolinyl which can optionally be substituted with one or more, preferably one or two substituents selected from hydroxy, cyano, alkyl, alkoxy, thio, lower haloalkoxy, alkylthio, halo, haloalkyl, alkylsulfinyl, alkylsulfonyl, halogen, amino, alkylamino, dialkylamino, aminoalkyl, alkylaminoalkyl, and dialkylaminoalkyl, nitro, alkoxycarbonyl and carbamoyl, alkylcarbamoyl, dialkylcarbamoyl, arylcarbamoyl, alkylcarbonylamino and arylcarbonylamino. Examples of bicyclic moieties include, but are not limited to, quinolinyl, isoquinolinyl, benzofuryl, benzothiophenyl, benzoxazole, benzisoxazole, benzothiazole and benzisothiazole. Bicyclic moieties can be optionally substituted on either ring; however the point of attachment is on a ring containing a heteroatom.

The term “heterocyclyl” or “heterocycle” as used herein denotes a monovalent saturated cyclic radical, consisting of one or more rings, preferably one to two rings, of three to eight atoms per ring, incorporating one or more ring heteroatoms (chosen from N, O or S(O)0-2), and which can optionally be independently substituted with one or more, preferably one or two substituents selected from hydroxy, oxo, cyano, lower alkyl, lower alkoxy, lower haloalkoxy, alkylthio, halo, haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminocarbonyl, arylaminocarbonyl, alkylcarbonylamino, arylcarbonylamino, unless otherwise indicated. A bicyclic heterocycle can be fused to an aryl or heteroaryl ring; however, the point of attachment is on the heterocyclic ring. Examples of heterocyclic radicals include, but are not limited to, azetidinyl, pyrrolidinyl, hexahydroazepinyl, oxetanyl, tetrahydrofuranyl, tetrahydrothiophenyl, oxazolidinyl, thiazolidinyl, isoxazolidinyl, morpholinyl, piperazinyl, piperidinyl, tetrahydropyranyl, thiomorpholinyl, quinuclidinyl and imidazolinyl.

The term “cycloalkyl” as used herein denotes a saturated carbocyclic ring containing 3 to 8 carbon atoms, i.e. cyclopropyl, cyclobutyl, cyclopentyl, cyclohexyl, cycloheptyl or cyclooctyl. “C₃₋₇ cycloalkyl” as used herein refers to an cycloalkyl composed of 3 to 7 carbons in the carbocyclic ring.

The term “oxetane” refers to a four-membered saturated heterocycle containing one oxygen atom. “Oxetanyl” refers to an oxetane radical.

Compounds of formula I exhibit tautomerism. Tautomeric compounds can exist as two or more interconvertable species. Prototropic tautomers result from the migration of a covalently bonded hydrogen atom between two atoms. Tautomers generally exist in equilibrium and attempts to isolate an individual tautomers usually produce a mixture whose chemical and physical properties are consistent with a mixture of compounds. The position of the equilibrium is dependent on chemical features within the molecule. For example, in many aliphatic aldehydes and ketones, such as acetaldehyde, the keto form predominates while; in phenols, the enol form predominates. Common prototropic tautomers include keto/enol (—C(═O)—CH—⇄—C(—OH)═CH—), amide/imidic acid (—C(═O)—NH—⇄—C(—OH)═N—) and amidine (—C(═NR)—NH—⇄—C(—NHR)═N—) tautomers. The latter two are particularly common in heteroaryl and heterocyclic rings and the present invention encompasses all tautomeric forms of the compounds.

The term “protecting group” as used herein refers to a chemical group that (a) efficiently combines with a reactive group in a molecule; (b) prevents a reactive group from participating in an undesirable chemical reaction; and (c) can be easily removed after protection of the reactive group is no longer required. Protecting groups are used in synthesis to temporarily mask the characteristic chemistry of a functional group because it interferes with another reaction. Reagents and protocols for to introduce and remove protecting groups are well known and have been reviewed in numerous texts (e.g., T. W. Greene and P. G. M. Wuts, Protective Groups in Organic Synthesis, 3^{rd} edition, John Wiley & Sons, New York, 1999, and Harrison and Harrison et al., Compendium of Synthetic Organic Methods, Vols. 1-8 John Wiley and Sons, 1971-1996). One skilled in the chemical arts will appreciate that on occasion protocols must be optimized for a particular molecule and such optimization is well with the ability of one skilled in these arts. Amino-protecting groups used extensively herein include N-urethanes such as the N-benzyloxycarbonyl group (cbz) or tert-butoxycarbonyl (BOC) which is prepared by reaction with di(t-butyl)dicarbonate and benzyl groups. Benzyl groups are removed conveniently by hydrogenolysis and BOC groups are labile under acidic conditions.

It will be appreciated by the skilled artisan that the compounds of formula I may contain one or more chiral centers and therefore exist in two or more stereoisomeric forms. The racemates of these isomers, the individual isomers and mixtures enriched in one enantiomer, as well as diastereomers when there are two chiral centers, and mixtures partially enriched with specific diastereomers are within the scope of the present invention. It will be further appreciated by the skilled artisan that substitution of the tropane ring can be in either endo- or exo-configuration, and the present invention covers both configurations. The present invention includes all the individual stereoisomers (e.g. enantiomers), racemic mixtures or partially resolved mixtures of the compounds of formulae I and, where appropriate, the individual tautomeric forms thereof.

The racemates can be used as such or can be resolved into their individual isomers. The resolution can afford stereochemically pure compounds or mixtures enriched in one or more isomers. Methods for separation of isomers are well known (cf. Allinger N. L. and Eliel E. L. in “Topics in Stereochemistry”, Vol. 6, Wiley Interscience, 1971) and include physical methods such as chromatography using a chiral adsorbent. Individual isomers can be prepared in chiral form from chiral precursors. Alternatively individual isomers can be separated chemically from a mixture by forming diasteromeric salts with a chiral acid, such as the individual enantiomers of 10-camphorsulfonic acid, camphoric acid, .alpha.-bromocamphoric acid, tartaric acid, diacetyltartaric acid, malic acid, pyrrolidone-5-carboxylic acid, and the like, fractionally crystallizing the salts, and then freeing one or both of the resolved bases, optionally repeating the process, so as obtain either or both substantially free of the other; i.e., in a form having an optical purity of >95%. Alternatively the racemates can be covalently linked to a chiral compound (auxilIary) to produce diastereomers which can be separated by chromatography or by fractional crystallization after which time the chiral auxiliary is chemically removed to afford the pure enantiomers.

The compounds of formula I contain at least one basic center and suitable acid addition salts are formed from acids which form non-toxic salts. Examples of salts of inorganic acids include the hydrochloride, hydrobromide, hydroiodide, chloride, bromide, iodide, sulphate, bisulphate, nitrate, phosphate, hydrogen phosphate. Examples of salts of organic acids include acetate, fumarate, pamoate, aspartate, besylate, carbonate, bicarbonate, camsylate, D and L-lactate, D and L-tartrate, esylate, mesylate, malonate, orotate, gluceptate, methylsulphate, stearate, glucuronate, 2-napsylate, tosylate, hibenzate, nicotinate, isethionate, malate, maleate, citrate, gluconate, succinate, saccharate, benzoate, esylate, and pamoate salts. For a review on suitable salts see Berge et al, J. Pharm. Sci., 66, 1-19, 1977.

The term “solvate” as used herein means a compound of the invention or a salt, thereof, that further includes a stoichiometric or non-stoichiometric amount of a solvent bound by non-covalent intermolecular forces. Preferred solvents are volatile, non-toxic, and/or acceptable for administration to humans in trace amounts.

The term “hydrate” as used herein means a compound of the invention, or a salt thereof, that further includes a stoichiometric or non-stoichiometric amount of water bound by non-covalent intermolecular forces.

The term “clathrate” as used herein means a compound of the invention or a salt thereof in the form of a crystal lattice that contains spaces (e.g., channels) that have a guest molecule (e.g., a solvent or water) trapped within.

The term “nucleoside and nucleotide reverse transcriptase inhibitors” (“NRTI”s) as used herein means nucleosides and nucleotides and analogues thereof that inhibit the activity of HIV-1 reverse transcriptase, the enzyme which catalyzes the conversion of viral genomic HIV-1 RNA into proviral HIV-1 DNA. Typical suitable NRTIs include zidovudine (AZT) available as RETROVIR® from Glaxo-Wellcome Inc.; didanosine (ddl) available as VIDEX® from Bristol-Myers Squibb Co.; zalcitabine (ddC) available as HIVID® from Roche Pharmaceuticals; stavudine (d4T) available as ZERIT® from Bristol-Myers Squibb Co.; lamivudine (3TC) available as EPIVIR® from Glaxo-Wellcome; abacavir (1592U89) disclosed in WO96/30025 and available ZIAGEN® from Glaxo-Wellcome; adefovir dipivoxil [bis(POM)-PMEA] available as PREVON® from Gilead Sciences; lobucavir (BMS-180194), a nucleoside reverse transcriptase inhibitor disclosed in EP-0358154 and EP-0736533 and under development by Bristol-Myers Squibb; BCH-10652, a reverse transcriptase inhibitor (in the form of a racemic mixture of BCH-10618 and BCH-10619) under development by Biochem Pharma; emitricitabine [(−)-FTC] licensed from Emory University under U.S. Pat. No. 5,814,639 and under development by Triangle Pharmaceuticals; beta-L-FD4 (also called beta-L-D4C and named beta-L-2′,3′-dicleoxy-5-fluoro-cytidene) licensed by Yale University to Vion Pharmaceuticals; DAPD, the purine nucleoside, (−)-b-D-2,6-diamino-purine dioxolane disclosed in EP-0656778 and licensed by Emory University and the University of Georgia to Triangle Pharmaceuticals; and lodenosine (FddA), 9-(2,3-dideoxy-2-fluoro-b-D-threo-pentofuranosyl)adenine, an acid stable purine-based reverse transcriptase inhibitor discovered by the NIH and under development by U.S. Bioscience Inc.

The term “non-nucleoside reverse transcriptase inhibitors” (“NNRTI”s) as used herein means non-nucleosides that inhibit the activity of HIV-1 reverse transcriptase. Typical suitable NNRTIs include nevirapine (BI-RG-587) available as VIRAMUNE® from Roxane Laboratories; delaviradine (BHAP, U-90152) available as RESCRIPTOR® from Pfizer; efavirenz (DMP-266) a benzoxazin-2-one disclosed in WO94/03440 and available as SUSTIVA® from Bristol-Myers Squibb Co.; PNU-142721, a furopyridine-thio-pyrimide under development by Pfizer 08807; AG-1549 (formerly Shionogi # S-1153); 5-(3,5-dichlorophenyl)-thio-4-isopropyl-1-(4-pyridyl)methyl-1H-imidazol-2-ylmethyl carbonate disclosed in WO 96/10019 and under development by Agouron Pharmaceuticals, Inc.; MKC-442 (1-(ethoxy-methyl)-5-(1-methylethyl)-6-(phenylmethyl)-(2,4(1H,3H)-pyrimidinedione) discovered by Mitsubishi Chemical Co. and under development by Triangle Pharmaceuticals; and (+)-calanolide A (NSC-675451) and B, coumarin derivatives disclosed in NIH U.S. Pat. No. 5,489,697, licensed to Med Chem Research, which is co-developing (+) calanolide A with Vita-invest as an orally administrable product.

The term “protease inhibitor” (“PI”) as used herein means inhibitors of the HIV-1 protease, an enzyme required for the proteolytic cleavage of viral polyprotein precursors (e.g., viral GAG and GAG Pol polyproteins), into the individual functional proteins found in infectious HIV-1. HIV protease inhibitors include compounds having a peptidomimetic structure, high molecular weight (7600 daltons) and substantial peptide character. Typical suitable PIs include saquinavir (Ro 31-8959) available in hard gel capsules as INVIRASE® and as soft gel capsules as FORTOVASE® from Roche Pharmaceuticals, Nutley, N.J. 07110-1199; ritonavir (ABT-538) available as NORVIR® from Abbott Laboratories; indinavir (MK-639) available as CRIXIVAN® from Merck & Co., Inc.; nelfnavir (AG-1343) available VIRACEPT® from Agouron Pharmaceuticals, Inc.; amprenavir (141W94), AGENERASE®, a non-peptide protease inhibitor under development by Vertex Pharmaceuticals, Inc. and available from Glaxo-Wellcome, under an expanded access program; lasinavir (BMS-234475) available from Bristol-Myers Squibb; DMP-450, a cyclic urea discovered by Dupont and under development by Triangle Pharmaceuticals; BMS-2322623, an azapeptide under development by Bristol-Myers Squibb as a 2nd-generation HIV-1 PI; ABT-378 under development by Abbott; and AG-1549 an orally active imidazole carbamate discovered by Shionogi and under development by Agouron Pharmaceuticals, Inc.

Other antiviral agents include hydroxyurea, ribavirin, IL-2, IL-12, pentafuside. Hydroyurea (Droxia), a ribonucleoside triphosphate reductase inhibitor, the enzyme involved in the activation of T-cells, was discovered at the NCI and is in preclinical studies, it was shown to have a synergistic effect on the activity of didanosine and has been studied with stavudine. IL-2 is disclosed in Ajinomoto EP-0142268, Takeda EP-0176299, and Chiron U.S. Pat. Nos. Re. 33,653, 4,530,787, 4,569,790, 4,604,377, 4,748,234, 4,752,585, and 4,949,314, and is available under the PROLEUKIN® (aldesleukin) as a lyophilized powder for IV infusion or sc administration upon reconstitution and dilution with water; a dose of about 1 to about 20 million 1 U/day, sc is preferred; a dose of about 15 million 1 U/day, sc is more preferred. IL-12 is disclosed in WO96/25171 and is administered in a dose of about 0.5 microgram/kg/day to about 10 microgram/kg/day, sc is preferred. Pentafuside (FUZEON®) a 36-amino acid synthetic peptide, disclosed in U.S. Pat. No. 5,464,933 that acts by inhibiting fusion of HIV-1 to target membranes. Pentafuside (3-100 mg/day) is given as a continuous sc infusion or injection together with efavirenz and 2 PI's to HIV-1 positive patients refractory to a triple combination therapy; use of 100 mg/day is preferred. Ribavirin, 1-β-D-ribofuranosyl-1H-1,2,4-triazole-3-carboxamide, is available from ICN Pharmaceuticals, Inc., Costa Mesa, Calif.; its manufacture and formulation are described in U.S. Pat. No. 4,211,771.

The term “viral fusion inhibitors” as used herein refers to compounds which inhibit fusion of the free virus particle and introduction of the viral RNA into a host cell independent of the molecular locus of inhibitor binding. Viral fusion inhibitors therefore include, but are not limited to T-20; CD-4 binding ligands including BMS-378806, BMS-488043; CCR5 binding ligands including Sch-351125, Sch-350634, Sch-417690 (Schering Plough), UK-4278957 (Pfizer), TAK-779 (Takeda), ONO-4128 (Ono), AK-602 (Ono, GlaxoSmithKline), compounds 1-3 (Merck); CXCR4 binding ligands KRH-1636 (K. Ichiyama et al. Proc. Nat. Acad. Sci USA 2003 100(7):4185-4190), T-22 (T. Murakami et al. J. Virol. 1999 73(9):7489-7496), T-134 (R. Arakaki et al. J. Virol. 1999 73(2):1719-1723). Viral fusion inhibitors as used herein also include peptide and protein soluble receptors, antibodies, chimeric antibodies, humanized antibodies.

Commonly used abbreviations include: acetyl (Ac), azo-bis-isobutyrylnitrile (AIBN), atmospheres (Atm), 9-borabicyclo[3.3.1]nonane (9-BBN or BBN), tert-butoxycarbonyl (Boc), di-tert-butyl pyrocarbonate or boc anhydride (BOC₂O), benzyl (Bn), butyl (Bu), benzyloxycarbonyl (CBZ or Z), Chemical Abstracts Registry Number (CAS Reg. No.), carbonyl diimidazole (CDI), 1,4-diazabicyclo[2.2.2]octane (DABCO), diethylaminosulfur trifluoride (DAST), dibenzylideneacetone (dba), 1,5-diazabicyclo[4.3.0]non-5-ene (DBN), 1,8-diazabicyclo[5.4.0]undec-7-ene (DBU), N,N′-dicyclohexylcarbodiimide (DCC), 1,2-dichloroethane (DCE), dichloromethane (DCM), diethyl azodicarboxylate (DEAD), di-iso-propylazodicarboxylate (DIAD), di-iso-butylaluminumhydride (DIBAL or DIBAL-H), di-iso-propylethylamine (DIPEA), N,N-dimethyl acetamide (DMA), 4-N,N-dimethylaminopyridine (DMAP), N,N-dimethylformamide (DMF), dimethyl sulfoxide (DMSO), (diphenylphosphino)ethane (dppe), (diphenylphosphino)ferrocene (dppf), 1-(3-dimethylaminopropyl)-3-ethylcarbodiimide hydrochloride (EDCI), ethyl (Et), ethyl acetate (EtOAc), ethanol (EtOH), 2-ethoxy-2H-quinoline-1-carboxylic acid ethyl ester (EEDQ), diethyl ether (Et₂O), acetic acid (HOAc), 1-N-hydroxybenzotriazole (HOBt), high pressure liquid chromatography (HPLC), lithium hexamethyl disilazane (LiHMDS), methanol (MeOH), melting point (mp), MeSO₂— (mesyl or Ms), methyl (Me), acetonitrile (MeCN), m-chloroperbenzoic acid (MCPBA), mass spectrum (ms), methyl t-butyl ether (MTBE), N-bromosuccinimide (NBS), N-carboxyanhydride (NCA), N-chlorosuccinimide (NCS), N-methylmorpholine (NMM), N-methylpyrrolidone (NMP), pyridinium chlorochromate (PCC), pyridinium dichromate (PDC), phenyl (Ph), propyl (Pr), iso-propyl (i-Pr), pounds per square inch (psi), pyridine (pyr), room temperature (rt or RT), tert-butyldimethylsilyl or t-BuMe₂Si (TBDMS), triethylamine (TEA or Et₃N), triflate or CF₃SO₂— (Tf), trifluoroacetic acid (TFA), 1,1′-bis-2,2,6,6-tetramethylheptane-2,6-dione (TMHD), O-benzotriazol-1-yl-N,N,N′,N′-tetramethyluronium tetrafluoroborate (TBTU), 1,1′-bis-thin layer chromatography (TLC), tetrahydrofuran (THF), trimethylsilyl or Me₃Si (TMS), p-toluenesulfonic acid monohydrate (TsOH or pTsOH), 4-Me-C₆H₄SO₂— or tosyl (Ts), N-urethane-N-carboxyanhydride (UNCA), Conventional nomenclature including the prefixes normal (n), iso (i-), secondary (sec-), tertiary (tert-) and neo have their customary meaning when used with an alkyl moiety. (J. Rigaudy and D. P. Klesney, Nomenclature in Organic Chemistry, IUPAC 1979 Pergamon Press, Oxford.).

### Compounds and Preparation

Compounds of the present invention can be made by a variety of methods depicted in the illustrative synthetic reaction schemes shown and described below. The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, Volumes 1-21; R. C. LaRock, Comprehensive Organic Transformations, 2^{nd} edition Wiley-VCH, New York 1999; Comprehensive Organic Synthesis, B. Trost and I. Fleming (Eds.) vol. 1-9 Pergamon, Oxford, 1991; Comprehensive Heterocyclic Chemistry, A. R. Katritzky and C. W. Rees (Eds) Pergamon, Oxford 1984, vol. 1-9; Comprehensive Heterocyclic Chemistry II, A. R. Katritzky and C. W. Rees (Eds) Pergamon, Oxford 1996, vol. 1-11; and Organic Reactions, Wiley & Sons: New York, 1991, Volumes 1-40. The following synthetic reaction schemes are merely illustrative of some methods by which the compounds of the present invention can be synthesized, and various modifications to these synthetic reaction schemes can be made and will be suggested to one skilled in the art having referred to the disclosure contained in this Application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein preferably are conducted under an inert atmosphere at atmospheric pressure at a reaction temperature range of from about −78° C. to about 150° C., more preferably from about 0° C. to about 125° C., and most preferably and conveniently at about room (or ambient) temperature, e.g., about 20° C.

2-Benzyl-octahydro-pyrrolo[3,4-c]pyrrole (11a) was prepared by [2,3]-dipolar cycloaddition of an imine ylide with N-benzylmaleimide as described previously (R. Colon-Cruz et al. WO 02/070523 and M. Björsne et al. WO 02/060902). Reduction of the imide, and selective debenzylation are accomplished as described therein. Pyrrolo[3,4-c]pyrrole-2(1H)-carboxylic acid, hexahydro-, 1,1-dimethylethyl ester (11b) is prepared from 11a by acylation and debenzylation (R. Colon-Cruz et al. WO 02/070523, supra).

Compounds of the present invention are prepared by step-wise elaboration of a protected octahydro-pyrrolo[3,4-c]pyrrole (11) as generally depicted in SCHEME 1. In SCHEME 1 R¹-R⁴ and X¹ are as defined 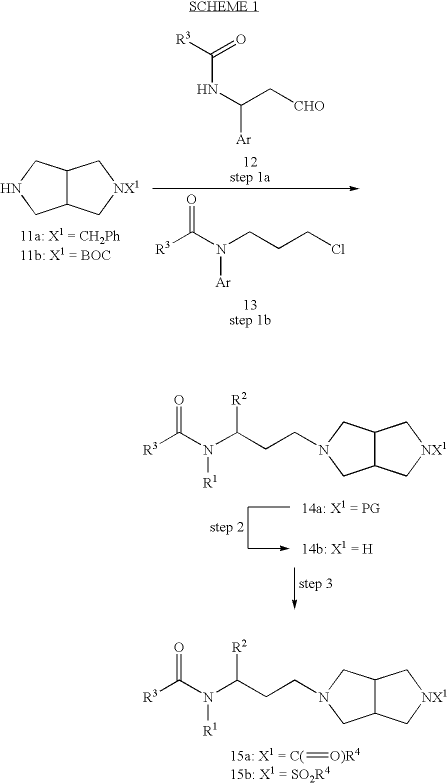
in SCHEME 1 and claim 1 and Ar represents optionally substituted phenyl represented by R¹ or R² in claim 1. Compounds of the present invention wherein R² is phenyl and R¹ is hydrogen are typically prepared by reductive amination of 11a or 11b with a β-amino aldehyde 12 as depicted in step 1a to afford 14a wherein R² is aryl. Compounds of the present invention wherein R¹ is phenyl and R² is hydrogen are typically prepared by alkylation of 11a or 11b with an alkyl halide 13 as depicted in step 1b to afford 14a wherein R¹ is aryl. After the first nitrogen substituent is introduced the protecting group is removed to afford 14b and the second nitrogen is acylated to afford 15a or sulfonylated to afford 15b. One skilled in the art will appreciate that the sequence of these steps can be reversed such that the acyl/sulfonylation is carried out first on 11a or 11b and the reductive amination/alkylation is carried out after removal of the nitrogen protecting group.

Reductive amination is preferably carried out carried out by combining an amine and carbonyl compound in the presence of a complex metal hydride such as sodium borohydride, lithium borohydride, sodium cyanoborohydride, zinc borohydride, sodium triacetoxyborohydride or borane/pyridine conveniently at a pH of 1-7 or with hydrogen in the presence of a hydrogenation catalyst, e.g. in the presence of palladium/charcoal, at a hydrogen pressure of 1 to 5 bar, preferably at temperatures between 20° C. and the boiling temperature of the solvent used. Optionally a dehydrating agent, such as molecular sieves or Ti(IV)(O-i-Pr)₄, is added to facilitate formation of the intermediate imine at ambient temperature. It may also be advantageous to protect potentially reactive groups during the reaction with conventional protecting groups which are cleaved again by conventional methods after the reaction. Reductive amination procedures have been reviewed: R. M. Hutchings and M. K. Hutchings Reduction of C═N to CHNH by Metal Hydrides in Comprehensive Organic Synthesis col. 8, I. Fleming (Ed) Pergamon, Oxford 1991 pp. 47-54.

The amine alkylation is accomplished by treating the amine or a metal salt of the amine (i.e. a deprotonated form) with a compound RZ¹ wherein Z¹ is a leaving group such as halo, C₁₋₄ alkanesulphonyloxy, benzenesulphonyloxy or p-toluenesulphonyloxy. In the example depicted in SCHEME 1 RZ¹ is 13 and Z¹ is chloride. The reaction is optionally carried out in the presence of a base and/or a phase transfer catalyst. Commonly used bases include, but are not limited to, triethylamine, N,N-diisopropylethylamine or DBU; or an inorganic base such as Na₂CO₃, NaHCO₃, K₂CO₃ or Cs₂CO₃. Commonly used solvents include, but are not limited to acetonitrile, DMF, DMSO, 1,4-dioxane, THF or toluene. The reaction is conveniently run by in the presence of NaI which forms a more reactive intermediate alkyl iodide (Z¹ is iodide).

The acylation is conveniently carried out with a corresponding acyl halide or acid anhydride in a solvent such as DCM, chloroform, carbon tetrachloride, ether, THF, dioxane, benzene, toluene, MeCN, DMF, aqueous sodium hydroxide solution or sulfolane optionally in the presence of an inorganic or organic base at temperatures between −20 and 200° C., but preferably at temperatures between −10 and 100° C. Typical organic bases include tertiary amines include but are not limited to TEA, pyridine. Typical inorganic bases include but are not limited to K₂CO₃ and NaHCO₃.

The acylation may however also be carried out with the free acid optionally in the presence of an acid-activating agent or a dehydrating agent, e.g. isobutyl chloroformate, carbodiimides such as EDCI or DCC optionally in the presence of an additive such as HOBt or N-hydroxysuccinimide, O-(benzotriazol-1-yl)-N,N,N′,N′-tetramethyl-uronium tetrafluoroborate (TBTU) in the presence of a base such as DIPEA or NMM, N,N′-carbonyldiimidazole, N,N′-thionyldiimidazole or triphenylphosphine/carbon tetrachloride, at temperatures between −20 and 200° C., but preferably at temperatures between −10 and 100° C.

A sulfonylation maybe conveniently carried out by treating the amine with a sulfonyl chloride in a solvent such as DCM, chloroform, carbon tetrachloride, ether, THF, dioxane, benzene, toluene, MeCN, DMF, aqueous sodium hydroxide solution or sulfolane in the presence of an organic base such as amines which include but are not limited to TEA, pyridine at temperatures between −10 and 120° C.

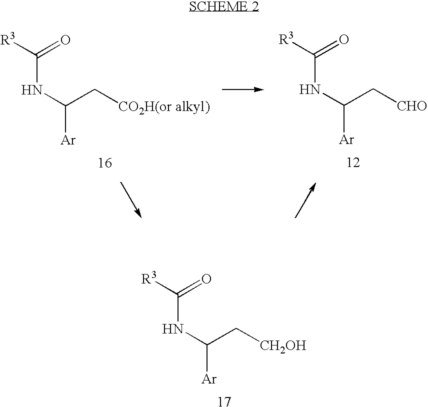

The β-acylamino aldehyde 12 can be prepared by direct reduction of a β-acylamino acid 16 or ester with a hydride reducing agent such as DIBAL-H or 16 can be reduced to the corresponding alcohol and re-oxidized to the aldehyde with a SO₃-pyridine and TEA or alternative oxidizing agent. The R³ moiety can either be the moiety present in the final compound or alternatively R³C(═O) can be a protecting group, e.g., R³=O-tert-Bu (BOC), which can be removed to liberate a primary amine at an advantageous stage in the synthetic sequence which can be acylated or sulfonylated as desired. Compounds according to formula I wherein R² is optionally substituted phenyl are generally conveniently prepared utilizing the BOC protecting group as R³C(═O). Deprotection and acylation is conveniently carried out in the latter stage of the synthesis. The specific acylating agents used are described in the following examples.

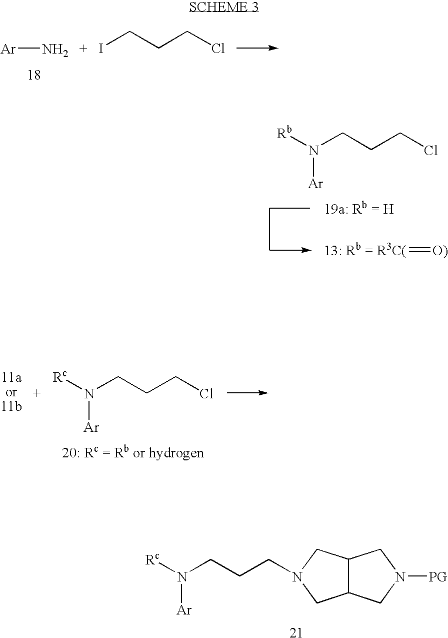

The 3-chloro-propyl-N-aryl-amine 19a is prepared by alkylation of an optionally substituted aniline 18 with 3-iodo-1-chloro-propane. Alkylation of 11a or 11b can be accomplished with the secondary amine 19a which is subsequently acylated; or, alternatively the secondary amine can be acylated to afford 13 wherein R³ is as defined in claim 1 or R³C(═O) is a protecting group removed prior to the acylation step. Removal of the protecting group (PG) of 11 and acylation or sulfonylation as described above affords 15a or 15b respectively wherein R¹ is aryl and R² is hydrogen.

A generally useful sequence to prepare compounds contemplated in the invention wherein R¹ is optionally substituted phenyl (15a; R¹=aryl; R²=H) is to introduce the desired X¹ moiety (11; X¹=C(═O)R³ or SO₂R³) by acylation of 11a or 11b followed deprotection and alkylation with 20 (R^{c}=H). Acylation of the resulting secondary amine with carboxylic acids (in the presence of dehydrating agents such as DCC or EDCI) or carboxylic acid derivatives can be carried out by procedures described above and in the following examples. The carboxylic acids or carboxylic acid derivatives are commercially available or prepared by known procedures which are exemplified in the following examples. The term “carboxylic acid derivatives” as used herein refers to acid halides, esters and anhydrides. Similarly, β-tert-butoxycarbonyl-β-aryl-propanal 12 (R³=tert-BuO) derivatives can be conveniently linked to 11 (X¹=C(═O)R³ or SO₂R³) by reductive amination to prepare compounds of the invention wherein R¹ is hydrogen and R² is optionally substituted aryl. Removal of the BOC protecting group and acylation of the amine as described above affords a flexible and efficient route to compounds described herein.

Examples of representative compounds encompassed by the present invention and within the scope of the invention are provided in the following Tables. These examples and preparations which follow are provided to enable those skilled in the art to more clearly understand and to practice the present invention. They should not be considered as limiting the scope of the invention, but merely as being illustrative and representative thereof.

In general, the nomenclature used in this Application is based on AUTONOM™ v.4.0, a Beilstein Institute computerized system for the generation of IUPAC systematic nomenclature. If there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

TABLE I

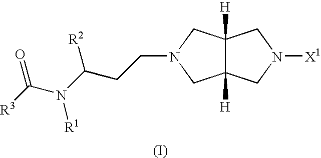

| Cpd. | | | | | | |
|---|---|---|---|---|---|---|
| No. | R¹ | R² | R³ | X¹ | ms | salt |
| I-1 | Cl-Me- | H | HO₂C(CH₂)₃NH | A | 557.3 | |
| | Ph | | | | | |
| I-2 | Cl-Me- | H | 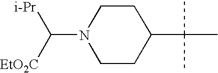 | A | 667.4 | TFA |
| I-3 | Cl-Me- Ph | H | 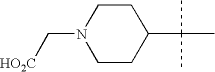 | A | 597.3 | TFA |
| I-4 | Cl-Me- Ph | H | 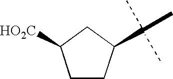 | C | 566.3 | TFA |
| I-5 | Cl-Me- Ph | H | 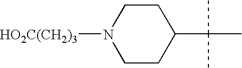 | A | 625.4 | TFA |
| I-6 | Cl-Me- Ph | H | 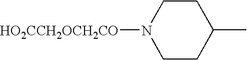 | A | 655.4 | TFA |
| I-7 | Cl-Me- Ph | H | 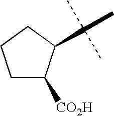 | A | 568.2 | TFA |
| I-8 | Cl-Me- Ph | H | HO₂C(CH₂)₂NH | A | 543.4 | TFA |
| I-9 | H | m-F-C₆H₃ | 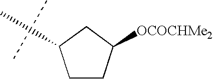 | A | 580 | |
| I-10 | Cl-Me- Ph | H | 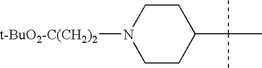 | A | 668.4 | TFA |
| I-11 | Cl-Me- Ph | H | 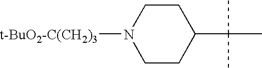 | A | 681.3 | TFA |
| I-12 | Cl-Me- Ph | H | 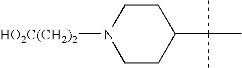 | A | 611.4 | TFA |
| I-13 | H | Ph | HO₂CCH(i-Pr) | C | 506 | TFA |
| I-14 | H | Ph | HO₂CCH(Cy) | C | 546 | TFA |
| I-12 | Cl-Me- Ph | H | 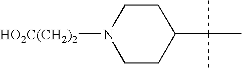 | A | 6 | |
| I-15 | Cl-Me- Ph | H | 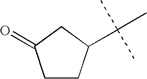 | A | 538 | TFA |
| I-16 | Ph | H | 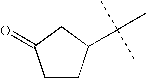 | A | 490 | TFA |
| I-17 | H | m-F-C₆H₃ | 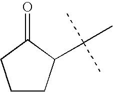 | A | 508 | |
| I-18 | Cl-Me- Ph | H | 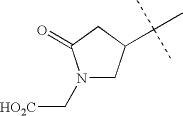 | A | 597 | TFA |
| I-19 | Cl-Me- Ph | H | 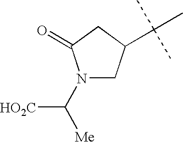 | A | 611 | TFA |
| I-20 | H | Ph | MeO₂CCH(Cy) | C | 560 | |
| I-21 | H | m-NC- C₆H₃ | 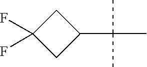 | A | 523 | |
| I-22 | H | m-NC- C₆H₃ | 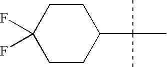 | A | 551 | |
| I-23 | H | m-F-C₆H₄ | 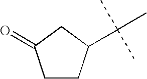 | B | 507 | |
| I-24 | H | m-F-C₆H₄ | 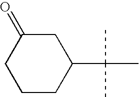 | A | 522 | |
| I-25 | Cl-Me- Ph | H | 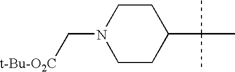 | A | 653.4 | |
| I-26 | Cl-Me- Ph | H | 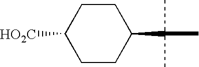 | A | 582.2 | TFA |
| I-27 | Cl-Me- Ph | H | 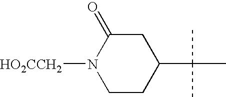 | B | 610.4 | TFA |
| I-28 | Cl-Me- Ph | H | 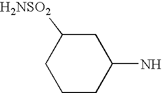 | A | 626, 628 | |
| I-29 | Cl-Me- Ph | H | 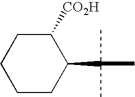 | C | 580.3 | TFA |
| I-30 | Cl-Me- Ph | H | 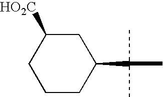 | C | 580.3 | TFA |
| I-31 | Cl-Me- Ph | H | 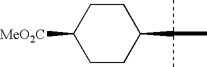 | A | 596.4 | TFA |
| I-32 | Cl-Me- Ph | H | 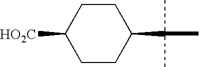 | A | 582.3 | TFA |
| I-33 | H | Ph | 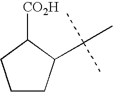 | C | 518 | |
| I-34 | H | Ph | HO₂CCH₂NH | C | 479 | |
| I-35 | H | Ph | HO₂C(CH₂)₃NH | C | 507 | |
| I-36 | H | Ph | 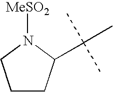 | A | 555 | |
| I-37 | H | Ph | 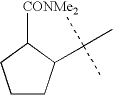 | A | 547.5 | TFA |
| I-38 | H | 3-F-C₆H₄ | 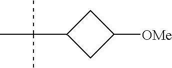 | A | 510 | |
| I-39 | H | 3-F-C₆H₄ | 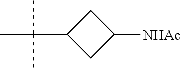 | A | 537 | |
| I-40 | H | 3-F-C₆H₄ | 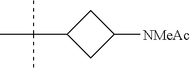 | A | 551 | |
| I-41 | H | 3-F-C₆H₄ | 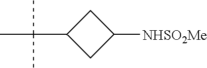 | A | 573 | TFA |
| I-42 | H | 3-F-C₆H₄ | 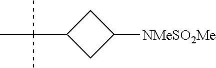 | A | 587 | TFA |
| I-43 | H | 3-F-C₆H₄ | 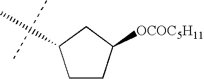 | A | 608 | TFA |

Cyp = cyclopentyl
A = CO-4,6-diMe-pyrimidin-5-yl
B = CO-2,4diMe-pyridin-3-yl
C = CO-2,6-dimethyl-phenyl
Cl-Me-Ph = 3-Cl-4-Me-C₆H₃

Other representative compounds within the scope of the present invention are depicted in TABLE 2. A more detailed description of X¹ groups is contained in the U.S. patent application filed herewith entitled Heterocyclic Antiviral compounds which is herein incorporated by reference in it entirety. One skilled in the art will appreciated that the synthetic methods disclosed herein can be readily adapted to other X¹ and R³ groups.

TABLE 2

| Cpd. | |
|---|---|
| No. | Structure |
| II-1 | 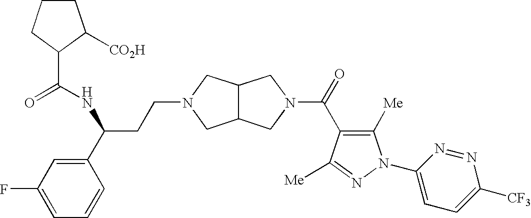 |
| II-2 | 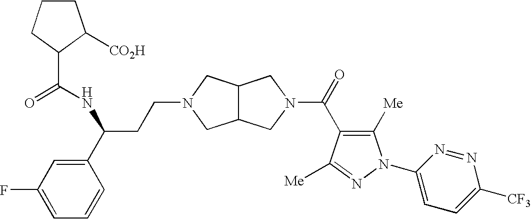 |
| II-3 | 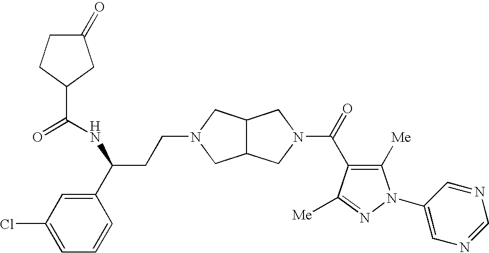 |
| II-4 | 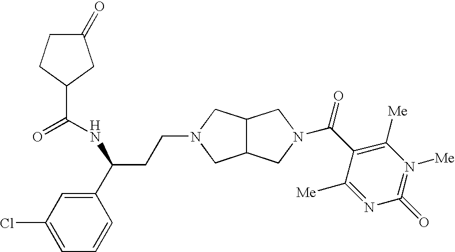 |
| II-5 | 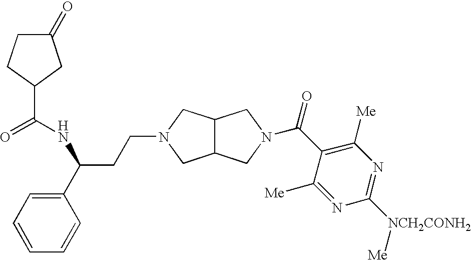 |
| II-6 | 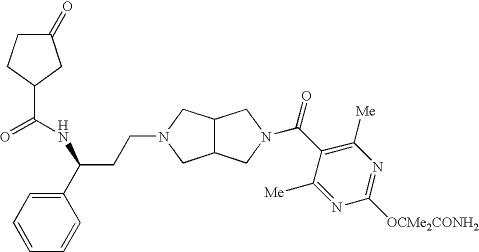 |
| II-7 | 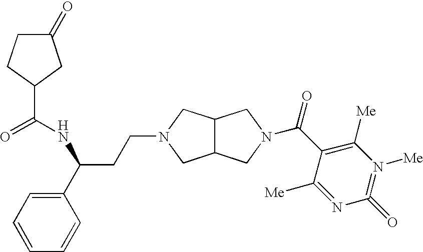 |
| II-8 | 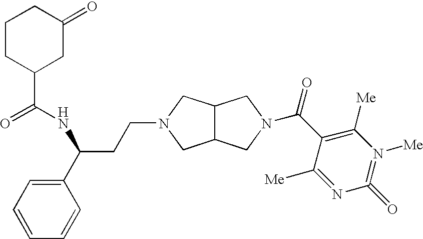 |

### Dosage and Administration

The compounds of the present invention may be formulated in a wide variety of oral administration dosage forms and carriers. Oral administration can be in the form of tablets, coated tablets, dragees, hard and soft gelatine capsules, solutions, emulsions, syrups, or suspensions. Compounds of the present invention are efficacious when administered by other routes of administration including continuous (intravenous drip) topical parenteral, intramuscular, intravenous and suppository administration, among other routes of administration. The preferred manner of administration is generally oral using a convenient daily dosing regimen which can be adjusted according to the degree of affliction and the patient's response to the active ingredient.

A compound or compounds of the present invention, as well as their pharmaceutically useable salts, together with one or more conventional excipients, carriers, or diluents, may be placed into the form of pharmaceutical compositions and unit dosages. The pharmaceutical compositions and unit dosage forms may be comprised of conventional ingredients in conventional proportions, with or without additional active compounds or principles, and the unit dosage forms may contain any suitable effective amount of the active ingredient commensurate with the intended daily dosage range to be employed. The pharmaceutical compositions may be employed as solids, such as tablets or filled capsules, semisolids, powders, sustained release formulations, or liquids such as solutions, suspensions, emulsions, elixirs, or filled capsules for oral use; or in the form of suppositories for rectal or vaginal administration; or in the form of sterile injectable solutions for parenteral use. A typical preparation will contain from about 5% to about 95% active compound or compounds (w/w). The term “preparation” or “dosage form” is intended to include both solid and liquid formulations of the active compound and one skilled in the art will appreciate that an active ingredient can exist in different preparations depending on the target organ or tissue and on the desired dose and pharmacokinetic parameters.

The term “excipient” as used herein refers to a compound that is useful in preparing a pharmaceutical composition, generally safe, non-toxic and neither biologically nor otherwise undesirable, and includes excipients that are acceptable for veterinary use as well as human pharmaceutical use. The compounds of this invention can be administered alone but will generally be administered in admixture with one or more suitable pharmaceutical excipients, diluents or carriers selected with regard to the intended route of administration and standard pharmaceutical practice.

A “pharmaceutically acceptable salt” form of an active ingredient may also initially confer a desirable pharmacokinetic property on the active ingredient which were absent in the non-salt form, and may even positively affect the pharmacodynamics of the active ingredient with respect to its therapeutic activity in the body. The phrase “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. It should be understood that all references to pharmaceutically acceptable salts include solvent addition forms (solvates) or crystal forms (polymorphs) as defined herein, of the same acid addition salt.

Solid form preparations include powders, tablets, pills, capsules, cachets, suppositories, and dispersible granules. A solid carrier may be one or more substances which may also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. Suitable carriers include but are not limited to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Solid form preparations may contain, in addition to the active component, colorants, flavors, stabilizers, buffers, artificial and natural sweeteners, dispersants, thickeners, solubilizing agents, and the like.

Liquid formulations also are suitable for oral administration include liquid formulation including emulsions, syrups, elixirs, aqueous solutions, aqueous suspensions. These include solid form preparations which are intended to be converted to liquid form preparations shortly before use. Emulsions may be prepared in solutions, for example, in aqueous propylene glycol solutions or may contain emulsifying agents such as lecithin, sorbitan monooleate, or acacia. Aqueous solutions can be prepared by dissolving the active component in water and adding suitable colorants, flavors, stabilizing, and thickening agents. Aqueous suspensions can be prepared by dispersing the finely divided active component in water with viscous material, such as natural or synthetic gums, resins, methylcellulose, sodium carboxymethylcellulose, and other well known suspending agents.

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

The compounds of the present invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The compounds of the present invention may be formulated for vaginal administration. Pessaries, tampons, creams, gels, pastes, foams or sprays containing in addition to the active ingredient such carriers as are known in the art to be appropriate.

When desired, formulations can be prepared with enteric coatings adapted for sustained or controlled release administration of the active ingredient. For example, the compounds of the present invention can be formulated in transdermal or subcutaneous drug delivery devices. These delivery systems are advantageous when sustained release of the compound is necessary and when patient compliance with a treatment regimen is crucial. Compounds in transdermal delivery systems are frequently attached to an skin-adhesive solid support. The compound of interest can also be combined with a penetration enhancer, e.g., Azone (1-dodecylaza-cycloheptan-2-one). Sustained release delivery systems are inserted subcutaneously into to the subdermal layer by surgery or injection. The subdermal implants encapsulate the compound in a lipid soluble membrane, e.g., silicone rubber, or a biodegradable polymer, e.g., polyactic acid.

Suitable formulations along with pharmaceutical carriers, diluents and expcipients are described in Remington: The Science and Practice of Pharmacy 1995, edited by E. W. Martin, Mack Publishing Company, 19th edition, Easton, Pa. A skilled formulation scientist may modify the formulations within the teachings of the specification to provide numerous formulations for a particular route of administration without rendering the compositions of the present invention unstable or compromising their therapeutic activity.

The modification of the present compounds to render them more soluble in water or other vehicle, for example, may be easily accomplished by minor modifications (salt formulation, esterification, etc.), which are well within the ordinary skill in the art. It is also well within the ordinary skill of the art to modify the route of administration and dosage regimen of a particular compound in order to manage the pharmacokinetics of the present compounds for maximum beneficial effect in patients.

The term “therapeutically effective amount” as used herein means an amount required to reduce symptoms of the disease in an individual. The dose will be adjusted to the individual requirements in each particular case. That dosage can vary within wide limits depending upon numerous factors such as the severity of the disease to be treated, the age and general health condition of the patient, other medicaments with which the patient is being treated, the route and form of administration and the preferences and experience of the medical practitioner involved. For oral administration, a daily dosage of between about 0.01 and about 100 mg/kg body weight per day should be appropriate in monotherapy and/or in combination therapy. A preferred daily dosage is between about 0.1 and about 500 mg/kg body weight, more preferred 0.1 and about 100 mg/kg body weight and most preferred 1.0 and about 10 mg/kg body weight per day. Thus, for administration to a 70 kg person, the dosage range would be about 7 mg to 0.7 g per day. The daily dosage can be administered as a single dosage or in divided dosages, typically between 1 and 5 dosages per day. Generally, treatment is initiated with smaller dosages which are less than the optimum dose of the compound. Thereafter, the dosage is increased by small increments until the optimum effect for the individual patient is reached. One of ordinary skill in treating diseases described herein will be able, without undue experimentation and in reliance on personal knowledge, experience and the disclosures of this application, to ascertain a therapeutically effective amount of the compounds of the present invention for a given disease and patient.

In embodiments of the invention, the active compound or a salt can be administered in combination with another antiviral agent, such as a nucleoside reverse transcriptase inhibitor, another non-nucleoside reverse transcriptase inhibitor or HIV protease inhibitor. When the active compound or its derivative or salt are administered in combination with another antiviral agent the activity may be increased over the parent compound. When the treatment is combination therapy, such administration may be concurrent or sequential with respect to that of the nucleoside derivatives. “Concurrent administration” as used herein thus includes administration of the agents at the same time or at different times. Administration of two or more agents at the same time can be achieved by a single formulation containing two or more active ingredients or by substantially simultaneous administration of two or more dosage forms with a single active agent.

It will be understood that references herein to treatment extend to prophylaxis as well as to the treatment of existing conditions, and that the treatment of animals includes the treatment of humans as well as other primates. Furthermore, treatment of a HIV infection, as used herein, also includes treatment or prophylaxis of a disease or a condition associated with or mediated by HIV infection, or the clinical symptoms thereof.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The following examples illustrate the preparation and biological evaluation of compounds within the scope of the invention. These examples and preparations which follow are provided to enable those skilled in the art to more clearly understand and to practice the present invention. They should not be considered as limiting the scope of the invention, but merely as being illustrative and representative thereof.

### EXAMPLE 1

### 4-Hydroxy-4-methyl-cyclohexanecarboxylic acid (3-chloro-4-methyl-phenyl)-{3-[5-(4,6-dimethyl-pyrimidine-5-carbonyl)-hexahydro-pyrrolo[3,4-c]pyrrol-2-yl]-propyl}-amide trifluoroacetate

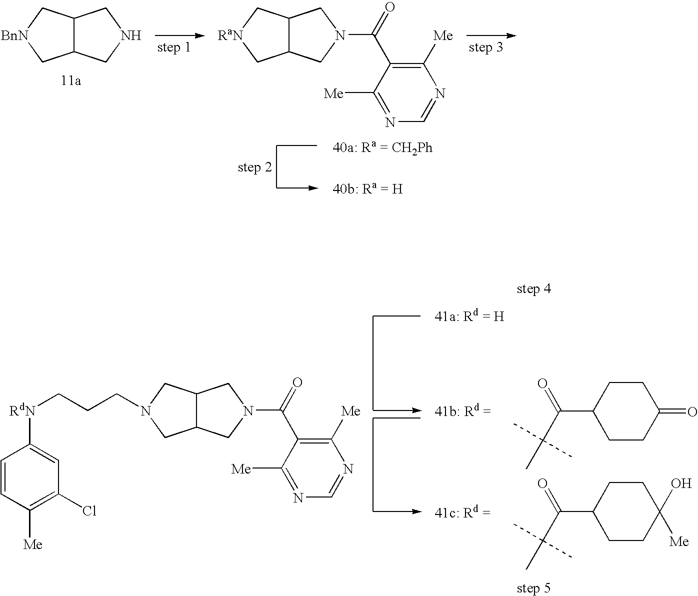 Step 1—A mixture of 2-benzyl-octahydro-pyrrolo[3,4-c]pyrrole (11a; 0.50 g, 2.47 mmol), 4,6-dimethyl-pyrimidine-5-carboxylic acid (0.44 g), EDCI (0.61 g), HOBt (0.43 g) and DIPEA (1.3 mL) in DCM (30 mL) was stirred at RT overnight. It was diluted with DCM and washed with saturated NaHCO₃. The organic layer was dried (Na₂SO₄), filtered and evaporated in vacuo. The residue was purified SiO₂ chromatography (DCM/MeOH/NH₄OH 60/10/1) to afford 0.71 g (91%) of 40a.
Step 2—A mixture of 40a (0.761 g), Pd(OH)₂ (70 mg) and ammonium formate (0.713 g) in EtOH (25 mL) was heated at reflux for several hours. The catalyst was filtered off and the filtrate was concentrated in vacuo. The residue was dissolved in MeOH and 10% Pd/C (catalytic amount) was added followed by ammonium formate (0.713 g). The reaction heated at reflux and stirred for 90 minutes then cooled to RT. The catalyst was filtered off through a CELITE® pad and the filtrate was concentrated in vacuo. The residue was purified by SiO₂ chromatography (DCM/MeOH/NH₄OH, 60/10/1) which afforded 40b.
Step 3—A mixture of 40b (0.96 g, 3.90 mmol), (3-chloro-4-methyl-phenyl)-(3-chloro-propyl)-amine (0.93 g, 4.29 mmol; prepared as described in step 1 of example except 4-chloro-3-methyl-aniline was used in place of aniline), KI (0.77 g, 4.68 mmol) and K₂CO₃ (0.80 g, 5.85 mmol) in MeCN (35 mL) was heated at reflux overnight. It was cooled at RT, diluted with water and extracted with EtOAc. The organic layer was dried (Na₂SO₄), filtered and evaporated in vacuo. The residue was purified via SiO₂ chromatography (DCM/MeOH/NH₄OH, 60/10/1) to afford 1.38 g (83%) of 41a as an oil.
Step 4—A mixture 41a (178 mg, 0.416 mmol), 4-oxo-cyclohexanecarboxylic acid (59 mg, 0.416 mmol) and EEDQ (0.10 g, 0.416 mmol) in benzene (10 mL) was heated at reflux overnight. The reaction mixture was cooled to RT and evaporated. The crude product was purified via SiO₂ chromatography (DCM/MeOH/NH₄OH, 60/10/1) to afford 41b.
Step 5—To a solution of 41b (30 mg) in a 1:1 mixture of THF/benzene (2 mL) was added dropwise at 0° C., a solution of MeMgBr (3M in Et₂O, 50 μL) in THF (1 mL). The reaction was stirred at 0° C. for 90 minutes, quenched with saturated NH₄Cl. The mixture was filtered and the filtrate was evaporated in vacuo. The residue was purified via preparative HPLC to afford 41c: M+H=568.

### EXAMPLE 2

### 4-Hydroxy-4-methyl-cyclohexanecarboxylic acid (3-chloro-4-methyl-phenyl)-{3-[5-(4,6-dimethyl-pyrimidine-5-carbonyl)-hexahydro-pyrrolo[3,4-c]pyrrol-2-yl]-propyl}-amide, trifluoroacetate salt (I-16)

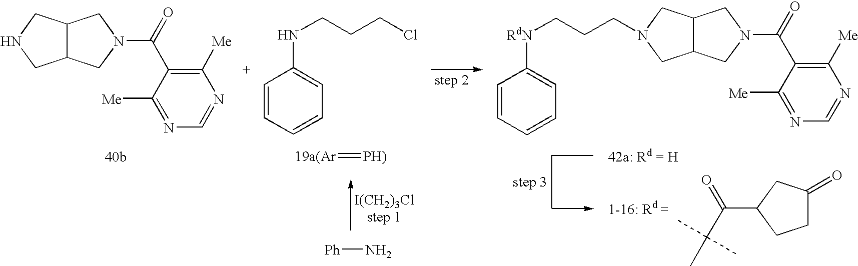 Step 1—A mixture of aniline (1 mL, 11.1 mmol), 1-chloro-3-iodo-propane (1.31 mL, 12.2 mmol) and Cs₂CO₃ (10.8 g, 33.3 mmol) in DMF (15 mL) was stirred at RT overnight. It was diluted with water and extracted with hexane. The organic layer was dried (Na₂SO₄), filtered and evaporated in vacuo. The residue was purified via SiO₂ chromatography eluting with hexane/EtOAc, (95/5) to afford 2.52 g (67%) of 19a (Ar=Ph) as an oil: M+H=170.
Step 2—A mixture of 40b (0.54 g, 2.19 mmol), (3-chloro-propyl)-phenyl-amine (19a, Ar=Ph, 0.41 g, 2.41 mmol), KI (0.54 g, 3.29 mmol) and K₂CO₃ (0.60 g, 4.38 mmol) in MeCN (15 mL) was heated at reflux overnight. The reaction mixture was cooled to RT, diluted with water and extracted with EtOAc. The combined organic layers were dried (Na₂SO₄), filtered and evaporated in vacuo. The residue was purified via SiO₂ chromatography eluting with DCM/MeOH/NH₄OH (60/10/1) to afford 0.664 g (80%) of 42 as oil: M+H=380.
Step 3—To a solution of 42 (0.0747 mmol) in benzene (1.5 mL) was added 3-oxo-1-cyclopentanecarboxylic acid (0.082 mmol) followed by EEDQ (0.089 mmol). The reaction was stirred at room temperature for 4 hours, evaporated and purified via preparative HPLC to afford the TFA salt of 1-16.

3-Oxo-cyclopentanecarboxylic acid (3-chloro-4-methyl-phenyl)-{3-[5-(4,6-dimethyl-pyrimidine-5-carbonyl)-hexahydro-pyrrolo[3,4-c]pyrrol-2-yl]-propyl}-amide, trifluoro-acetate salt (I-15) was prepared similarly except in step 1 aniline was replaced by 3-chloro-4-methyl-aniline.

(S)-1-Methanesulfonyl-pyrrolidine-2-carboxylic acid {(S)-3-[5-(4,6-dimethyl-pyrimidine-5-carbonyl)-hexahydro-pyrrolo[3,4-c]pyrrol-2-yl]-1-phenyl-propyl}-amide (1-36) was prepared by acylation of 42 with 1-(methylsulfonyl)-(L)-proline mediated by EDCI/HOBT as described in step 1 of example 1 to afford I-36.

### EXAMPLE 3

### 2-Cyclohexyl-N-{(S)-3-[5-(2,6-dimethyl-benzoyl)-hexahydro-pyrrolo[3,4-c]pyrrol-2-yl]-1-phenyl-propyl}-malonamic acid methyl ester (I-20) and 2-cyclohexyl-N-{(S)-3-[5-(2,6-dimethyl-benzoyl)-hexahydro-pyrrolo[3,4-c]pyrrol-2-yl-1-phenyl-propyl}-malonamic acid; trifluoroacetate salt (I-14)

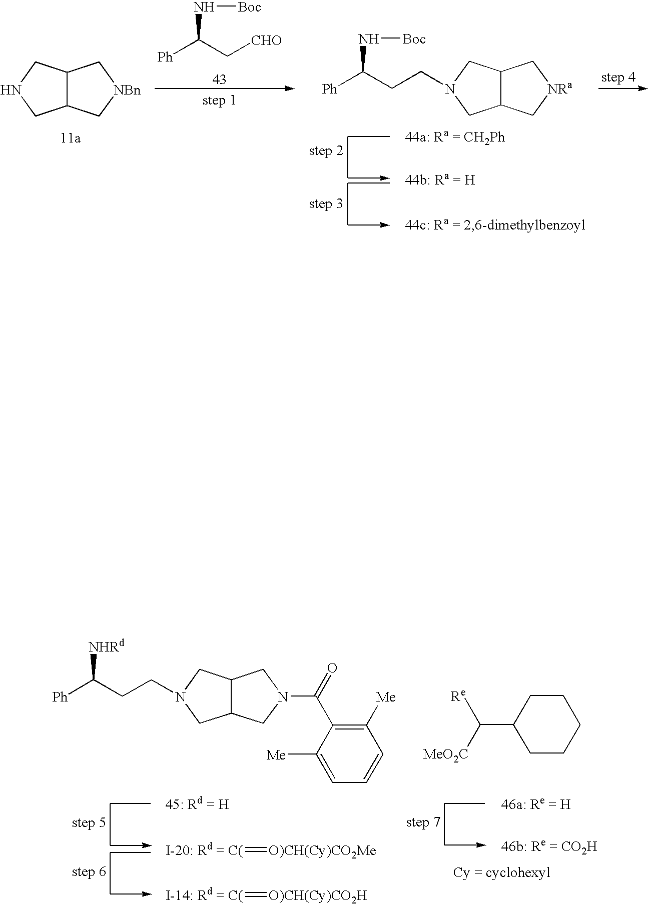 Step 1—A mixture of 43 (4.07 g, 16.3 mmol), 11a (3.0 g, 14.8 mmol), NaBH(OAc)₃ (4.71 g, 22.2 mmol) and HOAc (2.1 mL, 37.1 mmol) in DCM (100 mL) was stirred at RT for 4 h. The reaction was quenched by addition of 5% NaHCO₃. The organic layer was separated and the aqueous layer was extracted with DCM. The combined organic extracts were dried (MgSO₂), filtered and concentrated in vacuo. The residue was purified via SiO₂ chromatography eluting with DCM/MeOH to afford 44a.
Step 2—A mixture of 44a (798 mg, 1.83 mmol), Pd(OH)₂ (catalytic) and ammonium formate (1.16 g) in EtOH (25 mL) was heated at reflux for several hours. It was cooled to RT and the catalyst was filtered off through a CELITE® pad. The filtrate was evaporated and the residue was purified via SiO₂ chromatography eluting with DCM/MeOH/NH₄OH to afford 44b.
Step 3—{(S)-3-[5-(2,6-Dimethyl-benzoyl)-hexahydro-pyrrolo[3,4-c]pyrrol-2-yl]-1-phenyl-propyl}-carbamic acid tert-butyl ester (44c) was prepared from 42b following the procedure described in step 3 of example 2 except 2,6-dimethylbenzoic acid was used in place of cyclopentanecarboxylic acid.
Step 4—A mixture of 44c (1.81 g, 3.77 mmol) and HCl (1.25M in MeOH, 30 mL) was heated at 50° C. for 2 h. The volatiles were evaporated and the residue was purified via SiO₂ chromatography eluting with DCM/MeOH/NH₄OH (60/10/1) to afford 0.896 g (62%) of 45a: M+H=380.
Step 5—A mixture of 45a (97 mg, 0.258 mmol), 2-cyclohexyl-malonic acid monomethyl ester (100 mg, 0.516 mmol), PS-carbodiimide (0.28 g, 0.389 mmol) and HOBt (35 mg, 0.258 mmol) in DCM (5 mL) was stirred at RT overnight. The resin was filtered and washed with DCM, the filtrate was washed with saturated NaHCO₃, dried (Na₂SO₄), filtered and evaporated in vacuo. The residue was purified via SiO₂ chromatography eluting with DCMNMeOH/NH₄OH (60/10/1) to afford 0.105 g of 1-20: M+H=560; MP=83-84° C.; Anal. (C₃₄H₄₅N₃O₄.0.05CH₂Cl₂) C: calcd, 72.51; found, 71.52; H: calcd, 8.06; found, 7.92; N: calcd, 7.45; found, 7.65.
Step 6—A mixture of (±)-I-20 (75 mg, 0.134 mmol) and 2.5M aqueous NaOH in a mixture of THF/MeOH/H₂O (1/1/1, 3 mL) was stirred at RT for 3 h. It was acidified with TFA and concentrated in vacuo. The residue was purified via preparative HPLC to afford I-14: M+H=546.5.
Step 7—To a solution of LDA (1.5M in THF, 8.1 mL, 12.2 mmol) in THF (10 mL) cooled at −78° C. was added dropwise a solution of cyclohexyl-acetic acid methyl ester (46a, 6.09 mmol) in THF (10 mL). After stirring for 2.5 h at −78° C. CO₂ (solid) was added. The reaction was warmed to RT and HCl (2M in water) was added. The mixture was extracted with Et₂O. The combined organic extracts were extracted with saturated NaHCO₃. The aqueous layer was acidified at 0° C. with con HCl and extracted with Et₂O. The organic extract was dried (MgSO₄), filtered and evaporated. The crude 46b was used in step 5 without further purification.

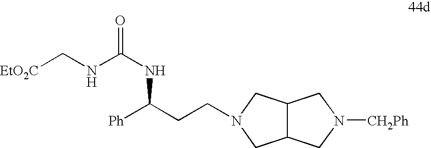

(3-{(S)-3-[5-(2,6-Dimethyl-benzoyl)-hexahydro-pyrrolo[3,4-c]pyrrol-2-yl]-1-phenyl-propyl}-ureido)-acetic acid (I-34) was prepared by removal of the BOC group of 44a according to the procedure in step 4, acylation of resulting amine with 3-isocyanato-acetic acid ethyl ester [CAS Reg. No. 2949-22-6] to afford 44d. The benzyl group was removed by catalytic hydrogenation as described in step 2 of the present example and the amine condensed with 2,6-dimethylbenzoic acid using EDCI as described in step 6 of example 8. Hydrolysis of the ethyl ester with NaOH/THF/H₂O in step 6 of the this example afforded I-34.

4-(3-{(S)-3-[5-(2,6-Dimethyl-benzoyl)-hexahydro-pyrrolo[3,4-c]pyrrol-2-yl]-1-phenyl-propyl}-ureido)-butyric acid (I-35) was prepared analogously except 3-isocyanato-acetic acid ethyl ester was replaced with 4-isocyanato-butyric acid ethyl ester [CAS Reg. No. 106508-62-7].

### EXAMPLE 4

### 2-{(S)-3-[5-(2,6-Dimethyl-benzoyl)-hexahydro-pyrrolo[3,4-c]pyrrol-2-yl]-1-phenyl-propylcarbamoyl}-3-methyl-butyric acid (I-13)

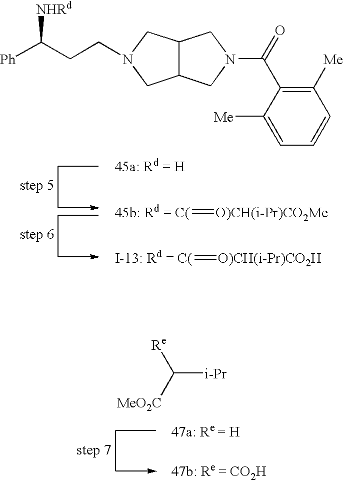

2-Isopropyl-malonic acid monoethyl ester 47b was prepared from 47a following the procedure described for (±)-2-cyclohexyl-malonic acid monomethyl ester (example 3, step 7) except 3-methyl-butyric acid ethyl ester replaced cyclohexyl-acetic acid methyl ester.

2-{(S)-3-[5-(2,6-Dimethyl-benzoyl)-hexahydro-pyrrolo[3,4-c]pyrrol-2-yl]-1-phenyl-propylcarbamoyl}-3-methyl-butyric acid ethyl ester 45b was prepared from 45a following the procedure described in step 5 of example 3 except 46b was replaced with 47b.

2-{(S)-3-[5-(2,6-Dimethyl-benzoyl)-hexahydro-pyrrolo[3,4-c]pyrrol-2-yl]-1-phenyl-propylcarbamoyl}-3-methyl-butyric acid (I-13) was prepared following the procedure described in step 6 of example 3 except I-20 was replaced with 45b.

### EXAMPLE 5

### [4-((3-Chloro-4-methyl-phenyl)-{3-[5-(4,6-dimethyl-pyrimidine-5-carbonyl)-hexahydro-pyrrolo[3,4-c]pyrrol-2-yl]-propyl}-carbamoyl)-2-oxo-pyrrolidin-1-yl]-acetic acid; compound with trifluoro-acetic acid (I-18)

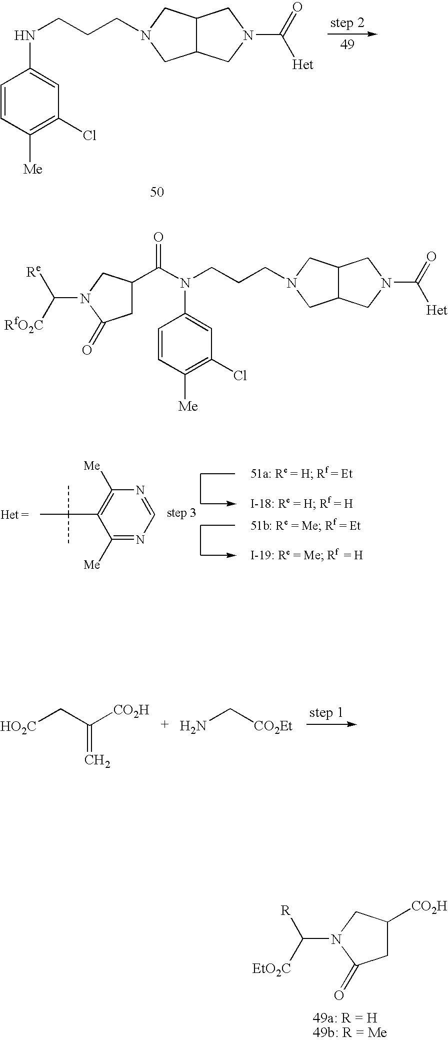 Step 1—A mixture of glycine ethyl ester hydrochloride (1.21 g, 8.69 mmol) and NaOH (347 mg, 8.69 mmol) in water (10 mL) was stirred at RT for 20 min then itaconic acid (48, 1.03 g, 7.90 mmol) was added. The reaction was heated at reflux overnight; cooled to RT and acidified with con HCl. The mixture was extracted with DCM and the organic extract was dried (Na₂SO₄), filtered and evaporated in vacuo to afford 49a which was used without further purifications in the next step.
Step 2—A mixture of 49a (0.16 g, 0.757 mmol), (COCl)₂ (73 μL, 0.824 mmol) and DMF (1 drop) in DCM (5 mL) was stirred at RT for 2 h. The volatiles were then evaporated and the residue was diluted with DCM. To the resulting solution was added 50 (162, 0.379 mmol), followed by TEA ((0.16 mL, 1.14 mmol). The reaction was heated at 40° C. overnight, cooled to RT and washed with saturated NaHCO₃. The organic layer was dried (Na₂SO₄), filtered and evaporated in vacuo. The residue was purified via SiO₂ chromatography eluting with DCM/MeOH/NH₄OH (60/10/1) to afford 0.20 g of 50: [M+H]=625; mp=66.4-68.0° C.
Step 3—[4-((3-Chloro-4-methyl-phenyl)-{3-[5-(4,6-dimethyl-pyrimidine-5-carbonyl)-hexahydro-pyrrolo[3,4-c]pyrrol-2-yl]-propyl}-carbamoyl)-2-oxo-pyrrolidin-1-yl]-acetic acid trifluoroacetate (I-18 TFA salt) was prepared by the procedure described in step 6 of example 3 except 1-20 was replaced with 51a.

2-[4-((3-Chloro-4-methyl-phenyl)-{3-[5-(4,6-dimethyl-pyrimidine-5-carbonyl)-hexahydro-pyrrolo[3,4-c]pyrrol-2-yl]-propyl}-carbamoyl)-2-oxo-pyrrolidin-1-yl]-propionic acid ethyl ester 51b was prepared by the procedure described in step 2 of example 5 except 49a was replaced by 49b. 2-[4-((3-Chloro-4-methyl-phenyl)-{3-[5-(4,6-dimethyl-pyrimidine-5-carbonyl)-hexahydro-pyrrolo[3,4-c]pyrrol-2-y]-propyl}-carbamoyl)-2-oxo-pyrrolidin-1-yl]-propionic acid, trifluoroacetate salt (I-19) was prepared from 51b by the procedure described in step 6 of example 3.

### EXAMPLE 6

### 3-Oxo-cyclopentanecarboxylic acid [(S)-3-[5-(2,4-dimethyl-pyridine-3-carbonyl)-hexahydro-pyrrolo[3,4-c]pyrrol-2-yl]-1-(3-fluoro-phenyl)-propyl]-amide (I-23)

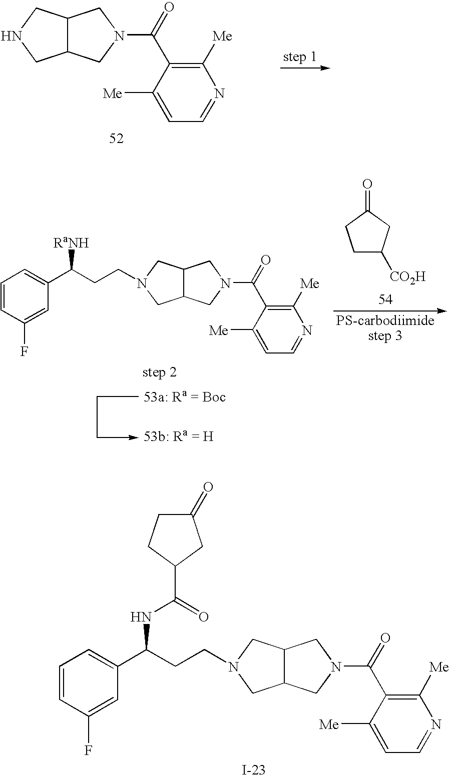

The amine 52 was prepared as described in steps 1 and 2 of example 1 except in step 1,2,4-dimethyl-nicotinic acid was used in place of 4,6-dimethyl-pyrimidine-5-carboxylic acid.

Step 1 was carried out as described in step 2 of example 3 except 40b was replaced with 52b to afford 52a. Step 2 was carried out as described in step 4 of example 3 to afford 53b.

Step 3—A mixture of 53b (138 mg, 0.34 mmol), 3-oxo-cyclopentanecarboxylic acid (55 mg, 0.38 mmol) and PS-carbodiimide (PS=polymer supported; loading 1.35 mmol/g, 514 mg, 0.69 mmol) in DCM (5 mL) was stirred overnight at RT. The resin was filtered off and washed with DCM (5 mL). The filtrate was evaporated onto SiO₂ and it was purified via SiO₂ chromatography eluting with DCM/MeOH to afford 0.139 g (77%) of I-23 as white foam: Anal: (C₂₉H₃₅FN₄O₃.0.50 mol DCM) Calcd: C: 64.43; H: 6.37; N: 10.43. Found: C: 64.53; H: 6.61; N: 10.20.

3-Oxo-cyclohexanecarboxylic acid [(S)-3-[5-(4,6-dimethyl-pyrimidine-5-carbonyl)-hexahydro-pyrrolo[3,4-c]pyrrol-2-yl]-1-(3-fluoro-phenyl)-propyl]-amide (I-24) was prepared from 53b as described in step 3 (supra) except 3-oxo-cyclopentanecarboxylic acid was replaced with 3-oxo-cyclohexanecarboxylic acid: Anal: (C₂₉H₃₆FN₅O₃.0.35 mol DCM)Calcd: C: 63.80; H: 6.47; N: 12.82. Found: C: 63.94; H: 6.71; N: 12.70.

### EXAMPLE 7

### 2-Oxo-cyclopentanecarboxylic acid [(S)-3-[5-(4,6-dimethyl-pyrimidine-5-carbonyl)-hexahydro-pyrrolo[3,4-c]pyrrol-2-yl]-1-(3-fluoro-phenyl)-propyl]-amide (I-17)

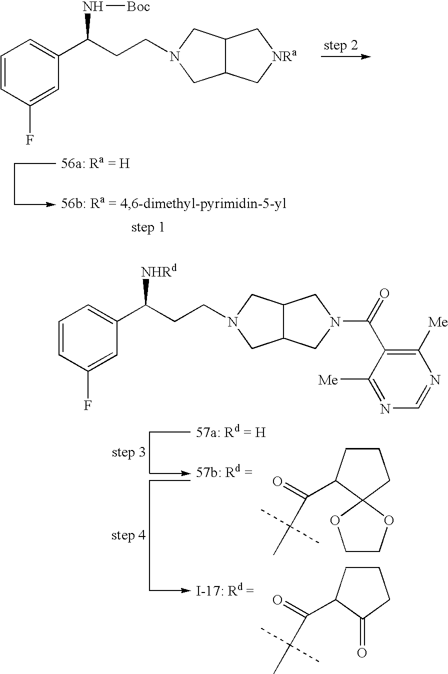 Step 1—Acylation of 56a with 4,6-dimethyl-pyrimidine-5-carboxylic acid was carried out with TBTU by the following general procedure.
General procedure for O-(Benzotriazol-1-yl)-N,N,N′,N′-tetramethyluronium tetrafluoroborate coupling—The carboxylic acid (1.1 equivalent) and 56a (1.0 equivalent) are dissolved in DCM and TEA (4 equivalents) is added TBTU (1.2 equivalents) and the resulting solution is stirred at RT until the reaction is complete. The resulting mixture is partitioned between EtOAc and H₂O. The combined organic extracts are combined, washed with H₂O and brine and evaporated.
Step 2—Removal of the BOC protecting group to afford 51a was carried out as described in step 4 of example 3.
Step 3—Acylation of 51a with 1,4-dioxa-spiro[4.4]nonane-6-carboxylic acid (S. Gregory et al., Bioorg. Med. Chem. 2002 10(12):4143-4154) to afford 51b was carried out by the procedure described in step 1 of this example.
Step 4—A solution of 51b (130 mg, 0.25 mmol), 1M aqueous HCl (4 mL) and THF (4 mL) was stirred at RT for 8 h and at 4° C. for an additional 62 h. The reaction was made basic with saturated NaHCO₃ and thrice extracted with EtOAc (25 mL). The combined extracts were dried (MgSO₄), filtered and evaporated onto SiO₂. The residue was purified via SiO₂ chromatography eluting with DCM/MeOH to afford 0.089 g (75%) of I-17 as a white foam: (C₂₈H₃₄FN₅O₃.0.35 mol DCM) Calcd C: 63.37; H: 6.51; N: 13.03 Found: C: 63.31; H: 6.57; N: 13.02.

### EXAMPLE 8

### 3,3-Difluoro-cyclobutanecarboxylic acid {(S)-1-(3-cyano-phenyl)-3-[5-(4,6-dimethyl-pyrimidine-5-carbonyl)-hexahydro-pyrrolo[3,4-c]pyrrol-2-yl]-propyl}-amide (I-21)

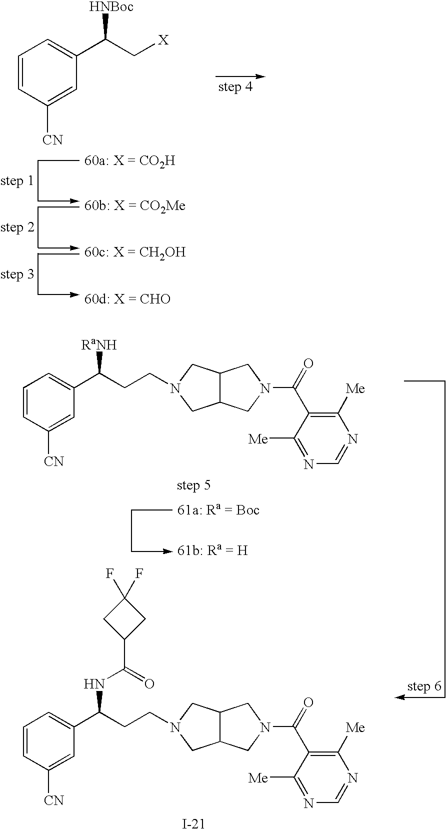 Step 1—DCC (853 mg, 4.1 mmol) and DMAP (42 mg, 0.34 mmol) were added to an ice-cold solution of (S)-3-tert-butoxycarbonylamino-3-(3-cyano-phenyl)-propionic acid (60a; 1.00 g, 3.44 mmol) in MeOH (10 mL). The mixture was stirred overnight at RT. The reaction mixture was filtered and the filter cake was washed with a small amount of MeOH. The filtrate was evaporated onto SiO₂ and purified via SiO₂ chromatography eluting with hexane/EtOAc) to afford 1.20 g of 60b.
Step 2—LiBH₄ (111 mg, 5.09 mmol) was added to a ice-cold solution of 60b (516 mg, 1.7 mmol) in Et₂O (30 mL) maintained under an N₂ atmosphere. The reaction was stirred at 0° C. for 45 min and quenched by addition of saturated NH₄Cl (10 mL). The mixture was stirred vigorously for 20 minutes at RT. The aqueous layer was separated and extracted twice with Et₂O (25 mL). The combined organic extracts were washed with brine, dried (MgSO₄), filtered and evaporated. The crude residue was purified via SiO₂ chromatography eluting with hexane/EtOAc to afford 0.382 g (78%) of 60c.
Step 3—A solution of 60c (450 mg, 1.63 mmol) and TEA (0.69 mL, 4.96 mmol) in a mixture of DCM/DMSO (1/1, 4.5 mL) was added dropwise to an ice-cold solution of SO₃.pyridine complex (790 mg, 4.96 mmol) and 1:1 DCM/DMSO (9 mL). The reaction was stirred at 0° C. for 3 h then diluted with water (20 mL) and warmed to RT. The mixture was extracted twice with toluene (25 mL) and the combined organic extracts were washed twice with 0.5 M HCl (50 mL) and brine, dried (MgSO₄), filtered and evaporated. The crude residue was purified via SiO₂ chromatography eluting with hexane/EtOAc to afford 60d as a viscous liquid that crystallized upon standing.
Step 4—A mixture of 60d (235 mg, 0.85 mmol), (4,6-dimethyl-pyrimidin-5-yl)-hexahydro-pyrrolo[3,4-c]pyrrol-2-yl-methanone (40b, 211 mg, 0.85 mmol), Na(OAc)₃BH (236 mg, 1.11 mmol) and HOAc (0.13 mL, 2.22 mmol) in DCM (9 mL) was stirred at room temperature overnight. The reaction was quenched by addition of 10% aqueous K₂CO₃ (10 mL) and stirred for 30 min. The organic layer was separated and the aqueous was extracted with DCM (15 mL). The combined organic extracts were washed brine, dried (Na₂SO₄), filtered and evaporated. The crude residue was purified via SiO₂ chromatography eluting with DCM/MeOH to afford 0.288 g (67%) of 61a as a white foam.
Step 5—To a mixture of 61a (288 mg, 0.57 mmol) in HCl (4 M in 1,4-dioxane, 10 mL) was added HCl (10 M in MeOH) to produce a homogeneous solution. The reaction was stirred at RT for 1 h and the solvent was evaporated under a stream of N₂. The residue was stirred with 20% aqueous K₂CO₃ (10 mL) and extracted twice with DCM (20 mL). The combined extracts were dried (Na₂SO₄), filtered and evaporated. The crude amine 61b was a yellow viscous liquid which was used in the next step.
Step 6—A mixture of 61b (75 mg, 0.185 mmol), 3,3-difluoro-cyclobutanecarboxylic acid (30 mg), EDCI (46.2 mg, 0.241 mmol), HOBt.H₂O (32.6 mg, 0.241 mmol) and DIPEA (97 μL, 0.556 mmol) in DCM (3 mL) was stirred overnight. The mixture was evaporated onto SiO₂ and the residue was purified via SiO₂ chromatography to afford I-21: M+H=523; Anal. C₂₈H₃₂F₂N_{6O}₂.0.30 mol DCM) Calcd: C: 62.02; H: 6.00; N: 15.33; Found: C: 62.02; H; 5.77; N: 15.36.

4,4-Difluoro-cyclohexanecarboxylic acid {(S)-1-(3-cyano-phenyl)-3-[5-(4,6-dimethyl-pyrimidine-5-carbonyl)-hexahydro-pyrrolo[3,4-c]pyrrol-2-yl]-propyl}-amide (I-22) was made analogously except in step 6, 4,4-difluoro-cyclohexanecarboxylic acid was used in place of 3,3-difluorocyclobutane-carboxylic acid: Anal. (C₃₀H₃₆F₂N₆O₂.1.0 mol DCM) Calcd: C: 62.04; H: 6.38; N: 14.00; Found: C: 61.56; H: 6.06; N: 14.20.

### EXAMPLE 9

### 3-(3-(3-Chloro-4-methyl-phenyl)-3-{3-[5-(4,6-dimethyl-pyrimidine-5-carbonyl)-hexahydro-pyrrolo[3,4-c]pyrrol-2-yl]-propyl}-ureido)-propionic acid, trifluoroacetate salt (I-8)

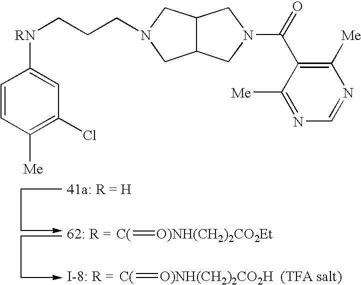

A mixture of 41a (30 mg, 0.07 mmol) and 3-isocyanato-propionic acid ethyl ester (17 mg, 0.105 mmol) in DCM (2 mL) was stirred at RT for 18 h. The reaction mixture was filtered and the filter cake was washed with DCM. The filtrate was evaporated and half of the residue was purified by preparative HPLC to afford 62. The other half was dissolved in MeOH (1 mL) and 10% aqueous NaOH (0.6 mL). The reaction was stirred at RT for 18 h and the reaction was adjusted to pH of 1 with TFA. The resulting solution was evaporated mixture was evaporated and the residue was suspended in DCM/MeOH (9/1), sonicated for 10 minutes and filtered through a ChemElut (1 mL unbuffered) cartridge. The filtrate was evaporated and purified via SiO₂ HPLC to afford 1-8.

4-(3-(3-Chloro-4-methyl-phenyl)-3-{3-[5-(4,6-dimethyl-pyrimidine-5-carbonyl)-hexahydro-pyrrolo[3,4-c]pyrrol-2-yl]-propyl}-ureido)-butyric acid, trifluoro-acetate salt (I-1) was prepared analogously except 3-isocyanato-propionic acid ethyl ester was replaced with 4-isocyanato-butyric acid ethyl ester.

### EXAMPLE 10

### [4-((3-Chloro-4-methyl-phenyl)-{3-[5-(2,4-dimethyl-pyridine-3-carbonyl)-hexahydro-pyrrolo[3,4-c]pyrrol-2-yl]-propyl}-carbamoyl)-2-oxo-piperidin-1-yl]-acetic acid trifluoroacetate (I-27)

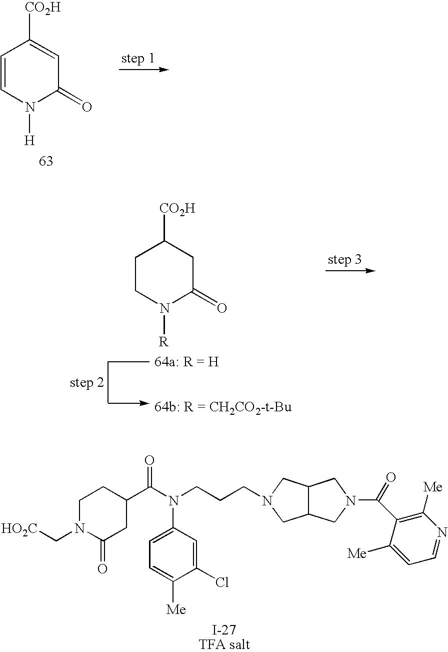

(2,4-Dimethyl-pyridin-3-yl)-(hexahydro-pyrrolo[3,4-c]pyrrol-2-yl)-methanone (65) was prepared by acylation of 11a as described in step 1 of example 1 except 4,6-dimethyl-pyrimidine-5-carboxylic acid was replaced by 2,4-dimethyl-pyridine-3-carboxylic acid. {(5-[3-(3-Chloro-4-methyl-phenylamino)-propyl]-hexahydro-pyrrolo[3,4-c]pyrrol-2-yl}-(2,4-dimethyl-pyridin-3-yl)-methanone (66) was prepared by alkylation of 65 with (3-chloro-4-methyl-phenyl)-(3-chloro-propyl)-amine as described in step 3 of example 1. (3-Chloro-4-methyl-phenyl)-(3-chloro-propyl)-amine was prepared by alkylation of 2-chloro-4-amino-toluene with 1-chloro-3-iodo-propane as described in step 1 of example 2.

Step 1—A mixture of 2-oxo-1,2-dihydro-pyridine-4-carboxylic acid (63; 0.5 g, 3.59 mmol) and 20% Pd(OH)_{2/C} (0.2 g) in MeOH (50 mL) was hydrogenated in a Parr apparatus for 48 h at 55 PSI of H₂. The catalyst was filtered off and the filtrate was evaporated to afford 64a which was used without further purification.

Step 2—To a solution cooled solution (0-5° C.) of 64a (0.25 g, 1.74 mmol) in DMF (3 mL) was added NaH (60% in mineral oil, 0.153 g, 3.84 mmol). The suspension was stirred at 0-5° C. for 10 min and then warmed to RT and stirred for 18 h. The volatiles were evaporated, water was added, the mixture was washed with Et₂O and the aqueous was acidified (pH 1) by addition of 10% aqueous HCl. The mixture was extracted 3 times with DCM (50 mL), the combined organics were dried (MgSO₄), filtered and evaporated to afford 0.124 g) of 64b which was used in the next step without further purification.

Step 3—Oxalyl chloride (0.04 mL, 0.46 mmol) was added dropwise to a mixture of 64b (100 mg, 0.38 mmol) and pyridine (0.08 mL, 0.99 mmol) in DCM (3 mL) maintained under N₂ atmosphere at RT. The reaction was stirred for 25 min then a solution of 66 (149 mg, 0.34 mmol), DIPEA (0.237 mL, 1.36 mmol) and DMAP (5 mg, 0.038 mmol) in DCM was added dropwise. The mixture was stirred for 18 h at RT, concentrated and the residue was dissolved in a mixture 10% TFA in DCM (1/9, 5 mL) and stirred at RT for 18 h. The volatiles were evaporated and the residue was purified via SiO₂ chromatography eluting with DCM/MeOH/HOAc to afford I-27 that was stripped with MeOH and toluene, and then triturated with cyclohexane provide a hygroscopic foam.

### EXAMPLE 11

### (1R,2S)-Cyclopentane-1,2-dicarboxylic acid 1-dimethylamide 2-({(S)-3-[5-(4,6-dimethyl-pyrimidine-5-carbonyl)-hexahydro-pyrrolo[3,4-c]pyrrol-2-yl]-1-phenyl-propyl}-amide)trifluoroacetate (I-37)

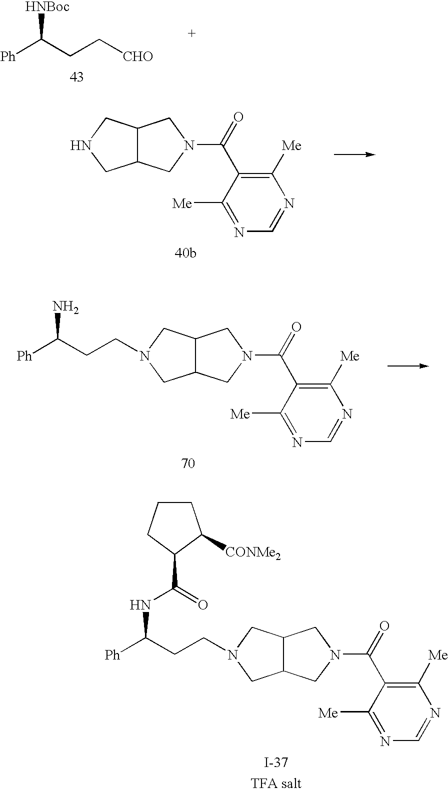

{(S)-3-[5-(4,6-Dimethyl-pyrimidine-5-carbonyl)-hexahydro-pyrrolo[3,4-c]pyrrol-2-yl]-1-phenyl-propyl}-carbamic acid tert-butyl ester (70) was prepared in the same manner as 61b in example 8 except ((S)-3-oxo-1-phenyl-propyl)-carbamic acid tert-butyl ester (CAS Reg No. 135865-78-0) was used in place of [(S)-1-(3-cyano-phenyl)-3-oxo-propyl]-carbamic acid tert-butyl ester. Removal of the BOC protecting group was accomplished with HCl as described in step 4 of example 3.

A mixture of 70 (27 mg, 0.071 mmol) and cis-1,2-cyclopentanedicarboxylic anhydride (10.5 mg, 0.074 mmol) in DCM (1 mL) was stirred at RT for 18 h. The volatiles were evaporated and the residue was treated with EEDQ (20 mg, 0.078 mmol) and Me₂NH (2M in THF, 2 mL). The reaction was stirred at RT for 18 h and heated at 60° C. for 24 h. The reaction mixture was cooled, evaporated and purified by preparative HPLC to afford I-37.

2-{(S)-3-[5-(2,6-Dimethyl-benzoyl)-hexahydro-pyrrolo[3,4-c]pyrrol-2-yl]-1-phenyl-propylcarbamoyl}-cyclopentanecarboxylic acid (I-33) was prepared analogously except 70 was replaced with 45a (example 3) and the Me₂NH amidation step was omitted.

### EXAMPLE 12

### [4-((3-Chloro-4-methyl-phenyl)-{3-[5-(4,6-dimethyl-pyrimidine-5-carbonyl)-hexahydro-pyrrolo[3,4-c]pyrrol-2-yl]-propyl}-carbamoyl)-piperidin-1-yl]-acetic acid tert-butyl ester (I-25) and [4-((3-Chloro-4-methyl-phenyl)-{3-[5-(4,6-dimethyl-pyrimidine-5-carbonyl)-hexahydro-pyrrolo[3,4-c]pyrrol-2-yl]-propyl}-carbamoyl)-piperidin-1-yl]-acetic acid, trifluoroacetate salt (I-3)

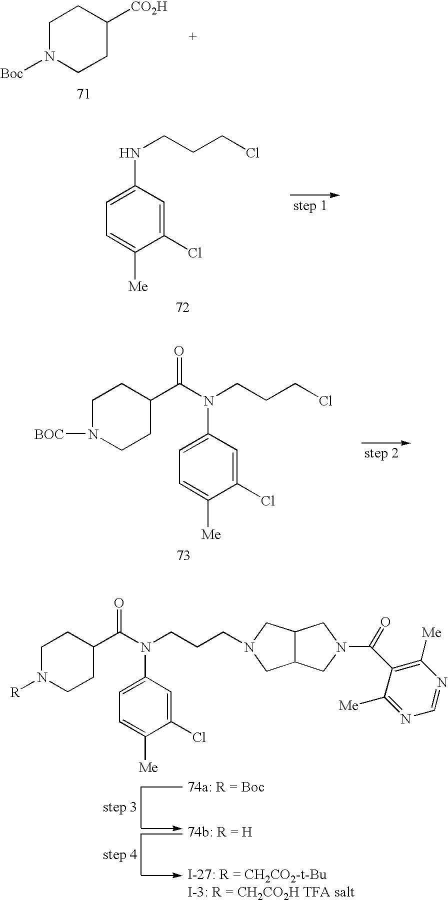 Step 1—Oxalyl chloride (1.17 mL, 13.5 mmol) was added dropwise to a solution 71 (2.83 g, 12.4 mmol) and pyridine (2.31 mL, 28.6 mmol) in DCM (15 mL) maintained under N₂ atmosphere at RT. The reaction was stirred for 25 min then a solution of 72 (2.45 g, 11.2 mmol), DIPEA (6.85 mL, 39.3 mmol) and DMAP (0.137 mg, 1.12 mmol) in DCM was added dropwise. The reaction was stirred for 18 h, quenched by addition of saturated NaHCO₃ and extracted with DCM. The combined organic extracts were dried (MgSO₄), filtered and evaporated. The residue was purified via SiO₂ chromatography to afford 2.45 g (51%) of 73.
Step 2—A mixture of 73 (1.24 g, 2.90 mmol), 40b (0.65 g, 2.63 mmol), KI (0.48 g, 2.90 mmol) and DIPEA (0.92 mL, 5.27 mmol) in acetonitrile was heated to 140° C. for 3 h in a laboratory microwave. The reaction was quenched by addition of saturated NaHCO₃ and extracted 3 times with DCM (50 mL). The combined organic extracts were dried (MgSO₄), filtered and evaporated. The residue was purified via SiO₂ chromatography eluting with DCM/MeOH/NH₄OH to afford 1.01 g (60%) of 74a.
Step 3—A mixture of 74a (1.01 g, 1.58 mmol) in 1:9 TFA/DCM (10 mL) was stirred at RT for 18 h. The volatiles were evaporated and the residue was co-evaporated with toluene and purified via SiO₂ chromatography eluting with DCM/MeOH/NH₄OH to afford 0.69 g (81%) of 74b.
Step 4—Bromo-acetic acid tert-butyl ester (11.5 μL, 0.077 mmol) was added to a solution of 74b (38 mg, 0.07 mmol), DIPEA (49 μL, 0.28 mmol) in DCE (2 mL). The reaction was stirred at RT for 48 h, then heated to 40° C. for 24 h. A second aliquot of 11.5 μL of bromo-acetic acid tert-butyl ester was then added and stirring continued at 40° C. for 24 hours. Excess KI was added and the reaction was stirred at 60° C. for 24 h then quenched with water (0.7 mL). The mixture was filtered through a ChemElut (1 mL unbuffered) cartridge. The filtrate was evaporated and half of it was purified via preparative HPLC to afford I-27 and the remaining was stirred for 3 h with DCM/TFA/Et₃SiH. The reaction mixture was evaporated and co-evaporated with DCM, the residue was purified via preparative HPLC to afford I-3.

3-[4-((3-Chloro-4-methyl-phenyl)-{3-[5-(4,6-dimethyl-pyrimidine-5-carbonyl)-hexahydro-pyrrolo[3,4-c]pyrrol-2-yl]-propyl}-carbamoyl)-piperidin-1-yl]-propionic acid tert-butyl ester, trifluoro-acetate salt (I-10) and 3-[4-((3-chloro-4-methyl-phenyl)-{3-[(5-(4,6-dimethyl-pyrimidine-5-carbonyl)-hexahydro-pyrrolo[3,4-c]pyrrol-2-yl]-propyl}-carbamoyl)-piperidin-1-yl]-propionic acid; trifluoro-acetate salt (I-12) were prepared in analogous manner except in step 4, bromo-acetic acid tert-butyl ester was replaced with 3-bromo-propionic acid tert-butyl ester.

4-[4-((3-Chloro-4-methyl-phenyl)-{3-[5-(4,6-dimethyl-pyrimidine-5-carbonyl)-hexahydro-pyrrolo[3,4-c]pyrrol-2-yl]-propyl}-carbamoyl)-piperidin-1-yl]-butyric acid tert-butyl ester, trifluoro-acetate salt (I-11) and 4-[4-((3-chloro-4-methyl-phenyl)-{3-[5-(4,6-dimethyl-pyrimidine-5-carbonyl)-hexahydro-pyrrolo[3,4-c]pyrrol-2-yl]-propyl}-carbamoyl)-piperidin-1-yl]-butyric acid, trifluoro-acetate salt (I-5) were prepared in analogous manner except in step 4, bromo-acetic acid tert-butyl ester was replaced with 4-bromo-butyric acid tert-butyl ester.

2-[4-((3-Chloro-4-methyl-phenyl)-{3-[5-(4,6-dimethyl-pyrimidine-5-carbonyl)-hexahydro-pyrrolo[3,4-c]pyrrol-2-yl]-propyl}-carbamoyl)-piperidin-1-yl]-2-oxo-ethoxy}-acetic acid; trifluoro-acetate salt (I-6) was prepared by acylation of 74b with 1,4-dioxane-2,6-dione [CAS Reg No. 4480-83-5].

### EXAMPLE 13

### 2-[4-((3-Chloro-4-methyl-phenyl)-{3-[5-(4,6-dimethyl-pyrimidine-5-carbonyl)-hexahydro-pyrrolo[3,4-c]pyrrol-2-yl]-propyl}-carbamoyl)-piperidin-1-yl]-3-methyl-but-2-enoic acid ethyl ester (I-2)

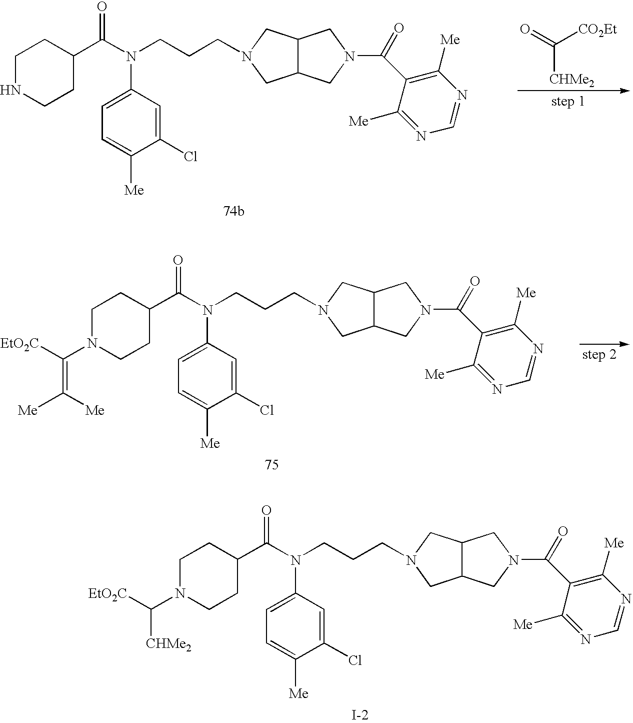 Steps 1—A mixture of 74b (162 mg, 0.3 mmol) and ethyl-3-methyl-2-oxobutyrate (0.135 mL, 0.9 mmol) in DCE (3 mL) was stirred overnight. The reaction mixture was cooled to 0° C. and NaBH(OAc)₃ (129 mg, 0.6 mmol) was then added in 5 portions over 30 min period. The reaction was allowed to stir at RT for 48 h, and additional aliquot of NaBH(OAc)₃ (129 mg) was added and stirring continued for an additional 48 h. The reaction was quenched with saturated NaHCO₃ and extracted 4 times with DCM (30 mL). The combined organic extracts were dried (MgSO₄), filtered and evaporated. The residue was purified via SiO₂ chromatography eluting with DCM/MeOH/NH₄OH to afford 0.058 g of 75 containing 8% of an impurity.
Step 2—The product from step 1 was dissolved in DCM/TFA/Et₃SiH (7/2/1, 2 mL) and stirred at RT for 6 days. The volatiles were evaporated and the residue was purified via preparative HPLC to afford 1-2.

### EXAMPLE 14

### 3-(3-(3-Chloro-4-methyl-phenyl)-3-{3-[5-(4,6-dimethyl-pyrimidine-5-carbonyl)-hexahydro-pyrrolo[3,4-c]pyrrol-2-yl]-propyl}-ureido)-benzenesulfonamide (I-28)

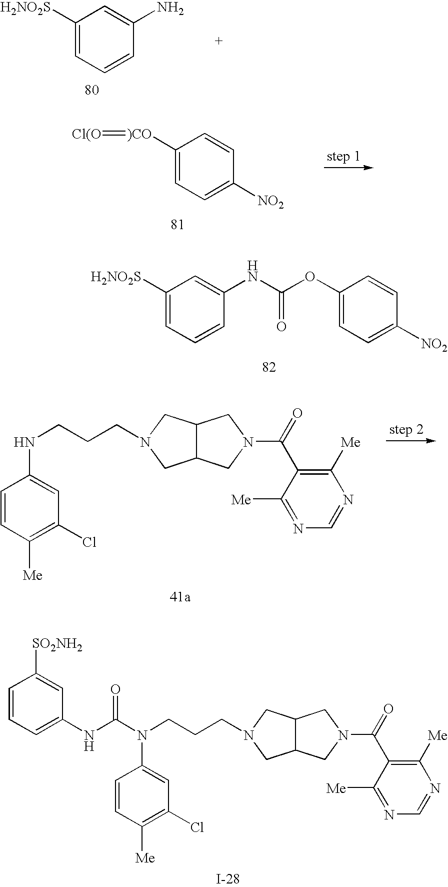 Step 1—To a suspension of 3-amino-benzenesulfonamide (80, 100 mg, 0.581) in dry MeCN (3 mL) maintained under a N₂ atmosphere was added NaHCO₃ (98 g, 1.16 mmol) followed by 4-nitrophenyl chloroformate (81, 117 mg, 0.58 mmol). THF (1 mL) was added to facilitate the dissolution of the amine. The mixture was stirred for 1.5 h and the resulting solution containing 82 was used as is for the next reaction.
Step 2—An aliquot of the above obtained solution (1.61 mL, 0.234 mmol) was added to a solution of 41a (100 mg, 0.234 mmol) in THF (3 mL) followed by TEA (65 μL, 0.468 mmol). The reaction was stirred at RT for 1.5 h, evaporated and the residue was partitioned between EtOAc and water. The organic layer was separated and the aqueous was extracted twice with EtOAc. The combined organics extracts were washed several times with saturated NaHCO₃, dried (MgSO₄), filtered and evaporated. The residue was redissolved in DCM and washed with saturated NaHCO₃ re-dried (MgSO₄), filtered and evaporated to afford an off-white foam which was purified by SiO₂ chromatography eluting with DCM/MeOH to afford 0.045 g (31%) of I-28.

### EXAMPLE 15

### (1S,2S)-2-((3-Chloro-4-methyl-phenyl)-{3-[5-(2,6-dimethyl-benzoyl)-hexahydro-pyrrolo[3,4-c]pyrrol-2-yl]-propyl}-carbamoyl)-cyclohexanecarboxylic acid; trifluoro-acetate salt (I-29)

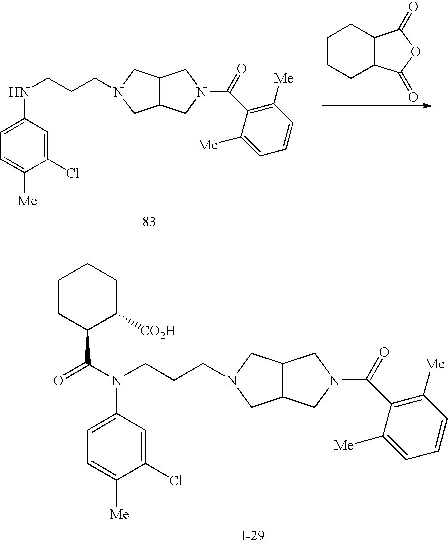

The amine 83 was prepared as was described for 41a (example, steps 1-3) except in step 1, 4,6-dimethyl-pyrimidine-5-carboxylic acid was replaced by 2,6-dimethylbenzoic acid. A mixture of 83 (27 mg, 0.071 mmol) and trans-1,2-cyclohexanedicarboxylic anhydride (10.5 mg, 0.074 mmol) in DCM (1 mL) was stirred at RT for 18 h. The volatiles were evaporated and the residue purified to afford I-29.

(1R,3S)-3-((3-Chloro-4-methyl-phenyl)-{3-[5-(2,6-dimethyl-benzoyl)-hexahydro-pyrrolo[3,4-c]pyrrol-2-yl]-propyl}-carbamoyl)-cyclohexanecarboxylic acid, trifluoro-acetate salt (I-30) was prepared analogously except trans-1,2-cyclohexanedicarboxylic anhydride was replaced with 3-oxabicyclo[3.3.1]nonane-2,4-dione [CAS Reg No.4355-31-1].

(1S,3R)-3-((3-Chloro-4-methyl-phenyl)-{3-[5-(2,6-dimethyl-benzoyl)-hexahydro-pyrrolo[3,4-c]pyrrol-2-yl]-propyl}-carbamoyl)-cyclopentanecarboxylic acid, trifluoro-acetate salt (I-4) was prepared analogously except trans-1,2-cyclohexanedicarboxylic anhydride was replaced with 3-oxabicyclo[3.2.1]octane-2,4-dione [CAS Reg No.6504-16-6].

(1R,2S)-2-((3-Chloro-4-methyl-phenyl)-{3-[5-(4,6-dimethyl-pyrimidine-5-carbonyl)-hexahydro-pyrrolo[3,4-c]pyrrol-2-yl]-propyl}-carbamoyl)-cyclopentanecarboxylic acid; trifluoro-acetate salt (I-7) was prepared analogously except trans-1,2-cyclohexanedicarboxylic anhydride was replaced with cis-1,2-cyclopentanedicarboxylic and 83 was replaced with {5-[3-(4-chloro-3-methyl-phenylamino)-propyl]-hexahydro-pyrrolo[3,4-c]pyrrol-2-yl}-(4,6-dimethyl-pyrimidin-5-yl)-methanone (41a)

### EXAMPLE 16

### 4-((3-Chloro-4-methyl-phenyl)-{3-[5-(4,6-dimethyl-pyrimidine-5-carbonyl)-hexahydro-pyrrolo[3,4-c]pyrrol-2-yl]-propyl}-carbamoyl)-cyclohexanecarboxylic acid, trifluoro-acetate salt (I-32)

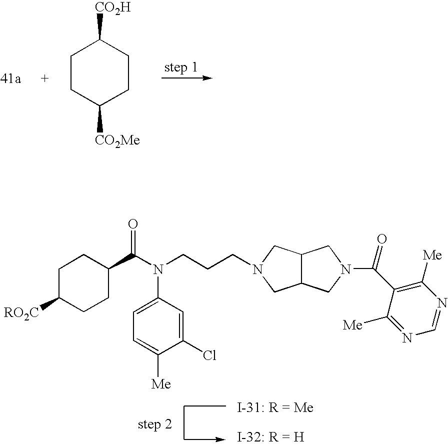

cis-Cyclohexane-1,4-dicarboxylic acid monomethyl ester [CAS Reg. No. 1011-85-4] was converted into the corresponding acid chloride with oxalyl chloride and condensed with 41a using the procedure described in step 2 of example 5 to afford I-31. Hydrolysis of the ester as described in step 6 of example 17 afforded I-32.

4-((3-Chloro-4-methyl-phenyl)-{3-[5-(4,6-dimethyl-pyrimidine-5-carbonyl)-hexahydro-pyrrolo[3,4-c]pyrrol-2-yl]-propyl}-carbamoyl)-cyclohexanecarboxylic acid, trifluoro-acetate salt (I-26) was prepared analogously except cis-cyclohexane-1,4-dicarboxylic acid monomethyl ester was replaced with trans-cyclohexane-1,4-dicarboxylic acid monomethyl ester [CAS Reg. No. 15177-67-0]

### EXAMPLE 17

### 3-Oxo-cyclohexanecarboxylic acid {(S)-1-phenyl-3-[5-(1,2,4-trimethyl-6-oxo-1,6-dihydro-pyridine-3-carbonyl)-hexahydro-pyrrolo[3,4-c]pyrrol-2-yl]-propyl}-amide (II-8)

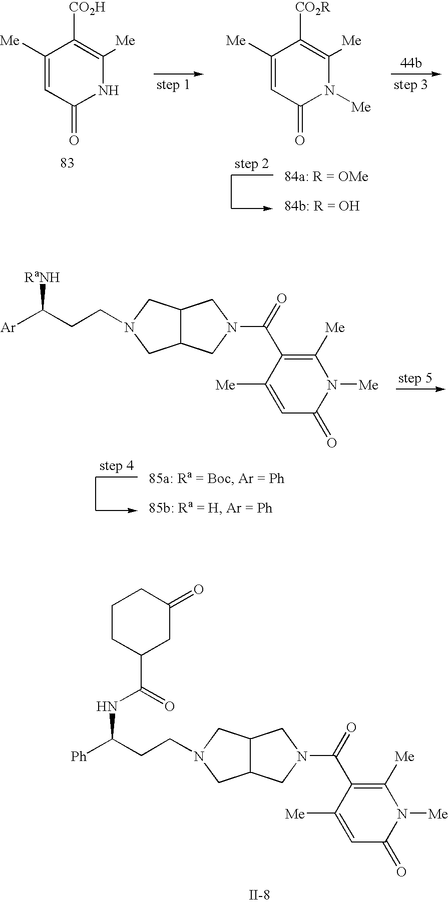 Step 1—A mixture of 2,4-dimethyl-6-oxo-1,6-dihydro-pyridine-3-carboxylic acid (83, 560 mg, 3 mmol), MeI (1.28 g, 9 mmol) and Cs₂CO₃ (3.26 g, 10 mmol) in MeCN was stirred at RT overnight. The reaction was poured into water and extracted 3 times with EtOAc. The combined organic extracts were dried (Na₂SO₄), filtered and evaporated. The crude residue was purified via SiO₂ chromatography eluting with DCM/MeOH/NH₄OH to afford 0.460 g of 84a.
Step 2—1,2,4-Trimethyl-6-oxo-1,6-dihydro-pyridine-3-carboxylic acid methyl ester (84a) was hydrolyzed to afford 84b following the procedure described in step 6 of example 3.
Step 3—{(S)-1-Phenyl-3-[5-(1,2,4-trimethyl-6-oxo-1 ,6-dihydro-pyridine-3-carbonyl)-hexahydro-pyrrolo[3,4-c]pyrrol-2-yl]-propyl}-carbamic acid tert-butyl ester (85a) was prepared by acylation of 44b with 84b with TBTU as described in step 1 of example 7.
Step 4—To a stirred solution of 85a (265 mg, 0.5 mmol) in DCM was added dropwise HCl (4M in 1,4-dioxane, 0.5 mL) dropwise. The reaction was stirred at RT for 90 minutes; the solvent was removed in vacuo and the residue was stripped with DCM. The crude amine 85b was used for next step without further purifications.
Step 5—A mixture of 85b (138 mg, 0.34 mmol), 3-oxo-cyclohexanecarboxylic acid (0.38 mmol) and PS-carbodiimide (loading 1.35 mmol/g, 514 mg, 0.69 mmol) in DCM (5 mL) is stirred overnight at RT. The resin is filtered off and washed with DCM (5 mL). The filtrate is evaporated onto SiO₂ and it purified via SiO₂ chromatography to afford II-8.

3-Oxo-cyclopentanecarboxylic acid {(S)-1-(3-fluoro-phenyl)-3-[5-(1,2,4-trimethyl-6-oxo-1,6-dihydro-pyridine-3-carbonyl)-hexahydro-pyrrolo[3,4-c]pyrrol-2-yl]-propyl}-amide (II-7) was prepared analogously except in step 3, 44b was replaced with [(S)-1-(3-fluoro-phenyl)-3-(hexahydro-pyrrolo[3,4-c]pyrrol-2-yl)-propyl]-carbamic acid tert-butyl ester and in step 5, 3-oxo-cyclohexanecarboxylic acid was replaced with 3-oxo-cyclopentanecarboxylic acid.

### EXAMPLE 18

### 3-Oxo-cyclopentanecarboxylic acid {(S)-1-(3-chloro-phenyl)-3-[5-(1,4,6-trimethyl-2-oxo-1,2-dihydro-pyrimidine-5-carbonyl)-hexahydro-pyrrolo[3,4-c]pyrrol-2-yl]-propyl}-amide (II-4)

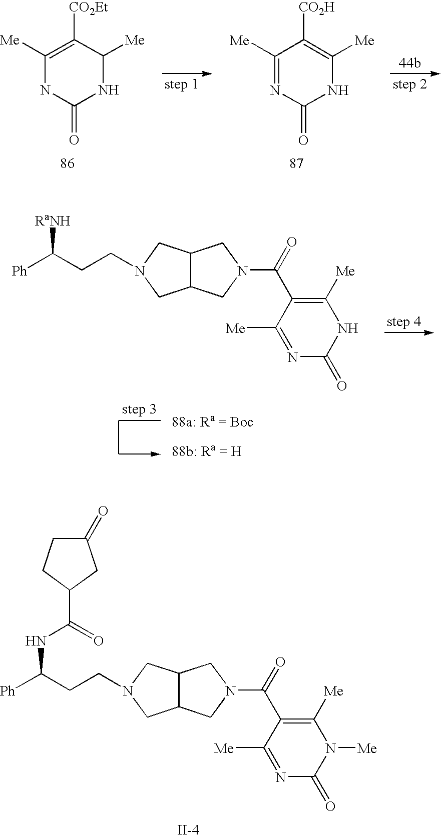

4,6-Dimethyl-2-oxo-1,2,3,4-tetrahydro-pyrimidine-5-carboxylic acid ethyl ester (86) was prepared as described in Tetrahedron 2002 58:4801-4807. 4,6-Dimethyl-2-oxo-1,2-dihydro-pyrimidine-5-carboxylic acid (87) was prepared from 86 as described in J. Heterocyclic Chem. 2001 38:1345.

4,6-Dimethyl-2-oxo-1,2-dihydro-pyrimidine-5-carboxylic acid (87) is converted to II-4 by the procedure described in steps 3-5 of example 17 except in the final step 3-oxo-cyclohexanecarboxylic acid is replaced with 3-oxo-cyclopentanecarboxylic acid.

### EXAMPLE 19

### 3-Oxo-cyclopentanecarboxylic acid (3-{5-[2-(1-carbamoyl-1-methyl-ethoxy)-4,6-dimethyl-pyrimidine-5-carbonyl]-hexahydro-pyrrolo[3,4-c]pyrrol-2-yl}-1-phenyl-propyl)-amide (II-6)

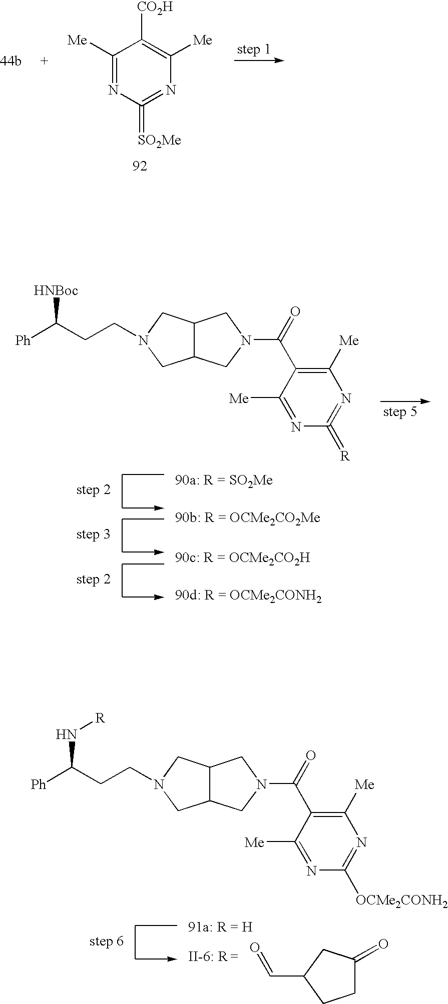

In step 1, 90a was prepared by acylation of 44b with 2-methanesulfonyl-4,6-dimethyl-pyrimidine-5-carboxylic acid as described in step 4 of example 8

Step 2—A mixture of 90a (0.374 mmol), 2-hydroxy-2-methyl-propionic acid methyl ester (0.5 mL) and K₂CO₃ (1.53 mmol) in DMF (2.0 mL) is heated at 70° C. overnight. The reaction mixture is cooled to RT and is partitioned between water and EtOAc. The organic layer is separated and is washed 3 times with water and once with brine, dried (MgSO₄), filtered and evaporated. The residue is purified via SiO₂ chromatography to afford 90b.

Step 3—A mixture of 90b and LiOH.H₂O (5 equiv) in 1:1 MeOH/water (4 mL) is stirred at RT overnight. The reaction is concentrated and the residue is purified via preparative TLC developed with DCM/MeOH/NH₄OH to afford 90c.

Step 4—Formation of the primary amide 90d is carried out as described in step 1 of example 7 except 56a is replaced by 5 equivalents of ammonia (1.0 M solution in dioxane). The resulting solution is stirred at RT until the reaction is complete. The resulting mixture is partitioned between EtOAc and H₂O. The combined organic extracts are combined, washed with H₂O and brine and evaporated. The crude product is purified by SiO₂ chromatography.

Step 5—Removal of the BOC protecting group to afford 91a is accomplished by stirring a solution of 90d in ice-cold DCM (5 mL) and TFA (5 mL) for 30 min. The reaction is warmed to RT and the volatile solvents evaporated. The residue is partitioned between EtOAc and saturated NaHCO3. The organic phase is washed with water and brine. The solution is dried filtered and evaporated to afford 91a.

Step 6—The amine 91a is converted to II-6 by the procedure described in step 5 of example 17 except 3-oxo-cyclohexanecarboxylic acid is replaced with 3-oxo-cyclopentanecarboxylic acid.

3-Oxo-cyclopentanecarboxylic acid ((S)-3-{5-[2-(carbamoylmethyl-methyl-amino)-4,6-dimethyl-pyrimidine-5-carbonyl]-hexahydro-pyrrolo[3,4-c]pyrrol-2-yl}-1-phenyl-propyl)-amide(II-5) is prepared analogously except in step 2, 2-hydroxy-2-methyl-propionic acid methyl ester is replaced by the amide of N-methyl glycine and steps 3 and 4 are omitted.

### EXAMPLE 20

### 2-((S)-1-(3-Fluoro-phenyl)-3-{5-[3,5-dimethyl-1-(6-trifluoromethyl-pyridazin-3-yl)-1H-pyrazole-4-carbonyl]-hexahydro-pyrrolo[3,4-c]pyrrol-2-yl}-propylcarbamoyl)-cyclopentanecarboxylic acid (II-1)

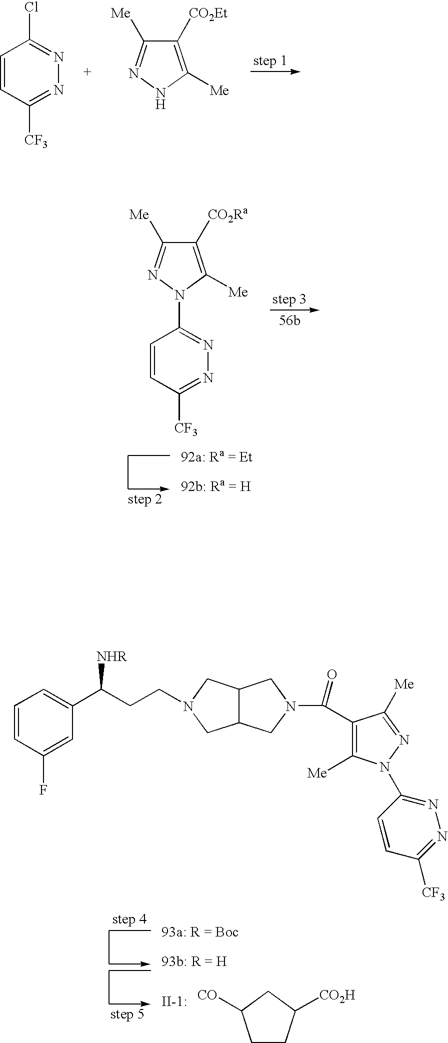 Step 1—To a solution of 3,5-dimethyl-1H-pyrazole-4-carboxylic acid ethyl ester (0.2 g, 1.19 mmol) in DMF (10 mL) cooled to 0° C. was added sequentially NaH (60% in mineral oil, 72 mg, 1.78 mmol) and 3-chloro-6-trifluoromethyl-pyridazine (0.22 g, 1.21 mmol; Tetrahedron 1999 55:15067-15070). The resulting mixture was stirred at RT for 3 h then partitioned between EtOAc and saturated aqueous NH₄CI. The layers were separated and the aqueous layer was extracted twice with EtOAc. The combined extracts were dried (Na₂SO₄), filtered and evaporated. The residue was purified via SiO₂ chromatography eluting with hexane/EtOAc to afford 0.228 g (62%) of 92a.
Step 2—To a solution of 92a (1.38 mmol) and 4 mL of H₂O was added a solution of KOH (0.155 g, 2.76 mmol) and 0.5 mL of H₂O. The mixture was stirred at 40° C. for 24 h them evaporated. The residue was partitioned between water and EtOAc. The aqueous layer was separated and adjusted to pH 2 with con HCl. The resulting precipitate was washed with H₂O and acetone and dried to afford 92b.
Step 3—To a suspension of 56b (0.03 g, 0.0878 mmol), 92b (0.0966 mmol), EDCI (0.020 g 0.105 mmol), HOBT monohydrate (0.016 g, 0.105 mmol), DMF (50 μL) and DCM (0.75 mL) was added diisopropylamine (70 μL, 0.4 mmol). The resulting solution was stirred at RT for 16 h. The resulting solution was partitioned between H₂O and EtOAc. The aqueous layer was twice extracted with EtOAc and the combined EtOAc extracts dried (Na₂SO₄), filtered and evaporated, The residue was purified by SiO₂ chromatography eluting with a gradient of 100% DCM to a linear gradient to a 1:1 mixture of DCM/(DCM/MeOH/NH₄Cl; 60/10/1), followed by isocratic elution with the 1:1 mixture for 10 min at a flow rate of 15 mL/min. to afford 0.0344 g(72.3%) of 93a.
Step 4—Removal of the BOC protecting group is carried out as described in step 5 of example 19 to afford 93b.
Step 5—A mixture of 93b (0.071 mmol) and trans-1,2-cyclopentanedicarboxylic anhydride (10.5 mg, 0.074 mmol) in DCM (1 mL) is stirred at RT for 18 h. The volatiles are evaporated and the residue is purified by SiO₂ chromatography to afford II-1.

2-[(S)-3-{5-[1-(5-Difluoromethyl-pyridin-2-yl)-3,5-dimethyl-1H-pyrazole-4-carbonyl]-hexahydro-pyrrolo[3,4-c]pyrrol-2-yl}-1-(3-fluoro-phenyl)-propylcarbamoyl]-cyclopentanecarboxylic acid (II-2) was prepared analogously except in step 1, 3-chloro-6-trifluoromethyl-pyridazine was replaced with 2-chloro-5-difluoromethylpyridine (CAS Reg. No. 71701-99-0)

### EXAMPLE 21

### 3-Oxo-cyclopentanecarboxylic acid {(S)-1-(3-chloro-phenyl)-3-[5-(3,5-dimethyl-1-pyrimidin-5-yl-1H-pyrazole-4-carbonyl)-hexahydro-pyrrolo[3,4-c]pyrrol-2-yl]-propyl}-amide (II-3)

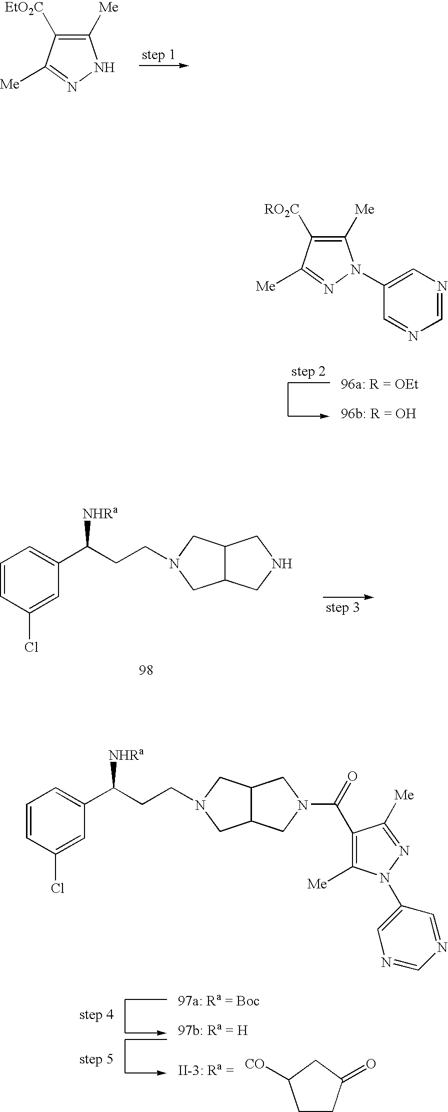 Step 1—N,N′-Dimethylethylenediamine (90 μL, 0.832 mmol) was added to a mixture of 3,5-dimethyl-1H-pyrazole-4-carboxylic acid ethyl ester (95, 1.4 g, 8.324 mmol), 5-bromopyrimidine (1.32 g, 8.303 mmol), CuI (0.16 g, 0.84 mmol) and K₂CO₃ (2.3 g, 16.64 mmol) in 1,4-dioxane (8 mL) that was maintained under an Ar atmosphere. The resulting mixture was stirred at 110° C. under Ar for 16 h. The reaction mixture was cooled to RT, diluted with DCM (50 mL) and filtered through a CELITE® and SiO₂ pad. The filter cake was rinsed with EtOAc and the filtrate was evaporated in vacuo. The residue was purified via SiO₂ chromatography eluting with hexane/EtOAc to afford the 0.150 g (7%) of 96a.
Step 2—A solution of KOH (77 mg, 1.38 mmol) in water (0.5 mL, plus 0.25 mL to rinse) was added to a solution of 96a (170 mg, 0.69 mmol) in EtOH (3 mL). The resulting mixture was stirred at 40° C. for 24 h, cooled to RT and evaporated in vacuo. The residue was partitioned between EtOAc and water and the resulting aqueous layer was separated and extracted with EtOAc. The aqueous layer was acidified to pH 4 with 3M HCl. The precipitate was filtered and rinsed with water to afford 0.086 g (57%) of 96b which was used for the next step without additional purification.

The amine 98 is prepared by the procedure steps 1-5 of example 8 except in step 1, (S)-3-tert-butoxycarbonylamino-3-(3-cyano-phenyl)-propionic acid was replaced with (S)-3-tert-butoxycarbonylamino-3-(3-chloro-phenyl)-propionic acid. Steps 3 and 4 were carried out by the procedures described in steps 3 and 4 of example 20. Step 5 is carried by the procedure described in step 6 of example 19 to afford II-3.

### EXAMPLE 22

### 3-(3-(3-Chloro-4-methyl-phenyl)-3-{3-[5-(4,6-dimethyl-pyrimidine-5-carbonyl)-hexahydro-pyrrolo[3,4-c]pyrrol-2-yl]-butyl}-ureido)-propionic acid methyl ester

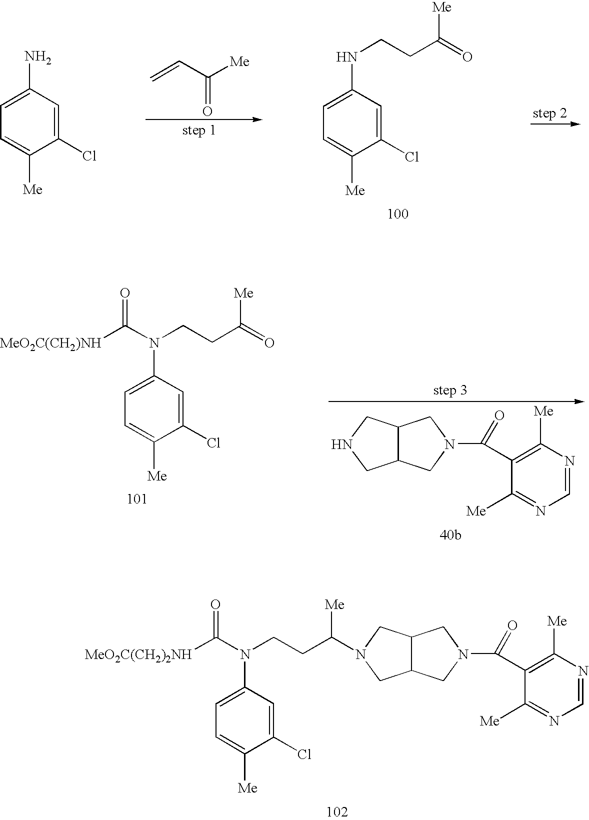 Step 1—To a mixture of 3-chloro-4-methyl-phenylamine (2.5 g, 17.65 mmol) and trifluoromethanesulfonimide (0.33 g, 1.20 mmol) in MeCN (20 mL) was added methyl vinyl ketone (1 mL, 12.05 mmol) at RT. After 1 h silica gel and Na₂CO₃ (200 mg) were added to the mixture and it was concentrated in vacuo. The crude product was purified by SiO₂ column chromatography eluting with n-hexane:EtOAc (4:1) to afford 1.3 g (51%) of 100: NMR (CDCl₃) δ 2.15 (s, 3H), 2.25 (s, 3H), 2.73 (t, 2H), 3.35 (t, 2H), 3.93 (br, 1H), 6.4 (dd, 1H), 6.6 (d, 1H), 6.98 (d, 1H).
Step 2—To a solution of 100 (1.3 g, 6.14 mmol) in DCM (30 mL) is added 3-isocyanato-propionic acid methyl ester (10 mmol) at 0° C. The solution is stirred at RT overnight. The mixture is diluted with DCM and is washed sequentially with H₂O, 2N HCl, saturated NaHCO₃ and brine. The organic layer is dried (Na₂SO₄), filtered and evaporated. The crude product is purified by SiO₂ column chromatography eluting with 5% MeOH/EtOAc to afford 101.
Step 3—To a solution of 101 (0.17 g, 0.48 mmol) in THF (7 mL) is added a solution of 76 (0.40 mmol) in DCM (7 mL). Titanium tetra-isopropoxide (0.26 mL, 0.89 mmol) is added to the mixture. The reaction is stirred for 40 min, NaBH(OAc)₃ (0.13 g, 0.61 mmol) is added to the mixture and stirring is continued at RT overnight. Saturated NaHCO₃ is added to the mixture and it is stirred for 10 min. The mixture is filtered through a CELITE® pad and the filtrate is extracted with DCM. The organic layer is dried (MgSO₄) and is purified by SiO₂ column chromatography eluting with DCM:MeOH:NH₄OH (150:10:1) afford 102.

### EXAMPLE 23

### 3-Oxo-cyclopentanecarboxylic acid {(S)-3-[5-(2,4-dimethyl-6-oxo-1,6-dihydro-pyridine-3-carbonyl)-hexahydro-pyrrolo[3,4-c]pyrrol-2-yl]-2-methyl-1-phenyl-propyl}-amide

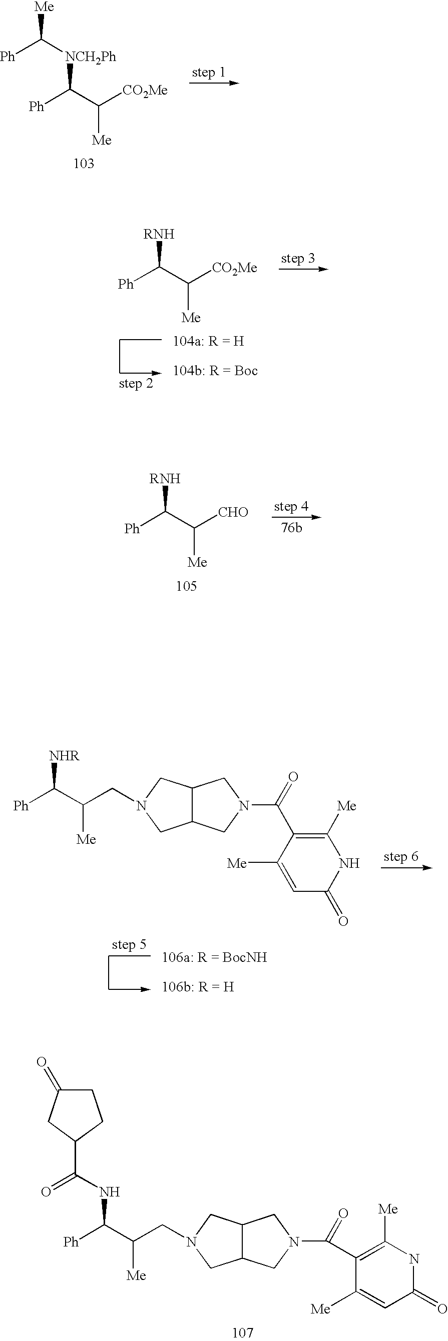 Step 1—A solution of (2R,3S,αR)3-[benzyl-(1-phenyl-ethyl)-amino]-2-methyl-3-phenyl-propionic acid methyl ester (103, 1.00 g, 2.58 mmol, prepared as described in J. Chem. Soc. Perkin Trans. 1 1994 1129) and MeOH:EtOAc:10% HCl solution (25 mL) containing Pd(OH)₂—C (0.50 g) and hydrogenated (1 atm) for 24 h. The reaction mixture was filtered through a CELITE® pad to remove the catalyst. The filtrate was concentrated in vacuo and the residue partitioned between Et₂O (40 mL) and saturated NaHCO₃ solution (25 mL). The organic layer was dried (MgSO₄) and concentrated in vacuo to afford 408 mg (80%) of 104a as a pale yellow liquid: ms (ES+) m/z 194 (M+H)⁺.
Step 2—A solution of (2R,3S)-3-amino-2-methyl-3-phenyl-propionic acid methyl ester (104a, 400 mg, 2.06 mmol) in THF (5 mL) was cooled to 0° C. A cold solution of NaOH (166 mg, 4.14 mmol) in H₂O (3.75 mL) was added to the above solution followed by a solution of (BOC)₂O in THF (2.5 mL) and the mixture stirred at RT for 5 h. The reaction mixture was extracted with EtOAc (2×50 mL) and the combined organic extracts were dried (MgSO₄) and concentrated in vacuo to afford 104b as a waxy solid: ms (ES+) m/z 237 (M−C₄H₈)⁺.
Step 3—To a solution of 104b (355 mg, 1.21 mmol) in DCM (20 mL) cooled to −78° C. was added DIBAL-H (2.42 mL of 1 M DCM solution, 2.42 mmol) dropwise at such a rate to maintain the temperature below −70° C. After 2 h the reaction was quenched by the slow addition of MeOH (2 mL) and then allowed to warm to RT. The reaction mixture was filtered through a CELITE® pad. The filtrate was dried (Na₂SO₄) and concentrated in vacuo. The crude product was purified by flash chromatography on silica eluting with EtOAc:hexane (1:3) to afford 105 as a white solid: ¹H-NMR showed this material to be a 1:1.38 ratio of diastereomers.
Step 4—To a solution of 105 (197 mg, 0.75 mmol) and 76b (0.75 mmol) in DCM (16 mL) containing HOAc (0.11 ImL) is added NaBH(OAc)₃ (191 mg, 0.90 Mmol) in 1 portion and the reaction is stirred for 18 h at RT. The reaction is quenched by the addition of 10% K₂CO₃ solution (10 mL) and is stirred for 20 min. The product is extracted with DCM (2×20 mL) and the combined extracts are dried (Na₂SO₄) and concentrated in vacuo. The crude product is purified by flash chromatography on silica eluting with DCM/7.5% MeOH (containing 2% NH₄OH) to afford 106a.
Step 5—A solution of 106a (258 mg, 0.52 mmol) dissolved in 10 M HCl in MeOH (8 mL) is heated at 65° C. for 2 h. The MeOH is evaporated under reduced pressure and the residue is cautiously partitioned between DCM (25 mL) and 20% K₂CO₃ solution (15 mL). The aqueous layer is re-extracted with DCM (2×20 mL). The combined extracts is dried (Na₂SO₄) and concentrated in vacuo to afford 106b.
Step 6—To a solution of 106b (0.050 mmol) and DIPEA (0.03 mL) is added cyclopropanecarbonyl chloride (6.8 μL, 7.8 mg, 0.075 mmol) and the resulting mixture is stirred at RT for 18 h. The reaction mixture is concentrated in a stream of N₂ and purified by reverse phase HPLC to afford 107.

### EXAMPLE 24

### 3-Acetylamino-cyclobutanecarboxylic acid [3-[5-(4,6-dimethyl-pyrimidine-5-carbonyl)-hexahydro-pyrrolo[3,4-c]pyrrol-2-yl]-1-(3-fluoro-phenyl)-propyl]-amide (I-39) and 3-acetylamino-cyclobutanecarboxylic acid [3-[5-(4,6-dimethyl-pyrimidine-5-carbonyl)-hexahydro-pyrrolo[3,4-c]pyrrol-2-yl]-1-(3-fluoro-phenyl)-propyl]-amide (I-41)

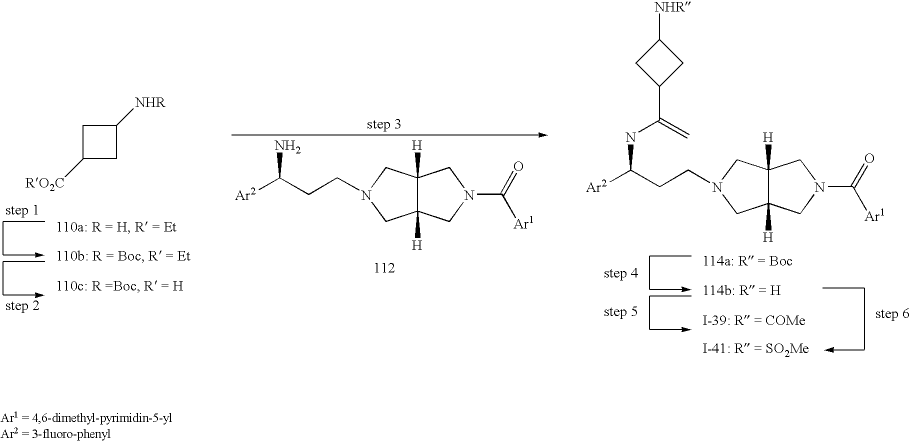 Step 1—3-Amino-cyclobutanecarboxylic acid ethyl ester (110a, 0.68 g, 4.75 mmol, CAS Reg No. 74307-73-6) and Boc₂O (1.24 g, 5.70 mmol) were dissolved in EtOH (120 mL) with stirring. Stirring was continued for 24 h at RT. The reaction mixture was concentrated and residue purified by flash chromatography eluting with 20% EtOAc/hexanes to afford 1.03 g (90%) of 110b as a colorless liquid which solidifies upon standing: MS m/z=266 (M+Na)⁺.
Step 2—To a solution of 110b (0.4 g, 1.64 mmol) in THF (6.5 mL) was added a solution of LiOH monohydrate (0.138 g, 3.28 mmol) in water (6.5 mL) and the mixture was stirred overnight at RT. The reaction mixture was washed with 20 mL of ether and the aqueous layer acidified with 1M KHSO₄. The product extracted with EtOAc (3×20 mL), dried (MgSO₄) and concentrated in vacuo to afford 0.335 g (95%) of 110c as a white crystalline solid: MS m/z (no molecular ion).
Step 3—To a mixture of 110c (0.155 g, 0.72 mmol) and {5-[3-amino-3-(3-fluoro-phenyl)-propyl]-hexahydro-pyrrolo[3,4-c]pyrrol-2-yl}-(4,6-dimethyl-pyrimidin-5-yl)-methanone (112, 0.239 g, 0.60 mmol) in DCM (9 mL) at RT was added sequentially EDCI (0.15 g, 0.78 mmol), HOBt (0.106 g, 0.60 mmol) and DIPEA (0.32 mL, 1.80 mmol). The mixture was stirred overnight at RT. The reaction mixture was stirred with 10% K₂CO₃ solution for 30 min, extracted with DCM (3×), the combined extracts dried (MgSO₄) and concentrated in vacuo. The crude product was purified by SiO₂ chromatography eluting with 8% MeOH in DCM to afford 0.281 g (78%) of 114a as a white foam, MS m/z=595 (M+H)⁺.
Step 4—To a solution of 114a (0.281 g, 0.47 mmol) in DCM (6.5 mL) was added TFA (1.6 mL) and the mixture was stirred at RT overnight. The solvents were evaporated, the residue suspended in toluene and re-evaporated (2×). The residue was dissolved in MeOH (10 mL) and stirred with PL-CO₃²⁻ (1 g, 2.16 mmol, 4.6 equiv; polymer-supported carbonate available from Polymer Laboratories, Ltd.) for 2 h. The resin was filtered off and washed with MeOH. The combined filtrates were evaporated and the 114b thus obtained was used in the next step without further purification.
Step 5—A mixture of 114b (0.100 g, 0.20 mmole), acetic anhydride (0.025 g, 0.24 mmol) and TEA (0.025 g, 0.24 mmole) in acetone (5 mL) was stirred for 30 min at RT. The reaction mixture was concentrated in vacuo and the crude product was purified by flash chromatography eluting with 4% MeOH (containing 10% NH₄OH) in DCM to afford 0.095 g of I-40 as a white foam, MS m/z=537 (M+H)⁺. Calculated for C₂₉H₃₇FN₆O₃.0.65CH₂Cl₂: C, 60.17; H, 6.52; N, 14.20. Found: C, 60.25; H, 6.48; N, 14.59.
Step 6—A mixture of 114b (0.133 g, 0.26 mmole), methanesulfonyl chloride (0.037 g, 0.32 mmol) and DIPEA (0.042 g, 0.32 mmole) in DCM (5 mL) was stirred for overnight at RT. The reaction mixture was stirred with 10% K₂CO₃ solution for 30 min, extracted with DCM (3×), and the combined extracts dried (MgSO₄) and concentrated in vacuo. The crude product was not pure after flash chromatography eluting with 6% MeOH (containing 10% NH₄OH) in DCM. Final purification was achieved by reverse phase HPLC to afford I-41: MS (ES+) m/z 573 (M+H)⁺.

### EXAMPLE 25

### 3-(Acetyl-methyl-amino)-cyclobutanecarboxylic acid [3-[5-(4,6-dimethyl-pyrimidine-5-carbonyl)-hexahydro-pyrrolo[3,4-c]pyrrol-2-yl]-1-(3-fluoro-phenyl)-propyl]-amide (I-40) and 3-(Methanesulfonyl-methyl-amino)-cyclobutanecarboxylic acid [3-[5-(4,6-dimethyl-pyrimidine-5-carbonyl)-hexahydro-pyrrolo[3,4-c]pyrrol-2-yl]-1-(3-fluoro-phenyl)-propyl]-amide (I-42)

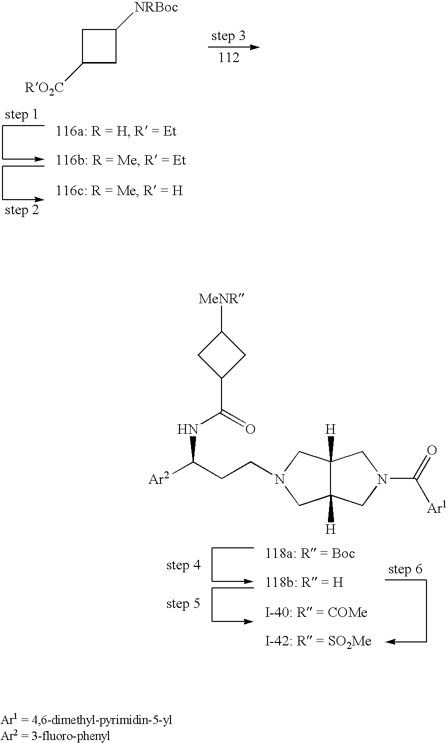 Step 1—To a solution of 116a (0.444 g, 1.82 mmol) in DMF (4.5 mL) was added NaH (80 mg of 60% dispersion in mineral oil, 2.0 mmol) and the mixture stirred for 30 min at RT. The reaction was cooled in an ice bath and MeI (0.31 g, 2.19 mmol) was added with stirring. Stirring continued overnight at RT. The reaction mixture was concentrated under high vacuum and residue partitioned between saturated NH₄Cl solution and DCM. The aqueous layer was extracted with DCM (3×), the combined extracts were dried (MgSO₄) and concentrated in vacuo. The crude product was purified by flash chromatography eluting with 20% EtOAc/hexanes to afford 0.267 g (56%) of 116b as a colorless liquid: MS m/z=258 (M+H)⁺.

3-Methylamino-cyclobutanecarboxylic acid [3-[5-(4,6-dimethyl-pyrimidine-5-carbonyl)-hexahydro-pyrrolo[3,4-c]pyrrol-2-yl]-1-(3-fluoro-phenyl)-propyl]-amide (116b) was prepared as described in steps 2, 3 and 4 of example.

3-(Acetyl-methyl-amino)-cyclobutanecarboxylic acid [3-[5-(4,6-dimethyl-pyrimidine-5-carbonyl)-hexahydro-pyrrolo[3,4-c]pyrrol-2-yl]-1-(3-fluoro-phenyl)-propyl]-amide (I-40) was prepared from 116b following the procedure described on step 5 of example 24. MS m/z=551 (M+H)⁺.

3-(Methanesulfonyl-methyl-amino)-cyclobutanecarboxylic acid [3-[5-(4,6-dimethyl-pyrimidine-5-carbonyl)-hexahydro-pyrrolo[3,4-c]pyrrol-2-yl]-1-(3-fluoro-phenyl)-propyl]-amide (I-42) was prepared from 116b following the procedure described on step 6 of example 24 MS m/z=587 (M+H)⁺.

### EXAMPLE 26

### Hexanoic acid 3-[3-[5-(4,6-dimethyl-pyrimidine-5-carbonyl)-hexahydro-pyrrolo[3,4-c]pyrrol-2-yl]-1-(3-fluoro-phenyl)-propylcarbamoyl]-cyclopentyl ester (I-43)

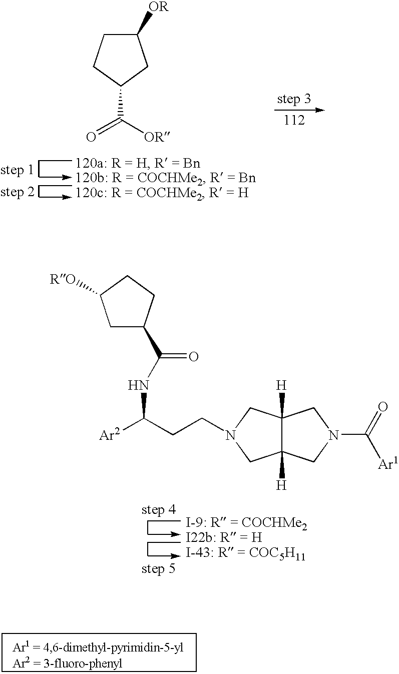 Step 1—To a solution of (1R,3R)-3-hydroxy-cyclopentanecarboxylic acid benzyl ester (120a, 0.315 g, 1.43 mmol, CAS Reg. No. 128095-32-9) and pyridine (0.339 g, 4.29 mmol) in DCM (10 mL) was added isobutyryl chloride (0.304 g, 2.86 mmol) under N₂ and the mixture stirred at RT overnight. The reaction mixture was washed with 2N HCl (10 mL), brine and saturated NaHCO₃ solution. The organic layer was dried (MgSO₄) and concentrated. The crude residue was purified by flash chromatography eluting with 0 to 30% EtOAc/hexanes to afford 0.4 g (96%) of 120b as a colorless liquid.
Step 2—A stirred suspension of 120b (0.4 g, 1.37 mmol) and 10% Pd/C (0.045 g, catalytic amount) in EtOH (20 mL) was maintained under a H₂ atmosphere for 2 h with a H₂ filled balloon. The catalyst was filtered and the filtrate evaporated to afford 0.257 g (93%) of 120c as a colorless liquid.
Step 3—Isobutyric acid 3-[3-[5-(4,6-dimethyl-pyrimidine-5-carbonyl)-hexahydro-pyrrolo[3,4-c]pyrrol-2-yl]-1-(3-fluoro-phenyl)-propylcarbamoyl]-cyclopentyl ester (I-9) was prepared from 112 and 120c using the procedure described in step 3 of example 24: MS m/z=580 (M+H)⁺.
Step 4—Hydrolysis of I-9 was carried out by the procedure described in step 2 of example 24 to afford 122b: MS m/z=510 (M+H)⁺.
Step 5—A mixture of 122b (0.020 g, 0.039 mmol), diisopropylcarbodiimide (0.0071 g, 0.049 mmol), hexanoic acid (0.0073 g, 0.049 mmol) and DMAP (0.0075 g, 0.059 mmol) in DCM (1.5 mL) was stirred at RT overnight. The solvents were evaporated and the residue was purified by reverse phase HPLC.

### EXAMPLE 27

### CCR5-Mediated CCF Assay

CCF assay was performed as described before (C. Ji, J. Zhang, N. Cammack and S. Sankuratri, J. Biomol. Screen. 2006 11(6):652-663). Hela-R5 cells (express gp160 from R5-tropic virus and HIV-1 Tat) were plated in 384 well white culture plates (BD Bioscience, Palo Alto, Calif.) at 7.5×10³ cells per well in phenol red-free Dulbecco's Modified Eagle Medium (DMEM) supplemented with 10% FBS, 1× Pen-Strep, 300 μg/mL G418, 100 μg/mL hygromycin, and 1 μg/mL Doxycycline (Dox) (BD Bioscience, Palo Alto, Calif.), using Multimek (Beckman, Fullerton, Calif.) and incubated at 37° C. overnight to induce the expression of gp160. Ten μL diluted compounds in medium containing 5% DMSO were added to the cells, followed by the addition of CEM-NKr-CCR5-Luc (obtained from NIH AIDS Research & Reference Reagents Program) that expresses CD4 and CCR5 and carries a HIV-2 long terminal repeat (LTR)-driven luciferase reporter gene at 1.5×10⁴ cells/15 μL/well and incubated for 24 hrs. At the end of co-culture, 15 μL of Steady-Glo luciferase substrate was added into each well, and the cultures were sealed and gently shaken for 45 min. The luciferase activity were measured for 10 sec per well as luminescence by using 16-channel TopCount NXT (PerkinElmer, Shelton, Conn.) with 10 min dark adaptation and the readout is count per second (CPS). For the drug interaction experiments, small molecule compounds or antibodies were serially diluted in serum-free and phenol red-free RPMI containing 5% DMSO (CalBiochem, La Jolla, Calif.) and 1× Pen-Strep. Five μL each of the two diluted compound or mAb to be tested for drug-drug interactions were added to the Hela-R5 cells right before the addition of target cells. The checker board drug combinations at various concentrations were carried out as shown in FIG. 1A.

TABLE 3

| Cpd. No. | IC₅₀ (μM) |
|---|---|
| I-4 | 0.0098 |
| I-9 | 0.0042 |
| I-23 | 0.0074 |
| I-24 | 0.0065 |
| I-31 | 0.0035 |
| I-33 | 0.0017 |

### EXAMPLE 28

Pharmaceutical compositions containing the subject compounds for administration via several routes are prepared as described in this Example.

Composition for Oral Administration (A)

| Ingredient | % wt./wt. |
|---|---|
| Active ingredient | 20.0% |
| Lactose | 79.5% |
| Magnesium stearate | 0.5% |

The ingredients are mixed and dispensed into capsules containing about 100 mg each; one capsule would approximate a total daily dosage.

Composition for Oral Administration (B)

| Ingredient | % wt./wt. |
|---|---|
| Active ingredient | 20.0% |
| Magnesium stearate | 0.5% |
| Crosscarmellose sodium | 2.0% |
| Lactose | 76.5% |
| PVP (polyvinylpyrrolidine) | 1.0% |

The ingredients are combined and granulated using a solvent such as methanol. The formulation is then dried and formed into tablets (containing about 20 mg of active compound) with an appropriate tablet machine.

Composition for Oral Administration (C)

| Ingredient | % wt./wt. | |
|---|---|---|
| Active compound | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.5 | g |
| Sorbitol (70% solution) | 12.85 | g |
| Veegum K (Vanderbilt Co.) | 1.0 | g |
| Flavoring | 0.035 | ml |
| Colorings | 0.5 | mg |
| Distilled water | q.s. to 100 | ml |

The ingredients are mixed to form a suspension for oral administration.

Parenteral Formulation (D)

| Ingredient | % wt./wt. | |
|---|---|---|
| Active ingredient | 0.25 | g |
| Sodium Chloride | qs to make isotonic | |
| Water for injection to | 100 | ml |

The active ingredient is dissolved in a portion of the water for injection. A sufficient quantity of sodium chloride is then added with stirring to make the solution isotonic. The solution is made up to weight with the remainder of the water for injection, filtered through a 0.2 micron membrane filter and packaged under sterile conditions.

Suppository Formulation (E)

| Ingredient | % wt./wt. |
|---|---|
| Active ingredient | 1.0% |
| Polyethylene glycol 1000 | 74.5% |
| Polyethylene glycol 4000 | 24.5% |

The ingredients are melted together and mixed on a steam bath, and poured into molds containing 2.5 g total weight.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

We claim:

1. A compound according to formula I wherein: 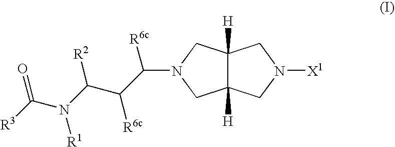
one of R¹ and R² is phenyl optionally substituted with one to four substituents selected independently in each incidence from the group consisting of halogen, C₁₋₆ alkyl, cyano and C₁₋₆ alkoxy; and, the other of R¹ and R² is hydrogen;
R⁵ is hydroxy, NR^{6a}R^{6b}, C₁₋₆ alkoxy or benzyloxy;
R⁶ is hydrogen, C₁₋₆ alkyl, C₁₋₃ haloalkyl, C₁₋₆ hydroxyalkyl or oxo-C₁₋₆ alkyl;
R⁶a, R^{6b}, R^{6c} and R^{6d} are independently hydrogen or C₁₋₃ alkyl with the proviso that at least one of R^{6c} is hydrogen;
X¹ is selected from the group consisting of (i)-(xiii) and (xiv):
wherein
X² is N or CH;
A¹ is C₁₋₆ straight or branched alkylene optionally substituted by a phenyl ring or phenylene;
m is zero to two; 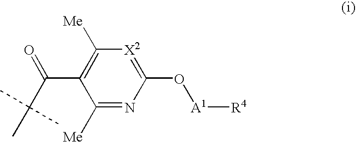
wherein R⁴ is C(═O)R⁵ or hydrogen; 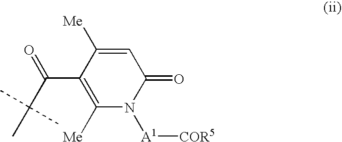
with the proviso that A¹ is other than phenylene; 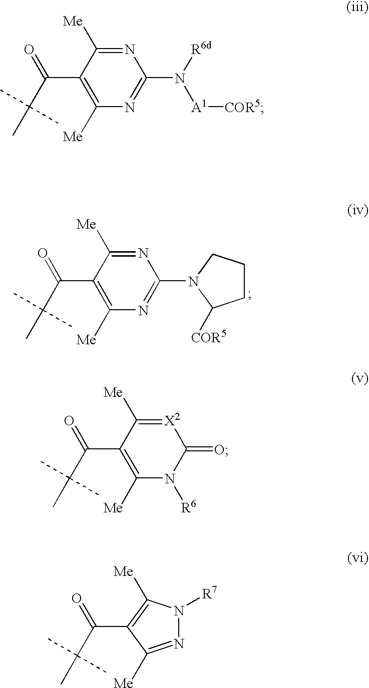
wherein:
R⁷ is C₃₋₇ cycloalkyl, (CH₂)ₙCOR⁵, heteroaryl selected from the group consisting of pyridine, pyrimidine, pyrazine and pyridazine said heteroaryl optionally substituted with C₁₋₃ alkyl or C₁₋₃ haloalkyl;
n is 1 to 3; 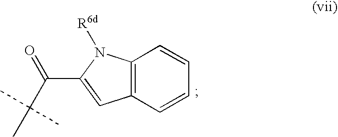 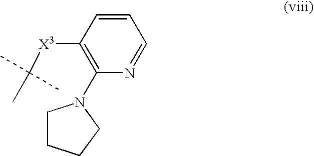
wherein X³ is —S(O)₂— or —C(O)—; 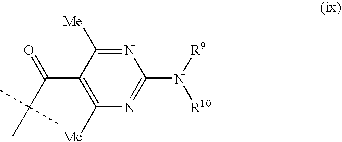
wherein
R⁹ and R¹⁰ are (A) together a group (CH₂)₂X⁴(CH₂)₂, (CH₂)₂CH(R¹²)CH₂, or (CH₂)₂SO₂; or, (B) independently R¹⁰ is hydrogen or C₁₋₃ alkyl and R⁹ is —SO₂C₁₋₆ alkyl, C₁₋₆ hydroxyalkyl, xA, xB or xC; 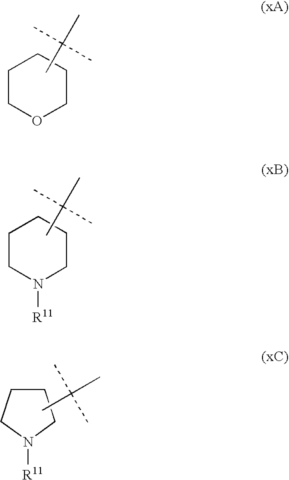
X⁴ is O, S(O)ₘ, NR¹¹ or CH(NHSO₂C₁₋₆ alkyl);
R¹¹ is R^{6d}, —C(O)C₁₋₆ alkyl, S(O)₂C₁₋₆ alkyl;
R¹² is hydrogen, hydroxyl or C₁₋₁₀ acyloxy;
m is zero to two; and, 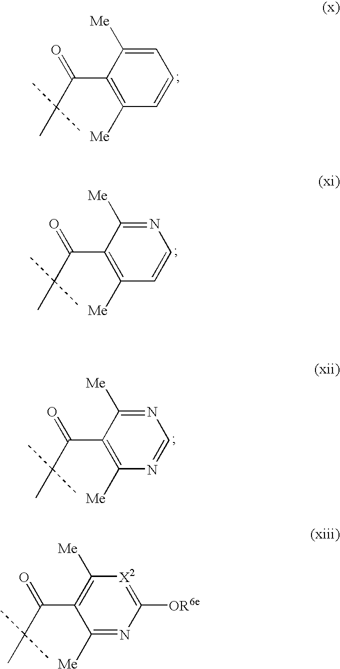
wherein R^{6e} is C₁₋₆ hydroxyalkyl or oxo-C₁₋₆ alkyl; and, 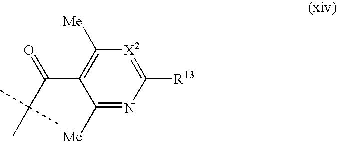
wherein R¹³ is C₃₋₅ cycloalkyl or C₁₋₃ alkynyl;
R³ is selected from the group consisting of (i), (ii), (iii) (iv) and (v) wherein:
(i) C₃₋₇ cycloalkyl substituted one or more substituents selected from the group consisting of C₁₋₆ alkoxy, CO₂R^{6d}, CONR^{6a}R^{6b}, fluorine, —NR^{6d}CO C₁₋₃alkyl, —NR^{6d}SO₂ C₁₋₃ alkyl, and C₁₋₁₀ acyloxy or two hydrogens on the same carbon together are replaced by oxygen (oxo) provided that R³ is not 4-oxo-cyclohexyl or 3-oxo-cyclobutyl and when the cycloalkyl is substituted with fluorine, R² is meta-cyano-phenyl; 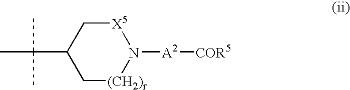
wherein:
A² is C₁₋₆ straight or branched alkylene wherein one carbon atom can optionally be replaced by —O—, —S(O)ₘ—, or NR⁵ providing the carbon replaced is not bonded to the heterocyclic nitrogen or the terminal carboxy moiety or A² is absent and R⁵ is tert-butyl;
X⁵ is C(═O) or CH₂;
r is zero or one; 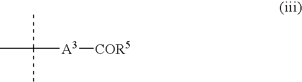
wherein A³ is C₁₋₆ alkylene said alkylene optionally substituted with C₅₋₇ cycloalkyl or A³-COR⁵ together represent NH(CH₂)ₙCOR⁵; n is one to three; 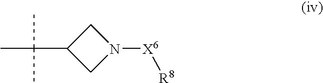
wherein:
X⁶ is C(O)R⁸ or S(O)₂C₁₋₆ alkyl;
R⁸ is C₁₋₆ alkyl, C₁₋₆ haloalkyl, C₃₋₇ cycloalkyl, C₃₋₇ cycloalkyl-C₁₋₃ alkyl; C₁₋₆ alkoxy or C₁₋₆ alkylamino;
with the proviso that when R³ is (iv), X¹ is not (x), (xi) or (xii);
(v) phenylamine optionally substituted with —SO₂NH₂; and,
pharmaceutically acceptable salts, hydrates and solvates.

2. A compound according to claim 1 wherein:
R⁶ is hydrogen or C₁₋₆ alkyl;
X¹ is (i) to (xi) or (xii);
R⁹ and R¹⁰ are (A) together a group (CH₂)₂X⁴(CH₂)₂ or (B) R¹⁰ is hydrogen or C₁₋₃ alkyl and R⁹ is —SO₂C₁₋₆ alkyl, xA or xB;
R³ is selected from the group consisting of (i), (ii), (iii) and (iv) wherein (i) C₃₋₇ cycloalkyl substituted with C₁₋₆ alkoxy, CO₂R^{6d}, CONR^{6a}R^{6b} or two hydrogens on the same carbon together are replaced by oxygen (oxo) with the proviso that R³ is not 4-oxo-cyclohexyl or 3-oxo-cyclobutyl.

3. A compound according to claim 1 wherein X¹ is (x), (xi) or (xii).

4. A compound according to claim 3 wherein R³ is C₃₋₇ cycloalkyl substituted with C₁₋₆ alkoxy, CO₂R^{6d}, CONR^{6a}R^{6b} or two hydrogens on the same carbon together are replaced by oxygen (oxo) wherein R^{6a} and R^{6b} are independently R⁶ with the proviso that R³ is not 4-oxo-cyclohexyl or 3-oxo-cyclobutyl.

5. A compound according to claim 3 wherein R³ is cyclopentyl or cyclohexyl substituted with CO₂R^{6d}, 3-oxo-cyclopentyl or 3-oxo-cyclohexyl.

6. A compound according to claim 3 wherein R³ is (ii).

7. A compound according to claim 6 wherein:
A¹ is C₁₋₆ straight or branched alkylene;
X⁵ is CH₂; and,
r is one.

8. A compound according to claim 1 wherein X¹ is selected from the group consisting of (i), (ii), (iii), (iv), (v), (vi), (vii), (viii), (ix), (xiii) or (xiv).

9. A compound according to claim 8 wherein X¹ is (vi) and R⁷ is heteroaryl selected from the group consisting of pyridine, pyrimidine, pyrazine and pyridazine said heteroaryl optionally substituted with C₁₋₃ alkyl or C₁₋₃ haloalkyl.

10. A compound according to claim 8 wherein X¹ is (v) and R⁶ is C₁₋₃ alkyl.

11. A compound according to claim 1 wherein X¹ is (i) or (iii), R⁵ is hydroxyl, C₁₋₆ alkoxy, or NR^{6a}R^{6b} and R^{6a} and R^{6b} are hydrogen.

12. A compound according to claim 1 which compound, or a pharmaceutically acceptable salt thereof, is selected from the group consisting of:
4-(3-(3-chloro-4-methyl-phenyl)-3-{3-[5-(4,6-dimethyl-pyrimidine-5-carbonyl)-hexahydro-pyrrolo[3,4-c]pyrrol-2-yl]-propyl}-ureido)-butyric acid, TFA salt;
2-[4-((3-chloro-4-methyl-phenyl)-{3-[5-(4,6-dimethyl-pyrimidine-5-carbonyl)-hexahydro-pyrrolo[3,4-c]pyrrol-2-yl]-propyl}-carbamoyl)-piperidin-1-yl]-3-methyl-butyric acid ethyl ester, TFA salt;
[4-((3-chloro-4-methyl-phenyl)-{3-[5-(4,6-dimethyl-pyrimidine-5-carbonyl)-hexahydro-pyrrolo[3,4-c]pyrrol-2-yl]-propyl}-carbamoyl)-piperidin-1-yl]-acetic acid TFA salt;
(1S,3R)-3-((3-chloro-4-methyl-phenyl)-{3-[5-(2,6-dimethyl-benzoyl)-hexahydro-pyrrolo[3,4-c]pyrrol-2-yl]-propyl}-carbamoyl)-cyclopentanecarboxylic acid, TFA salt;
4-[4-((3-chloro-4-methyl-phenyl)-{3-[5-(4,6-dimethyl-pyrimidine-5-carbonyl)-hexahydro-pyrrolo[3,4-c]pyrrol-2-yl]-propyl}-carbamoyl)-piperidin-1-yl]-butyric acid, TFA salt;
{2-[4-((3-chloro-4-methyl-phenyl)-{3-[5-(4,6-dimethyl-pyrimidine-5-carbonyl)-hexahydro-pyrrolo[3,4-c]pyrrol-2-yl]-propyl}-carbamoyl)-piperidin-1-yl]-2-oxo-ethoxy}-acetic acid, TFA salt;
(1R,2S)-2-((3-chloro-4-methyl-phenyl)-{3-[5-(4,6-dimethyl-pyrimidine-5-carbonyl)-hexahydro-pyrrolo[3,4-c]pyrrol-2-yl]-propyl}-carbamoyl)-cyclopentanecarboxylic acid, TFA salt;
3-(3-(3-chloro-4-methyl-phenyl)-3-{3-[5-(4,6-dimethyl-pyrimidine-5-carbonyl)-hexahydro-pyrrolo[3,4-c]pyrrol-2-yl]-propyl}-ureido)-propionic acid, TFA salt;
isobutyric acid 3-[3-[5-(4,6-dimethyl-pyrimidine-5-carbonyl)-hexahydro-pyrrolo[3,4-c]pyrrol-2-yl]-1-(3-fluoro-phenyl)-propylcarbamoyl]-cyclopentyl ester;
3-[4-((3-chloro-4-methyl-phenyl)-{3-[5-(4,6-dimethyl-pyrimidine-5-carbonyl)-hexahydro-pyrrolo[3,4-c]pyrrol-2-yl]-propyl}-carbamoyl)-piperidin-1-yl]-propionic acid tert-butyl ester, TFA salt;
4-[4-((3-chloro-4-methyl-phenyl)-{3-[5-(4,6-dimethyl-pyrimidine-5-carbonyl)-hexahydro-pyrrolo[3,4-c]pyrrol-2-yl]-propyl}-carbamoyl)-piperidin-1-yl]-butyric acid tert-butyl ester, TFA salt;
3-[4-((3-chloro-4-methyl-phenyl)-{3-[5-(4,6-dimethyl-pyrimidine-5-carbonyl)-hexahydro-pyrrolo[3,4-c]pyrrol-2-yl]-propyl}-carbamoyl)-piperidin-1-yl]-propionic acid, TFA salt;
2-{(S)-3-[5-(2,6-dimethyl-benzoyl)-hexahydro-pyrrolo[3,4-c]pyrrol-2-yl]-1-phenyl-propylcarbamoyl}-3-methyl-butyric acid, TFA salt;
2-cyclohexyl-N-{(S)-3-[5-(2,6-dimethyl-benzoyl)-hexahydro-pyrrolo[3,4-c]pyrrol-2-yl]-1-phenyl-propyl}-malonamic acid, TFA salt;
3-Oxo-cyclopentanecarboxylic acid (3-chloro-4-methyl-phenyl)-{3-[5-(4,6-dimethyl-pyrimidine-5-carbonyl)-hexahydro-pyrrolo[3,4-c]pyrrol-2-yl]-propyl}-amide, TFA salt;
3-oxo-cyclopentanecarboxylic acid {3-[5-(4,6-dimethyl-pyrimidine-5-carbonyl)-hexahydro-pyrrolo[3,4-c]pyrrol-2-yl]-propyl}-phenyl-amide, TFA salt;
2-oxo-cyclopentanecarboxylic acid [(S)-3-[5-(4,6-dimethyl-pyrimidine-5-carbonyl)-hexahydro-pyrrolo[3,4-c]pyrrol-2-yl]-1-(3-fluoro-phenyl)-propyl]-amide;
[4-((3-chloro-4-methyl-phenyl)-{3-[5-(4,6-dimethyl-pyrimidine-5-carbonyl)-hexahydro-pyrrolo[3,4-c]pyrrol-2-yl]-propyl}-carbamoyl)-2-oxo-pyrrolidin-1-yl]-acetic acid, TFA salt;
2-[4-((3-chloro-4-methyl-phenyl)-{3-[5-(4,6-dimethyl-pyrimidine-5-carbonyl)-hexahydro-pyrrolo[3,4-c]pyrrol-2-yl]-propyl}-carbamoyl)-2-oxo-pyrrolidin-1-yl]-propionic acid, TFA salt;
2-cyclohexyl-N-{(S)-3-[5-(2,6-dimethyl-benzoyl)-hexahydro-pyrrolo[3,4-c]pyrrol-2-yl]-1-phenyl-propyl}-malonamic acid methyl ester;
3,3-difluoro-cyclobutanecarboxylic acid {(S)-1-(3-cyano-phenyl)-3-[5-(4,6-dimethyl-pyrimidine-5-carbonyl)-hexahydro-pyrrolo[3,4-c]pyrrol-2-yl]-propyl}-amide;
4,4-difluoro-cyclohexanecarboxylic acid {(S)-1-(3-cyano-phenyl)-3-[5-(4,6-dimethyl-pyrimidine-5-carbonyl)-hexahydro-pyrrolo[3,4-c]pyrrol-2-yl]-propyl}-amide;
3-oxo-cyclopentanecarboxylic acid [(S)-3-[5-(2,4-dimethyl-pyridine-3-carbonyl)-hexahydro-pyrrolo[3,4-c]pyrrol-2-yl]-1-(3-fluoro-phenyl)-propyl]-amide;
3-oxo-cyclohexanecarboxylic acid [(S)-3-[5-(4,6-dimethyl-pyrimidine-5-carbonyl)-hexahydro-pyrrolo[3,4-c]pyrrol-2-yl]-1-(3-fluoro-phenyl)-propyl]-amide;
[4-((3-chloro-4-methyl-phenyl)-{3-[5-(4,6-dimethyl-pyrimidine-5-carbonyl)-hexahydro-pyrrolo[3,4-c]pyrrol-2-yl]-propyl}-carbamoyl)-piperidin-1-yl]-acetic acid tert-butyl ester;
4-((3-chloro-4-methyl-phenyl)-{3-[5-(4,6-dimethyl-pyrimidine-5-carbonyl)-hexahydro-pyrrolo[3,4-c]pyrrol-2-yl]-propyl}-carbamoyl)-cyclohexanecarboxylic acid, TFA salt;
[4-((3-chloro-4-methyl-phenyl)-{3-[5-(2,4-dimethyl-pyridine-3-carbonyl)-hexahydro-pyrrolo[3,4-c]pyrrol-2-yl]-propyl}-carbamoyl)-2-oxo-piperidin-1-yl]-acetic acid, TFA salt;
3-(3-(3-chloro-4-methyl-phenyl)-3-{3-[5-(4,6-dimethyl-pyrimidine-5-carbonyl)-hexahydro-pyrrolo[3,4-c]pyrrol-2-yl]-propyl}-ureido)-benzenesulfonamide;
(1S,2S)-2-((3-chloro-4-methyl-phenyl)-{3-[5-(2,6-dimethyl-benzoyl)-hexahydro-pyrrolo[3,4-c]pyrrol-2-yl]-propyl}-carbamoyl)-cyclohexanecarboxylic acid, TFA salt;
(1R,3S)-3-((3-chloro-4-methyl-phenyl)-{3-[5-(2,6-dimethyl-benzoyl)-hexahydro-pyrrolo[3,4-c]pyrrol-2-yl]-propyl}-carbamoyl)-cyclohexanecarboxylic acid, TFA salt;
4-((3-chloro-4-methyl-phenyl)-{3-[5-(4,6-dimethyl-pyrimidine-5-carbonyl)-hexahydro-pyrrolo[3,4-c]pyrrol-2-yl]-propyl}-carbamoyl)-cyclohexanecarboxylic acid methyl ester, TFA salt;
4-((3-chloro-4-methyl-phenyl)-{3-[5-(4,6-dimethyl-pyrimidine-5-carbonyl)-hexahydro-pyrrolo[3,4-c]pyrrol-2-yl]-propyl}-carbamoyl)-cyclohexanecarboxylic acid, TFA salt;
2-{(S)-3-[5-(2,6-dimethyl-benzoyl)-hexahydro-pyrrolo[3,4-c]pyrrol-2-yl]-1-phenyl-propylcarbamoyl}-cyclopentanecarboxylic acid;
(3-{(S)-3-[5-(2,6-dimethyl-benzoyl)-hexahydro-pyrrolo[3,4-c]pyrrol-2-yl]-1-phenyl-propyl}-ureido)-acetic acid;
4-(3-{(S)-3-[5-(2,6-dimethyl-benzoyl)-hexahydro-pyrrolo[3,4-c]pyrrol-2-yl]-1-phenyl-propyl}-ureido)-butyric acid;
(S)-1-methanesulfonyl-pyrrolidine-2-carboxylic acid {(S)-3-[5-(4,6-dimethyl-pyrimidine-5-carbonyl)-hexahydro-pyrrolo[3,4-c]pyrrol-2-yl]-1-phenyl-propyl}-amide;
(1R,2S)-cyclopentane-1,2-dicarboxylic acid 1-dimethylamide 2-({(S)-3-[5-(4,6-dimethyl-pyrimidine-5-carbonyl)-hexahydro-pyrrolo[3,4-c]pyrrol-2-yl]-1-phenyl-propyl}-amide), TFA salt;
3-methoxy-cyclobutanecarboxylic acid [(S)-3-[5-(4,6-dimethyl-pyrimidine-5-carbonyl)-hexahydro-pyrrolo[3,4-c]pyrrol-2-yl]-1-(3-fluoro-phenyl)-propyl]-amide;
3-acetylamino-cyclobutanecarboxylic acid [(S)-3-[5-(4,6-dimethyl-pyrimidine-5-carbonyl)-hexahydro-pyrrolo[3,4-c]pyrrol-2-yl]-1-(3-fluoro-phenyl)-propyl]-amide;
3-(acetyl-methyl-amino)-cyclobutanecarboxylic acid [(S)-3-[5-(4,6-dimethyl-pyrimidine-5-carbonyl)-hexahydro-pyrrolo[3,4-c]pyrrol-2-yl]-1-(3-fluoro-phenyl)-propyl]-amide;
3-methanesulfonylamino-cyclobutanecarboxylic acid [(S)-3-[5-(4,6-dimethyl-pyrimidine-5-carbonyl)-hexahydro-pyrrolo[3,4-c]pyrrol-2-yl]-1-(3-fluoro-phenyl)-propyl]-amide, TFA salt;
3-(methanesulfonyl-methyl-amino)-cyclobutanecarboxylic acid [(S)-3-[5-(4,6-dimethyl-pyrimidine-5-carbonyl)-hexahydro-pyrrolo[3,4-c]pyrrol-2-yl]-1-(3-fluoro-phenyl)-propyl]-amide; and,
hexanoic acid (1R,3R)-3-[(S)-3-[5-(4,6-dimethyl-pyrimidine-5-carbonyl)-hexahydro-pyrrolo[3,4-c]pyrrol-2-yl]-1-(3-fluoro-phenyl)-propylcarbamoyl]-cyclopentyl ester, TFA salt.

13. A method for treating or preventing an human immunodeficiency virus (HIV) infection, or treating AIDS or ARC, in a patient in need thereof which comprises administering to the patient a therapeutically effective amount of a compound of claim 1.

14. A method according to claim 13 further comprising co-administering at least one compound selected from the group consisting of HIV nucleoside reverse transcriptase inhibitors, HIV non-nucleoside reverse transcriptase inhibitors, HIV protease inhibitors and viral fusion inhibitors.

15. A method according to claim 14 wherein the non-nucleoside reverse transcriptase inhibitor is selected from the group consisting of efavirenz, nevirapine and delavirdine, and/or the nucleoside reverse transcriptase inhibitor is selected from the group consisting of zidovudine, didanosin, zalcitabine, stavudine, lamivudine, abacavir, adefovir and dipivoxil, and/or the protease inhibitor is selected from the group consisting of saquinavir, ritonavir, nelfinavir, indinavir, amprenavir and lopinavir and/or the viral fusion inhibitor is T-20.

16. A method of treating a mammal with a disease state that is alleviated by a CCR5 receptor antagonist wherein said disease is solid organ transplant rejection, graft v. host disease, arthritis, rheumatoid arthritis, inflammatory bowel disease, atopic dermatitis, psoriasis, asthma, allergies or multiple sclerosis which comprises administering to the mammal in need thereof a therapeutically effective amount of a compound of claim 1.

17. A method of claim 16 further comprising co-administering at least one other immune modulator.

18. A method of claim 16 where the mammal is a human.

19. A pharmaceutical composition for treating or preventing an human immunodeficiency virus (HIV) infection, or treating AIDS or ARC comprising a compound according to claim 1 admixed with at least one pharmaceutical acceptable carrier, diluent or excipient.

20. A pharmaceutical composition for treating a mammal with a disease state that is alleviated by a CCR5 receptor antagonist wherein said disease is solid organ transplant rejection, graft v. host disease, arthritis, rheumatoid arthritis, inflammatory bowel disease, atopic dermatitis, psoriasis, asthma, allergies or multiple sclerosis comprising a compound according to claim 1 admixed with at least one pharmaceutical acceptable carrier, diluent or excipient.
